(19) United States
(12) Patent Application Publication
(10) Pub. No.: US 2023/0346940 A1
(43) Pub. Date: Nov. 2, 2023

(54) Method And System For Identifying And Validating Shared Candidate Antigens And Shared Antigen-Specific T Lymphocyte Pairs

(71) Applicant: National University of Singapore, Singapore (SG)

(72) Inventors: David Michael EPSTEIN, Singapore (SG); Raymond LEE, Singapore (SG); Shu Cheng WONG, Singapore (SG)

(21) Appl. No.: 17/756,304

(22) PCT Filed: Nov. 20, 2020
(86) PCT No.: PCT/SG2020/050681

§ 371 Date: May 20, 2022

(30) Foreign Application Priority Data
Nov. 22, 2019 (SG) 10201911055U

(51) Int. Cl. A61K 39/00; G16B 20/20; G01N 33/58; G01N 33/574; C07K 14/725; C07K 16/28; C07K 16/40; C07K 16/32

(52) U.S. Cl. CPC A61K 39/464462; G16B 20/20; G01N 33/58; G01N 33/574; C07K 14/7051; C07K 16/2833; C07K 16/40; A61K 39/4613; A61K 39/4632; A61K 39/464414; C07K 16/32; C07K 16/286; A61K 39/464401; A61K 2239/28

(57) ABSTRACT

The present invention relates to a method and system for identifying and validating pairs of candidate antigens and their cognate antigen-specific T lymphocytes that are useful for validating the immunogenic activity of paired antigen and TCR sequences. The method includes, inter alia, steps of determining one or more splice variants that are more highly transcribed in a sample obtained e from cohort of patients compared to a reference sample, determining one or more amino acid sequences that occur in an amino acid translation of said one or more splice variants but not in the corresponding splice variant in the reference sample, and predicting HLA binding of the amino acid sequences in order to identify candidate shared antigen. The present invention also relates to methods of characterising and/or treating a medical condition, including cancer.

### TECHNICAL FIELD

The present invention relates to a method and a system for identifying and validating pairs of candidate antigens and their cognate antigen-specific T lymphocytes that are useful for validating the immunogenic activity of paired antigen and TCR sequences and for characterising and/or treating a medical condition.

### BACKGROUND

Immunotherapy has recently increased greatly in importance. This is particularly the case in relation to treatment or prevention of cancers, though immunotherapies have application in relation to other medical conditions, such as allergies.

Various immunotherapeutic techniques are known, including activation immunotherapies such as dendritic cell-based priming and T-cell adoptive transfer, and autologous immune enhancement therapy using T lymphocytes.

A problem that arises in development of immunotherapies is the identification of functional target antigens and their cognate T cells and/or T cell receptor sequences. One commonly adopted approach in relation to cancer immunotherapies is to search for candidate neoantigens derived from somatic mutations in tumour cells, for example by deep sequencing of tumour DNA or RNA. One goal of this approach is to develop neoantigen-based cancer vaccines which are highly tailored to the mutation profile of individual patients. This highly personalized approach to cancer immunotherapy requires that cancer patients submit their tumour DNA for deep sequencing, whereupon in silico analyses are completed to identify candidate antigenic peptide sequences that can be used to define an individual cancer vaccine for use in cancer treatment. This approach is slow, cumbersome, expensive, and is dependent on a best-guess for the antigenicity of candidate peptides. Importantly, this approach does not afford rapid and simultaneous identification of cognate T cells and TCR sequences. Furthermore, it does not lend itself to rapid and/or simultaneous functional validation of such cognate T cells and TCR sequences useful in the development of cancer therapeutics. Another difficulty with this approach is that although cancer is a disease that is often driven by oncogenic mutation, the mutation pattern defining the genetic profile of individual patients is unique and the oncogenicity of most somatic mutations is unknown, as is the antigenicity of most somatic mutations. A recent study of the antigenicity of cancer associated somatic mutations found that less than 1 in 1,000 candidate antigenic peptides derived from missense mutations lead to a functional neo-antigen, and furthermore this analysis did not result in the identification of a T Cell or TCR pair from which to develop a tailored therapy. Indeed somatic mutations are very rare, when examined at the level of the individual cancer patient, typically being present in less than 0.5% of tumour cells. Accordingly, it can be difficult to identify functional cancer neoantigens that arise from somatic mutations.

There are additional limitations of the current approach whereby potential cancer neoantigens are predicted by exon sequencing of biopsy tissue to identify missense mutations in cancer-associated proteins. Following the analysis of the exon sequencing data, in silico HLA-peptides are then predicted using algorithms designed to prioritize potential antigenic peptide sequences; these predicted amino acid sequences are then utilised (through a variety of peptide-, RNA-, or DNA-based approaches) to develop patient-specific cancer vaccines. There are major limitations to this approach. Because most DNA mutations do not result in tumour-driver events, these ‘private mutations’ are not selected for during tumour evolution, and when viewed at a population level such ‘private mutations’ neither accumulate nor recur and hence do not cluster into discrete patient subgroups. In contrast, there are numerous examples of tumour-driver mutations that exhibit high degrees of recurrence; e.g., BRAF-V600E in melanoma, EGFR kinase domain mutations in lung cancer, HER2 amplifications and mutations in breast cancer or kRas mutations in pancreatic, colorectal, and lung cancers. Of interest is the observation that the aforementioned oncogenic mutations are not particularly immunogenic and have not led to the successful development of targeted immunotherapies. Hence, there is a significant need to develop methods and systems that afford rapid, efficient identification and validation of cancer antigens that are shared across cancer patient populations. Additionally, peptide antigen prediction algorithms may not be suitable for predicting peptide binding to certain HLA alleles, such as those common in Asia. Conversely, peptide neoantigens predicted to bind to Asian-specific HLA alleles may not be suitable as binding partners for HLA alleles common to non-Asian populations. Hence, what is needed is a robust system and methodology that affords evaluation of any candidate antigen-HLA complex so that screening for antigen-T lymphocyte pairs can be completed using any HLA subtype. There is a substantial need for systems and methods that afford rapid, efficient identification and characterization of cognate antigen-directed T cell partners and T cell receptors that bind to and destroy tumours that present shared antigens. Taken together, there remains a need to be able to rapidly identify and validate functional immuno-modulatory pairs of shared cancer antigens and the antigen-specific T lymphocyte.

It has also previously been proposed that neoantigens could arise from dysregulated mRNA splicing events. However, development of cancer immunotherapies via this approach remains challenging. For example, there is difficulty in identifying mRNA splicing events that are tumour-specific, a step that is essential to identify cancer-associated splice variant proteins (SVP) that can be assessed as a source of splicing derived antigens. It is also of critical importance to develop a system which robustly delineates cancer-associated changes in RNA splicing to minimize the risk of off-target toxicity arising from immunotherapies directed to antigens that are present in both disease and normal tissues. There are also significant challenges in detecting protein variants derived from dysregulated mRNA splicing events, because many alternative splicing events are found in transcripts that exist in low abundance. Even when evidence is found that a splice variant is translated into protein, it is not guaranteed that peptides derived from the full-length protein will have immunogenic activity.

Accordingly, it is generally desirable to overcome or ameliorate one or more of the above-mentioned difficulties. The present inventions provide solutions to these problems in the discovery and development of precision immune-therapies.

### SUMMARY

The present invention is predicated on the realization that dysregulated pre-mRNA splicing events are shared among patient subgroups, and that peptides derived from protein splice-forms address limitations of exploiting neo-antigens in immunotherapy. The method as defined herein teaches identification and validation of one or more candidate antigens that is shared by a subgroup of patients having a particular medical condition (such as cancer). This may allow the rapid development of diagnostic tests and treatment options for this subgroup of patients based on the one or more validated shared candidate antigens. The methods as defined herein also teach identification and validation of one or more cognate T lymphocytes and T-cell receptors (TCRs) that bind to and recognize one or more shared antigens derived from dysregulated mRNA splicing: the methods further teach that a shared antigen and its cognate T lymphocyte are also shared by a subgroup of patients having a particular medical condition (such as cancer). This also may allow the rapid development of T cell treatment options for this subgroup of patients based on the one or more validated shared antigens. The methods as defined herein also teach the parallel validation of one or more pairs of antigens and cognate T lymphocytes. These pairs are also shared by a subgroup of patients having a particular medical condition (such as cancer). This also may allow the rapid development of TCR-based treatment options for this subgroup of patients based on the one or more validated immunotherapeutic pairs.

It has not been previously demonstrated that aberrant mRNA splicing events that lead to cancer-associated changes in protein sequence, via changes in coding sequences, could also lead to the presentation of antigenic peptides in cancer patients. Nor has it been previously determined whether tumour-associated splicing changes in cancer patients would lead to the development of shared cancer antigens, or that such candidate shared antigenic peptides are displayed on tumour cells, and/or that such surface-displayed HLA-peptide antigens could bind to and activate cognate T lymphocytes that functionally kill tumour cells that harbour the shared mRNA splicing event.

Disclosed herein is a method of identifying one or more shared candidate antigens for characterising and/or treating a medical condition, the shared candidate antigens being common to a subset of patients having the medical condition, the method including:
(i) obtaining transcriptomic data for test samples from a first cohort of patients having the medical condition;
(ii) obtaining reference transcriptomic data for a set of reference samples;
(iii) determining, by a comparison of the transcriptomic data to the reference transcriptomic data, one or more splice variants that are more highly transcribed in each sample of a subset of the test samples as compared to the reference samples;
(iv) determining, for each said shared splice variant, one or more amino acid sequences that occur in an amino acid translation of the shared splice variant, but not in amino acid translations of corresponding splice variants of the same gene that are transcribed in the reference samples; and
(v) predicting HLA binding of the one or more shared amino acid sequences, or part thereof, to identify the one or more shared amino acid sequences as one or more shared candidate antigens.

Disclosed herein is a method of identifying a shared antigen-T lymphocyte pair, the method comprising:
a) identifying a shared candidate antigen according to a method as defined herein;
providing one or more respective labelled biomolecules comprising a label and a peptide comprising the shared candidate antigen;
b) contacting the one or more labelled biomolecules with one or more samples containing peripheral blood from patients having the medical condition; and
c) identifying, from the one or more samples, T lymphocytes that are bound to said labelled biomolecules, so as to identify a shared antigen-T lymphocyte pair.

Disclosed herein is a method for identifying T lymphocytes that bind specifically to one or more shared candidate antigens identified according to a method as defined herein, comprising:
a) providing one or more respective labelled biomolecules comprising a label and a respective candidate antigen;
b) contacting the one or more labelled biomolecules with one or more samples containing peripheral blood from respective patients having the medical condition; and
c) identifying, from the one or more samples, T lymphocytes that are bound to said labelled biomolecules.

Disclosed herein is a method of characterising a medical condition in a subject, the method comprising determining the level of one or more shared antigens identified according to a method as defined herein, wherein an increased level of the one or more shared antigens as compared to a reference characterises the medical condition as one that is associated with the expression of the one or more shared antigens.

A medical condition that is associated with the expression of the one or more shared antigens as defined herein also indicates that the medical condition is likely to be responsive to treatment with a suitable immunotherapy.

Disclosed herein is a method of treating a medical condition in a subject, the method comprising (a) determining the level of one or more shared antigens identified according to a method as defined herein, wherein an increased level of the one or more shared antigens as compared to a reference characterises the medical condition in the subject as one that is associated with the expression of the one or more shared antigens, and (b) treating the subject found to have a medical condition associated with the expression of the one or more shared antigens.

Disclosed herein is a method of characterising a medical condition in a subject, the method comprising determining the level of T lymphocytes that binds specifically to one or more shared antigens identified according to a method as defined herein, wherein an increased level of the T lymphocytes as compared to a reference characterises the medical condition in the subject as one that is associated with the expression of the one or more shared antigens.

Disclosed herein is a method of treating a medical condition in a subject, the method comprising a) determining the level of T lymphocytes that bind specifically to one or more shared antigens identified according to a method as defined herein, wherein an increased level of the T lymphocytes as compared to a reference characterises the medical condition in the subject as one that is associated with the expression of the one or more shared antigens; and b) treating the subject found to have a medical condition associated with the expression of the one or more shared antigens.

Disclosed herein is a method of treating a medical condition in a subject, the method comprising:
(a) determining the level of T lymphocytes that binds specifically to one or more shared antigens as defined herein, wherein an increased level of the T lymphocytes as compared to a reference characterises the medical condition as one that is associated with the expression of the one or more shared antigens;
(b) isolating and expanding the population of T lymphocytes ex vivo; and
(c) administering the expanded population of T lymphocytes to the subject to treat the medical condition found to be associated with the expression of the one or more shared antigens.

Disclosed herein is a method of treating a medical condition in a subject, the method comprising:
(a) isolating a population of T lymphocytes that binds specifically to one or more shared antigens identified according to a method as defined herein in a subject suffering from the medical condition, and expanding the population of T lymphocytes ex vivo; and
(b) administering the expanded population of T lymphocytes to the subject to treat the medical condition in the subject.

Disclosed herein is an immunomodulatory composition comprising one or more shared antigens identified according to a method as defined herein and a pharmaceutically acceptable carrier.

Disclosed herein is a method of stimulating an immune response in a subject, the method comprising administering an effective amount of an immunomodulatory composition according a method as defined herein to the subject under conditions and for a sufficient time to stimulate the immune response in the subject.

Disclosed herein is a shared antigen-T lymphocyte pair identified according to a method as defined herein, wherein the shared antigen is a MARK3, NBPF9, PARD3, ZC3HAV1, YAF2, CAMKK1, LRR1, ZNG670, GRINA or MZF1 splice variant, the HLA subtype is HLA-A11 or HLA-A24, and the T lymphocyte binds to the shared antigen.

In one embodiment, there is provided a shared antigen-T lymphocyte pair identified according to a method as defined herein, wherein the shared antigen is a MARK3 splice variant, the HLA subtype is HLA-A11, and the T lymphocyte binds to the shared MARK3 antigen.

Disclosed herein is a labelled biomolecule comprising a HLA molecule bound to a shared antigen for use in detecting the presence or determining the level of T lymphocytes that binds specifically to the shared antigen.

Disclosed herein is an antibody that binds specifically to a shared antigen identified according to a method as defined herein, wherein the shared antigen is bound to a HLA molecule.

Disclosed herein is a T-cell receptor (TCR) that binds to a shared antigen identified according to a method as defined herein, wherein the shared antigen is bound to HLA molecule.

Disclosed herein is an engineered immune cell comprising a nucleic acid encoding a T-cell receptor as defined herein, wherein the engineered immune cell is capable of specifically binding to a shared antigen or fragment thereof, wherein the shared antigen or fragment thereof is bound to a HLA molecule.

Disclosed herein is a method of treating a medical condition in a subject, the method comprising administering a TCR as defined herein or an engineered immune cell as defined herein to the subject for a sufficient time and under conditions to treat the medical condition in the subject.

Disclosed herein is a method of producing an antibody, the method comprising:
(a) immunizing an animal with a shared antigen identified according to a method as defined herein;
(b) identifying and/or isolating a B cell from the animal, which binds specifically to the shared antigen; and
(c) producing the antigen-binding molecule expressed by that B cell.

Disclosed herein is a pharmaceutical composition comprising an antibody, a solubilised TCR or an engineered immune cell as defined herein, and a pharmaceutically acceptable carrier.

Disclosed herein is a method of treating a medical condition in a subject, the method comprising administering a pharmaceutical composition as defined herein to the subject for a sufficient time and under conditions to treat the medical condition in the subject.

Disclosed herein is method of identifying a shared antigen-T lymphocyte pair, the method comprising:
(i) obtaining transcriptomic data for test samples from a first cohort of patients having the medical condition, wherein the cohort comprises a plurality of patients;
(ii) obtaining reference transcriptomic data for a set of reference samples;
(iii) determining, by a comparison of the transcriptomic data to the reference transcriptomic data, one or more splice variants that are more highly transcribed in each sample of a subset of the test samples as compared to the reference samples,
(iv) determining, for each said shared splice variant, one or more amino acid sequences that occur in an amino acid translation of the splice variant, but not in amino acid translations of corresponding splice variants of the same gene that are transcribed in the reference samples;
(v) predicting HLA binding of the one or more shared amino acid sequences, or part thereof, to identify the one or more amino acid sequences as one or more shared candidate antigens;
(vi) providing one or more labelled biomolecules comprising a label and a peptide comprising a shared candidate antigen;
(vii) contacting the one or more labelled biomolecules with one or more samples containing peripheral blood from patients having the medical condition; and
(viii) identifying, from the one or more samples, T lymphocytes that are bound to said labelled biomolecules, so as to identify a shared antigen-T lymphocyte pair.

### BRIEF DESCRIPTION OF DRAWINGS

Embodiments of the present invention are hereafter described, by way of non-limiting examples only, with reference to the accompanying drawings in which:

FIG. 1(a) is a flow diagram of a method for identifying candidate antigens for characterising and/or treating a medical condition.

FIG. 1(b) is a flow diagram of a method for identifying antigen-specific T lymphocytes.

FIG. 2 is a schematic workflow of a method for identifying shared candidate antigens for characterising and/or treating a medical condition.

FIG. 3 shows examples of ridge-plots for the distribution of PSI values in normal and tumour samples. Ridge-plots for ten splicing events are shown. In some of these examples, the “outliers” are shown in dotted line boxes. These “outliers” are tumour samples that have PSI values that are different from the remainder of the tumour samples.

FIG. 4 shows an example of a sashimi plot from two patients with a set of tumour and normal samples. The sashimi plots show the density of sequencing reads that map to the junctions of the exons as well as the exons themselves. Based on the sequencing read density, it is possible to infer the splice variant isoforms being expressed in the sample. The numbers shown refer to the number of reads that span the splice junction. In this example, based on the sashimi plots, the normal samples (numbers ending in ‘N’) show increased expression of the skipped splice variant isoform, whereas the tumour samples (numbers ending in ‘T’) show increased expression of the splice variant isoform with inclusion of the middle exon.

FIG. 5 illustrates types of splicing events that are observed and their corresponding potential candidate antigenic region. There are five types of splicing events that are observed; namely: SIE—skipped/inclusion events; MXE—alternate usage of exons; IRE—intron retention events; A5E—alternate 5′ splicing events; and A3E—alternate 3′ splicing events. Each of these splicing events can give rise to two splice isoforms and one of them would be more likely to be cancer-associated. Addition of sequences (through differential use of exons or introns or parts thereof) might lead to changes in translation frame (right side of figure shown by ‘, for example a’). This might have an impact on the antigenic region (shown by light grey lines in this Figure) being used for subsequent prediction of HLA binding peptides.

FIG. 6 (a) is a schematic depiction of a method to determine whether a splicing alteration results in change in protein sequence, and the amino acid region that differs between the splice isoforms, for identifying potential HLA binding peptides. For each splicing event, the translation frame for exon 1 is determined and is discarded if it is non-coding or it is coding but contains a stop codon. If it is coding, exon 1 is translated based on the position of the start codon or the translation frame obtained for this exon according to a database (for example, Ensembl). This is done for both isoforms (isoform 1 and 2). The antigenic region is determined (i) by whether a change in pattern of splicing causes a change in translation frame and (ii) by comparing the protein sequence of the two isoforms, shown in greater detail in FIG. 6(b). b) is an example illustrating the method for determining the amino acid sequence of a potential candidate antigenic region of a splicing event. Each splicing event give rises to two splice isoforms (Tumour-associated [TA] and non-tumour-associated [N]) and the potential candidate antigenic region is shown (underlined text, comprised of two components: N-term and C-term). The length of the flanking region (a-h) plus the amino acid at the junction of the splice site (J) equals the length of HLA binding peptide (9 in this example). The N-term and C-term of the splice site of each splice isoform are compared separately to determine how many amino acids long the N-term and C-term flanking regions are. In the event that the splicing event causes a change in frame, then the C-term flanking region consists of all the amino acid sequence of the last exon. The amino acid sequence of the underlined region of the two splice isoforms are compared iteratively (starting with the junctional amino acids J_{T} and J_{N}, followed by A_{T1} and A_{N1}, etc). If they are the same, then an amino acid from the flanking region of the tumour-associated isoform (A_{TX}, where X refers to the outermost amino acid, starting from a to h in this example) is removed; otherwise the process is stopped. The antigenic region consists of joining the results from the comparison of the N-term and C-term regions of the splicing event. Additionally, if the splicing event leads to the inclusion of additional sequence from the inclusion of introns or exons or parts thereof, the potential candidate antigenic region would contain amino acid sequence from the translation of these sequences. Assume we are looking for: (i) potential HLA binding peptides that are 9 amino acids long; (ii) the exon skipped isoform has been shown to be tumour-associated; (iii) the amino acid at the junction of the tumour-associated and non-tumour-associated splice isoform is the same (J_{T}=J_{N}); and (iv) splicing event does not cause a change in translation frame. The potential candidate antigenic region from this set of assumptions is shown at the bottom (Example final output) for a tumour-associated exon-skipping event. In this example the potential candidate antigenic region comprises removal of one flanking amino acid from both ends of the initial 8 amino acid long flanking region.

FIG. 7 is a schematic diagram showing preparation of HLA tetramer-splice variant candidate antigen complexes for characterization of T lymphocytes derived from cancer patients.

FIG. 8 is a block diagram of an example system for identifying candidate antigens for characterising and/or treating a medical condition; and

FIG. 9 is a block diagram of an example architecture of an antigen prediction apparatus of the system of FIG. 8.

FIG. 10 is a schematic representation showing a workflow for deriving antigens from splicing in gastric cancer. Briefly, RNA-Seq data from gastric cancer (GC) patients was analyzed for splicing alterations using MISO. Selection criteria (Top 0.5% splicing events, at least 20% change in splicing (ΔPSI), Bayes factor >20, and occurrence in at least 3 patients) were applied to the data to generate a list of splicing events. We then looked for splice events that led to a change in the protein sequences. These protein regions (291 protein regions) resulting from altered splicing were then used to predict peptides that were 8-11 amino acid long peptides that could bind to HLA A11 (total of 39,876 peptides). NetMHCpan3 was used for predicting HLA binding, and it returned 153 peptides that had high affinity for HLA A11 (Rank <=0.5%). This list was further reduced to 77 peptides by removing peptides that were similar.

FIG. 11 is a schematic and graphical representation showing a summary of GC tumour-associated splicing alterations identified in a 19-patient cohort: (a) Summary of the types of splicing alterations seen in gastric cancer. Specifically, it was found that there are 5 different types of splicing events that are deregulated namely: Exon skip/inclusion; Alternate usage of Exons, intron retention and alternate 5′ and alternate 3′ splices sites. The majority of these events are skip/inclusion of exons; (b) Distribution of GC-ASEs: prevalence of MARK3 splicing-derived peptide antigen. Histogram of the occurrence and PSI for the splice events identified in GC.

FIG. 12 is a graphical representation showing: (a) Identification of MARK3 peptide from GC TA-ASE dataset CyTOF screen in GC patients. Histogram showing that patient SC020 has CTLs that react with a peptide derived from aberrant splicing of MARK3. Lower left panel shows the cluster of cells that are stained by the MARK3 splice variant peptide (SVP) in patient SC020. The panels on the right show the phenotype of the CTLs that recognize MARK SVP, using activation, senescence markers and exhaustion markers; and (b) Confirmation of MARK3 peptide using fluorescently labelled A11 MHC tetramers.

FIG. 13 is a graphical representation showing: Positive control peptide in GC TA-ASE peptide screen. Positive control used in CyTOF screen. Peptide shown is derived from Epstein Barr virus (EBV). These EBV antigens are commonly seen in the population. The histogram shows the frequency of these CTLs in the same patient cohort used for screening splicing-derived antigens in GC patients.

FIG. 14 is a graphical, schematic and photographic representation showing: (a) MARK3 GC tumour-associated splicing event median ΔPSI=−0.335, occurrence=4/19 GC patients. Sashimi plots of aberrant splicing of MARK3 in the original GC RNA-Seq. These plots show the counts of sequencing reads that map to exons as well as reads that map to the junctions of the exons and also the PSI value of each sample. Sashimi plots of normal and tumour samples are shown: normal samples have numbers ending in ‘N’, and tumour samples have numbers ending in ‘T’. In these four pairs samples, there is increased inclusion of the alternate exon in tumour samples. Altered splicing of MARK3 is seen in four out of nineteen patients and shows an inclusion of ˜35% of the alternatively spliced exon; (b) Transcripts obtained from Ensembl GRCh37.p13 showing various MARK3 splice isoforms corresponding to isoforms 1 to 4 are shown here; (c) RT-PCR validation of MARK3 aberrant splicing in GC cell lines. Top panels show a cartoon of two alternative exons in MARK3 (Exon 24 in indicated using vertical stripes and Exon 25 indicated using diagonal stripes). The exon with vertical stripes encodes the peptide (shown beneath) that was detected in the CyTOF screen. Alternative splicing of these two exons leads to the formation of four isoforms (1-4), isoform 1 and 3 both contain the peptide detected in the CyTOF screen. Lower panels are RT-PCR showing increased expression of MARK3 isoform 1/3 in HFE145, SNU1, HS738T, HS746T and HGC27 compared to other cell lines (asterix); (d) is a table showing the quantification of the MARK3 splice isoforms 1 to 4 in GC cell lines, percentage of each isoform out of total is shown. The percentage of MARK3 isoforms 1 and 3 out of all MARK3 isoforms is shown, GC cell lines that have increased expression of isoforms 1 and 3 are underlined. Quantification of the MARK3 isoforms was done by densitometry of the intensity of the DNA bands after running the PCR products on a TBE-PAGE gel; and (e) is a photographical representation of the validation of MARK3 aberrant splicing in gastric FFPE sample. RT-PCR validation of MARK3 splicing in FFPE samples from gastric cancer patients (1-20) and bariatric gastric samples (21-26). Numbers indicated below each lane indicate the percentage of isoform 1 and 3 out of the total expression of all MARK3 isoforms. Increased expression of MARK splice isoforms that contain the exon encoding the identified MARK3 splice variant antigen is observed in 7 out of 20 GC patients (underlined samples).

FIG. 15 is a photographic representation showing: (a) the results of an ELISPOT assay for IFN-γ in PBMCs from healthy donors with or without stimulation with MARK3 peptide. CTLs only secrete IFN-γ when they recognize their cognate antigen. From this figure, IFN-γ secreting CTLs are only observed when PBMCs were stimulated with MARK3 peptide; and (b) results of a cell killing assay by MARK3 CTLs. MARK3 specific CTLs can mediate killing of HGC-27 cell line that express MARK3 splice variant antigen and HLA-A11 in a dose-dependent manner.

FIG. 16 is a graphical representation of FACS data for isolation of MARK3 specific CD8+T lymphocytes and single cell sorting of these cells. Purified CD8+ T cells from healthy donor stimulated with antigen-presenting cells loaded with MARK3 peptide were stained with anti-CD3, anti-CD8 antibodies, HLA-A*11 MARK3 pentamer and DAPI before cell sorting. Cells were gated using the forward (FSC-A) and side (SSC-A) scatter area parameter followed by gating for single cells using FSC-A vs height (FSC-H). MARK3 specific CD8+T lymphocytes were identified by gating for DAPI negative live cells, expression of CD8 and CD3, and binding to HLA-A11 MARK3 pentamer. These MARK3 specific CD8+T lymphocytes were then sorted into single cells into a PCR plate for subsequent TCR identification.

FIG. 17 is a schematic and graphical representation of alternative splicing of MARK3 Exon 24 (Exon 17 in TCGA SpliceSeq) in Head and Neck Squamous Cell Carcinoma (HNSC), Kidney Renal Clear Cell Carcinoma (KIRC) and Kidney Renal Papillary Cell Carcinoma (KIRP) in TCGA SpliceSeq database. Normal samples are shown as striped boxes whereas tumour samples are shown as open boxes. From this Figure, tumour samples show an increased inclusion of Exon 24.

FIG. 18 is a photographic representation showing the alternative splice isoform of MARK3 isoforms containing MARK3 SVA that was identified in the CyTOF screen (Example 3): (a) RT-PCR of MARK3 aberrant splicing in head and neck squamous cell carcinoma (HNSC)-derived cell lines is shown here. MARK3 isoforms, corresponding to isoforms 1 to 4 shown in FIG. 14c are indicated in this Figure. HNSC cell lines which show increased expression of MARK splice isoform 1 and 3 (these isoforms contain the MARK3 SVA peptide identified in the CyTOF screen, Example 3) are indicated with an asterisk; (b) table showing the quantification of the MARK3 splice isoforms 1 to 4, HNSC cell lines which have increased expression of isoforms 1 and 3 are underlined. Quantification of the MARK3 isoforms was done by densitometry of the intensity of the DNA bands after running the PCR products on a TBE-PAGE gel.

FIG. 19 is a schematic and graphical representation showing a summary of the identification and validation of shared candidate antigens and their cognate T cells in colorectal cancer: (a) is a schematic representation showing a workflow for deriving antigens from splicing in colorectal cancer. Briefly, RNA-Seq data from 37 colorectal cancer (CRC) patients was analysed for splicing alterations using rMATS. Selection criteria (at least 20% change in splicing (ΔPSI), occurrence in at least 6 patients and splice junction counts greater than 10) were applied to the data to generate a list of splicing events. Splice events that led to a change in the protein sequences were identified and these protein regions (352 candidate antigenic regions) resulting from altered splicing were then used to predict peptides that were 8-11 amino acid long peptides that could bind to HLA A11 (total of 62,970 peptides). NetMHCpan3 and NetMHCpan4 was used for predicting HLA binding, and it returned 425 peptides that had high affinity for HLA A11 (Rank <=0.5% for either algorithm). This list was further reduced to 102 peptides by removing peptides that were similar. An immunological screen to identify antigen specific T-cells was performed in PBMCs from 8 CRC patients (Patient immunological response). This was performed using peptide/HLA tetramers and CyTOF and lead to the identification of antigen specific T-cells against 27 SVPs. The expression of 9 SVPs was confirmed by RT-PCR in cancer cell lines to be differentially spliced. Antigen specific T-cells could be generated in healthy donor PBMC against 3 of these SVP showing that these targets are immunologic. (b) Summary of the types of splicing alterations observed in 37 CRC patients. Specifically, it was found that there are 4 different types of splicing events that are deregulated namely: Exon skip/inclusion (SIE); intron retention (IRE) and alternate 5′ (A5E) and alternate 3′ splices sites (A3E). Most of these events are skip/inclusion of exons. The number of splice events that produce a change in the coding sequence is shown; (c) Distribution of CRC-ASEs and the type of splice event is indicated. Histogram of the occurrence and ΔPSI for the splice events identified in CRC that produced HLA-A11 peptides are shown.

FIG. 20 provides two tables showing the SVP targets that were identified in a CRC HLA-A11 tetramer/CyTOF screen: (a) summary of the HLA-A11 binding peptides that were detected in 8 CRC patients. The frequency of CD8 positive T lymphocytes in these patients that bind to these targets is indicated in the first table. The occurrence, ΔPSI and type of splice events that gave rise to these SVPs are also shown in the first table; (b) summary of the sequence coordinates as well as tumour-associated isoforms that gave rise to the SVPs. These coordinates are based on the human GRCh37/hg19 assembly.

FIG. 21 is a graphical representation of tumour-associated splicing identified in colorectal cancer: (a) Splicing alterations identified in CRC that are tumour-associated and cause changes in protein coding sequence. Each column represents a sample from a CRC patient and each row represents a single splice event. The PSI value for each sample is shown and there is a clear distinction in the PSI values for tumour vs normal samples.

This distinction in PSI values between tumour vs normal samples demonstrates that these tumour-associated splice variants can be exploited for treatment of CRC patients; (b) Histogram showing number of shared tumour-associated splice variants that are present in individual CRC patients. On average, the majority of patients show approximately 90 shared tumour-associated splice variants, and this is not restricted to any molecular subtypes nor to the microsatellite status in CRC patients. This is unlike neoantigens derived from somatic point mutations, which are found mainly in MSI/CMS 1 CRC patients. CMS=consensus molecular subtype; MSI=microsatellite instable.

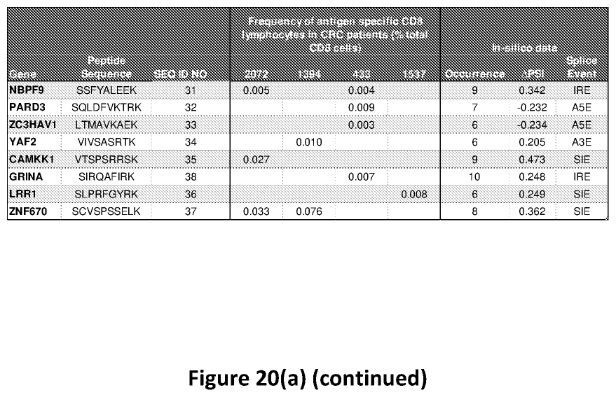

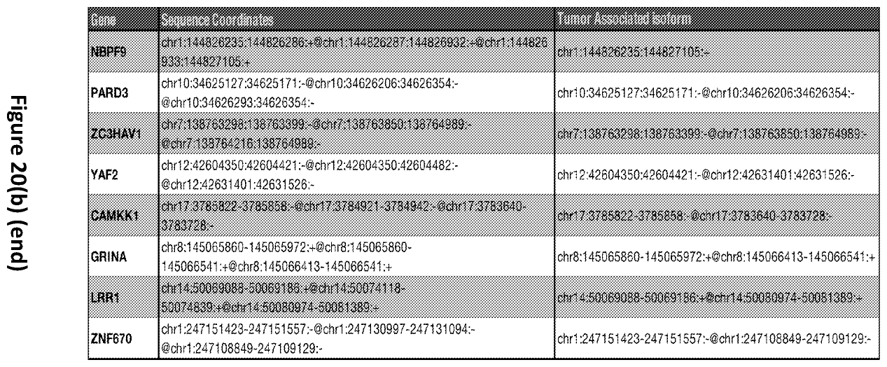

FIG. 22 are schematics, graphical representations and photographic representations showing the alternative splicing of CAMKK1: (a) dot plot showing the PSI value of CAMKK1 in normal (Norm) and tumour (Tum) samples from CRC patients; (b) Sashimi plots showing CAMKK1 splice isoforms that are found in tumour as well as normal samples from CRC patients. Tumour samples show increased exon skipping compared to normal samples. The sashimi plots show the sequencing read density of patients' samples with sufficient junctional counts for a group of normal samples, and a group of tumour samples that are outliers. (c) CAMKK1 Transcripts present in human GRCh37/hg19 assembly. The exon that is alternatively spliced is indicated with a box. Alternative splicing of this exon has not been observed before. The region which is detected by RT-PRC is shown below and it contains an additional alternatively spliced exon (grey box) besides the exon that was identified in this study. These two alternative exons cause the formation of 4 different splice isoforms: the 277 bp and 163 bp splice isoform (both indicated by TA) both contain the HLA-A11 binding peptide that was identified in this study. The 163 bp splice isoform (indicated with TA and asterisk) is the splice isoform that corresponds to the splice isoform that was detected in this study and is shown in the sashimi plot in FIG. 22(b); (d) RT-PCR of CAMKK1 aberrant splicing in CRC cell lines as well as patient-derived biopsy material. Samples that show increased expression of the CAMKK1 tumour-associated splice variants are indicated with an asterisk. The bands that correspond to the tumour-associated splice variants are indicated by TA. The smaller PCR band (163 bp) corresponds to the tumour-associated splice variant that was identified by rMATS; (e) Normal associated DNA and normal protein sequence of CAMKK1 are shown (SEQ ID NOs: 56 and 57). Tumour-associated DNA sequence and tumour-associated protein sequence of CAMKK1 are shown (SEQ ID NOs: 58 and 59). The DNA and amino acid sequences for splice isoforms shown in FIG. 22(b) are shown (labelled as Tumour and Normal). Exon skipping (indicated by Alt Exon) of CAMKK1 that was identified causes a change in protein sequence. The exon that is alternatively spliced contains 22 nucleotides and skipping of this exon leads to changes in the protein translation frame of the transcript. This leads to novel protein sequences and an early termination of the protein. The HLA-A11 binding peptide that was identified in FIG. 20 is underlined; (f) dot plot showing the PSI value of CAMKK1 in normal and tumour samples from HNSC patients; (g) Sashimi plots showing CAMKK1 splice isoforms that are found in tumour as well as normal samples from HNSC patients. Tumour samples show increased exon skipping compared to normal samples. The sashimi plots show the sequencing read density of patients' samples with sufficient junctional counts for a group of normal samples, and a group of tumour samples that are outliers; (h) RT-PCR of CAMKK1 aberrant splicing in HNSC cell lines. Samples that show increased expression of the CAMKK1 tumour-associated splice variant are indicated with an asterisk. The PCR band corresponding to the tumour-associated splice variant is indicated by “TA”.

FIG. 23 are schematic, graphical representation and photographic representation showing the alternative splicing of LRR1: (a) dot plot showing the PSI value of LRR1 in normal and tumour samples from CRC patients. (b) Sashimi plots showing LRR1 splice isoforms that are found in tumour as well as normal samples from CRC patients. Tumour samples show increased exon skipping compared to normal samples. The sashimi plots show the sequencing read density of patients' samples with sufficient junctional counts for a group of normal samples, and a group of tumour samples that are outliers. (c) RT-PCR of LRR1 aberrant splicing in CRC cell lines as well as patient derived biopsy material. Samples that show increased expression of the LRR1 tumour-associated splice variant are indicated with an asterisk. The PCR band corresponding to the tumour-associated splice variant is indicated by “TA”. (d) Normal associated DNA and normal protein sequence of LRR1 are shown (SEQ ID NOs: 60 and 61). Tumour-associated DNA sequence and tumour-associated protein sequence of LRR1 are shown (SEQ ID NOs: 62 and 63). DNA and amino acid sequence for the candidate antigenic region found in the LRR1 tumour-associated splice variant is shown along with the splice variant that includes the alternatively-spliced exon (Alt Exon indicated in diagram). Only partial sequence of the alternatively-spliced exon is shown (indicated by periods) and the skipping of this exon causes a change in the reading frame for the downstream exon. The candidate antigenic region for LRR1 consists of a different C-terminus which is 62 amino acid long and produces two peptides (SLPRFGYRK and SYHSIPSLPRF, SEQ ID NO: 36 and SEQ ID NO: 51 respectively) that can bind to two different HLA alleles (HLA-A11 and HLA-24, respectively). Antigen specific CD8+ T cells specific for these two peptides were detected in CRC patients. (e) dot plot showing the PSI value of LRR1 in normal and tumour samples from HNSC patients. (f) Sashimi plots showing LRR1 splice isoforms that are found in tumour as well as normal samples from HNSC patients. Tumour samples show increased exon skipping compared to normal samples. The sashimi plots show the sequencing read density of patients' samples with sufficient junctional counts for a group of normal samples, and a group of tumour samples that are outliers. (g) RT-PCR of LRR1 aberrant splicing in HNSC cell lines. Samples that show increased expression of the LRR1 tumour-associated splice variant are indicated with an asterisk. The PCR band corresponding to the tumour-associated splice variant is indicated by “TA”.

FIG. 24 are schematics, graphical representations and photographic representations showing the alternative splicing of ZNF670. (a) Dot plot showing the PSI value of ZNF670 in normal and tumour samples from CRC patients. (b) Sashimi plots showing ZNF670 splice isoforms that are found in tumour as well as normal samples from CRC patients. Tumour samples show increased exon skipping compared to normal samples. The sashimi plots show the sequencing read density of patients' samples with sufficient junctional counts for a group of normal samples, and a group of tumour samples that are outliers. (c) RT-PCR of ZNF670 aberrant splicing in CRC cell lines as well as patient-derived biopsy material. Samples that show increased expression of the ZNF670 tumour-associated splice variant are indicated with an asterisk. (d) ZNF670 Transcripts present in human GRCh37/hg19 assembly. The exon that is alternatively spliced is indicated with a box. Alternative splicing of this exon has not been observed before. (e) Normal associated DNA and normal protein sequence of ZNF670 are shown (SEQ ID NOs: 64 and 65). Tumour-associated DNA sequence and tumour-associated protein sequence of ZNF670 are shown (SEQ ID NOs: 66 and 67). Exon skipping of ZNF670 that was identified causes a change in protein sequence. The exon that is alternatively spliced contains 98 nucleotides and skipping of this exon leads to changes in the protein translation frame of the transcript. This aberrant splicing event leads to novel protein sequences comprising changes in the C-terminus of the protein. The HLA-A11 binding peptide that was identified in FIG. 20 is underlined.

FIG. 25 are schematics, graphical representations and photographic representations showing the alternative splicing of GRINA: (a) dot plot showing the PSI value of GRINA in normal and tumour samples from CRC patients. (b) Sashimi plots showing GRINA splice isoforms that are found in tumour as well as normal samples from CRC patients. Tumour samples show increased skipping compared to normal samples. The sashimi plots show the sequencing read density of patients' samples with sufficient junctional counts for a group of normal samples, and a group of tumour samples that are outliers. (c) RT-PCR of GRINA aberrant splicing in CRC cell lines as well as patient-derived biopsy material. Samples which show increased expression of the GRINA tumour-associated splice variant are indicated with an asterisk. The PCR band corresponding to the tumour-associated splice variant is indicated by “TA”. (d) dot plot showing the PSI value of GRINA in normal and tumour samples from HNSC patients. (e) Sashimi plots showing GRINA splice isoforms that are found in tumour as well as normal samples from HNSC patients. Tumour samples show increased skipping compared to normal samples. The sashimi plots show the sequencing read density of patients' samples with sufficient junctional counts for a group of normal samples, and a group of tumour samples that are outliers. (f) RT-PCR of GRINA aberrant splicing in HNSC cell lines. Samples that show increased expression of the GRINA tumour-associated splice variant are indicated with an asterisk. The PCR band corresponding to the tumour-associated splice variant is indicated by “TA”.

FIG. 26 is a graphical representation of FACS data for antigen-specific CD8+ T-cells generated for identified CRC HLA-A11 SVPs. PBMCs from healthy donors (HSA27 and HSA38) were used to generate moDC, which were subsequently used for co-culture with CD8 positive T cells from the same donor. SVPs (LRR1, GRINA and ZNF670) that bind to HLA-A11 and identified in the CRC HLA-A11/CyTOF screen (as shown in FIG. 20) were added during moDC/CD8+ T-cell co-culture to stimulate the expansion of antigen-specific T-cells against these SVPs. Antigen-specific CD8+ T cells were detected using SVP tetramers that were labelled with APC and PE: two fluorescent dyes were used to increase the specificity of detecting these antigen-specific T-cells. Antigen-specific T-cells were observed only after stimulation of CD8+ T-cells with moDC (bottom row). By contrast these antigen-specific T-cells were absent in CD8 positive T-cells in unstimulated PBMCs.

FIG. 27 provides two tables showing the SVA targets that were identified in a CRC HLA-A24 tetramer/CyTOF screen: (a) summary of the HLA-A24 binding peptides that were detected in 10 CRC patients. The frequency of antigen-specific CD8 positive T lymphocytes present in the patient having ID ‘1466’ is shown in the table. The occurrence, ΔPSI and type of splice events that gave rise to these SVAs are also shown; (b) summary of the sequence coordinates as well as tumour-associated isoforms that gave rise to the SVAs. These coordinates are based on the human GRCh37/hg19 assembly.

FIG. 28 are schematics, graphical representations and photographic representations showing the alternative splicing of MZF1: (a) Dot plot showing the PSI value of MZF1 in normal and tumour samples from CRC patients; (b) Sashimi plots showing MZF1 splice isoforms that are found in tumour as well as normal samples from CRC patients. Tumour samples show increased intron retention compared to normal samples. The sashimi plots show the sequencing read density of patients' samples with sufficient junctional counts for a group of normal samples, and a group of tumour samples that are outliers. (c) RT-PCR of MZF1 aberrant splicing in CRC cell lines as well as patient-derived biopsy material. Samples that showed increased expression of the MZF1 tumour-associated splice variant are indicated with an asterisk. The PCR band corresponding to the tumour-associated splice variant is indicated by “TA”.

FIG. 29 is a graphical and schematic representation showing a summary of HNSC tumour-associated splicing alterations identified in a cohort of 31 patients: (a) Summary of the types of splicing alterations seen in HNSC. Specifically, it was found that there are four (4) different types of splicing events that are deregulated; namely: Exon skip/inclusion (SIE); intron retention (IRE); alternate 5′ (A5E) and alternate 3′ splices sites (A3E). The majority of these events are skip/inclusion of exons. The number of splice events that produce a change in the coding sequence is shown; (b) The occurrence and change in PSI for the splice events identified in HNSC are shown in the histogram. Distribution of HNSC-ASEs and the type of splice event are indicated; (c) Number of splice events that produced 8-11 amino acid long peptides that could bind to HLA alleles like HLA-A11, HLA-A02 and HLA-A24 (present in 25-50% of the population) are shown.

FIG. 30 is a table showing the SVP targets that were identified in the CRC HLA-A11/HLA-A24 tetramer/CyTOF screen (as described in Example 13 and Example 21) that are also found in the tumour-associated splice variants present in HNSC. The occurrence, ΔPSI and type of splice events that gave rise to these SVAs in HNSC patients are shown in the table.

### DETAILED DESCRIPTION

Embodiments of the present invention generally relate to identification of HLA-binding peptides, arising from alternative splicing events, which are capable of forming peptide-HLA (pHLA) complexes for presentation to T lymphocytes. The peptides identified by embodiments of the method may be referred to as splice-variant antigens. Embodiments also relate to identification of T lymphocytes that recognise such pHLA complexes, also referred to herein as antigen-specific T lymphocytes. Advantageously, the presently disclosed embodiments identify splice-variant antigens that are shared across more than one patient suffering from a medical condition, rather than seeking to identify patient-specific antigens.

Screening for antigen-specific T lymphocytes using methods such as mass cytometry becomes greatly simplified, since it is possible to prepare a single library of splice variant antigens derived from cancer patients, and look for antigens that are shared among a patient sub-group. Additionally, once it is known that an antigen is shared across patients, it becomes possible to develop screening tests to identify patients falling within this sub-group, and to develop a suitable immunotherapy using the antigen-specific T lymphocytes. Such immunotherapies can then be administered to the patient sub-group for whom it has been determined that the therapy may be effective in the treatment of a particular cancer.

Method for Identifying Candidate Antigens

Disclosed herein is a method of identifying one or more shared candidate antigens for characterising and/or treating a medical condition, the method including:
(i) obtaining transcriptomic data for test samples from a first cohort of patients having the medical condition;
(ii) obtaining reference transcriptomic data for a set of reference samples;
(iii) determining, by a comparison of the transcriptomic data to the reference transcriptomic data, one or more splice variants that are more highly transcribed in each sample of a subset of the test samples as compared to the reference samples;
(iv) determining, for each said shared splice variant, one or more amino acid sequences that occur in an amino acid translation of the shared splice variant, but not in amino acid translations of corresponding splice variants of the same gene that are transcribed in the reference samples; and
(v) predicting HLA binding of the one or more shared amino acid sequences, or part thereof, to identify the one or more shared amino acid sequences as one or more shared candidate antigens.

Candidate Antigen/Antigens

The method as defined herein may involve identifying one or more shared candidate antigens for characterising and/or treating of a medical condition.

The term “candidate antigen” as used herein refers to a polypeptide that is predicted to be capable of inducing an immune response in an animal or a nucleic acid (such as an RNA transcript or mRNA) that is predicted to be encodes a polypeptide that is capable of inducing an immune response in an animal. The candidate antigen may be further tested using various techniques such as CyTOF which verifies the candidate antigen as an antigen (i.e. a polypeptide that is capable of inducing an immune response in an animal or a nucleic acid such as an RNA transcript or mRNA) or encodes a polypeptide that is capable of inducing an immune response in an animal.

In some embodiments, the candidate antigen is a HLA binding peptide. In some embodiments, the candidate antigen is a HLA binding peptide which is immunogenic.

In some embodiments, the candidate antigen is a splice variant or a splice variant antigen. The splice variant or splice variant antigen may be a HLA binding peptide and may be immunogenic.

In some embodiments, the candidate antigen is shared across more than one patient suffering from the medical condition and defines a sub-group of patients suffering from the medical condition. The candidate antigen may therefore be referred to as a “shared candidate antigen”.

In some embodiments, the medical condition as defined herein is associated with the expression of the one or more candidate antigens.

The term “splice variant” as used herein may refer to different mRNA molecules which are a result of differential splicing from the same initial pre-mRNA sequence transcribed from a locus, based upon the inclusion or exclusion of specific exon or intron sequences from the initial pre-mRNA transcript sequence. Each separate splice variant may correlate to a specific polypeptide, based on the amino acid sequence encoded by the processed mRNA.

The term “splice variant” may also refer to a polypeptide encoded by a splice variant of an mRNA transcribed from a locus (also known as an isoform). A single locus may therefore encode multiple protein (or polypeptide) splice variants (or isoforms).

A splice variant may be a nucleic acid (such as an RNA transcript or mRNA) or a polypeptide. The term splice variant may also refer to a fragment of a splice variant nucleic acid or polypeptide.

The term “alternative splicing event”, as used herein, designates any sequence variation existing between two polynucleotides arising from the same gene or the same pre-mRNA by alternative splicing. This term also refers to polynucleotides, including splicing isoforms or fragments thereof, comprising said sequence variation. Said sequence variation may be characterized by an insertion or deletion of at least one exon or part of an exon. The term “alternative splicing events” may also encompass skipped exon events, mutually exclusive events (or mutually exclusive exons), alternative 3′ splice sites, alternative 5′ splice sites, or intronic retention events.

The terms “peptide”, “polypeptide” and “protein” are used interchangeably and include any polymer of amino acids (dipeptide or greater) linked through peptide bonds or modified peptide bonds. The polypeptides of the invention may comprise non-peptidic components, such as carbohydrate groups. Carbohydrates and other non-peptidic substituents may be added to a polypeptide by the cell in which the polypeptide is produced, and will vary with the type of cell. Polypeptides are defined herein, in terms of their amino acid backbone structures; substituents such as carbohydrate groups are generally not specified, but may be present nonetheless.

The term “polynucleotide” or “nucleic acid” as used herein designates mRNA, RNA, cDNA or DNA. The term typically refers to polymeric forms of nucleotides of at least 10 bases in length, either ribonucleotides or deoxynucleotides or a modified form of either type of nucleotide. The term includes single and double stranded forms of DNA.

Cancer

The medical condition as referred to herein can be a cancer. The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. By “non-metastatic” is meant a cancer that remains at the primary site and has not penetrated into the lymphatic or blood vessel system or to tissues other than the primary site. The term “metastatic cancer” refers to cancer that has spread or is capable of spreading from one part of the body to another. Generally, a non-metastatic cancer is any cancer that is a Stage 0, I, or II cancer, and occasionally a Stage III cancer. A metastatic cancer, on the other hand, is usually a stage IV cancer.

The term “cancer” includes but is not limited to, breast cancer, large intestinal cancer, lung cancer, small cell lung cancer, gastric (stomach) cancer, liver cancer, blood cancer, bone cancer, pancreatic cancer, skin cancer, head and/or neck cancer, cutaneous or intraocular melanoma, uterine sarcoma, ovarian cancer, rectal or colorectal cancer, anal cancer, colon cancer, fallopian tube carcinoma, endometrial carcinoma, cervical cancer, vulval cancer, squamous cell carcinoma, vaginal carcinoma, Hodgkin's disease, non-Hodgkin's lymphoma, esophageal cancer, small intestine cancer, endocrine cancer, thyroid cancer, parathyroid cancer, adrenal cancer, soft tissue tumour, urethral cancer, penile cancer, prostate cancer, chronic or acute leukemia, lymphocytic lymphoma, bladder cancer, kidney cancer, ureter cancer, renal cell carcinoma, renal pelvic carcinoma, CNS tumour, glioma, astrocytoma, glioblastoma multiforme, primary CNS lymphoma, bone marrow tumour, brain stem nerve gliomas, pituitary adenoma, uveal melanoma (also known as intraocular melanoma), testicular cancer, oral cancer, pharyngeal cancer or a combination thereof.

In some embodiments, the cancer is gastric cancer, head & neck cancer, colorectal cancer or hepatocellular cancer. In some embodiments, the cancer is gastric cancer or colorectal cancer.

In some embodiments, the cancer is gastric cancer. In some embodiments, the cancer is head and/or neck cancer. In some embodiments, the cancer is colorectal cancer. In some embodiments, the cancer is hepatocellular cancer. In some embodiments, the cancer is breast cancer.

In some embodiments, the cancer is one that is characterised by the expression of one or more shared antigens. The cancer may be found in any location of the body, but is defined by the expression of the one or more shared antigens.

In some embodiments, the cancer is a metastatic cancer. The metastatic cancer may be found in different locations of the body but is characterised by the expression of the one or more shared antigens.

The identification of one shared antigen in a particular cancer type (e.g. gastric cancer) may help to characterise other cancer types (e.g. head and neck or colon cancer) that are associated with the expression of the same shared antigen. This may help development of diagnostic tests or treatments across the different cancer types that are associated with the expression of the shared antigen.

MAP/Microtubule Affinity-Regulating Kinase 3 (MARK3)

In some embodiments, the medical condition is cancer and the shared candidate antigen identified for characterising and/or treating the medical condition is a MARK3 splice variant. In other words, the shared candidate antigen may be identified for characterising and/or treating a subgroup of cancer patients suffering from a MARK3-specific cancer, wherein the MARK3-specific cancer is associated with the expression of the MARK3 splice variant. The cancer may be located at any position of the body, but is defined by the expression of the MARK3 splice variant.

In some embodiments, the MARK3 splice variant comprises a peptide having the sequence of RNMSFRFIK (SEQ ID NO: 1), or encode a peptide having the sequence of RNMSFRFIK (SEQ ID NO: 1).

In some embodiments, the MARK3 splice variant comprises a peptide having at least 80% (or at least 88%) sequence identity to SEQ ID NO: 1, or encodes a peptide having at least 80% sequence (or at least 88%) identity to SEQ ID NO: 1.

The MARK3 splice variant may comprise one or more exons shown in the Table 1 in Example 4.

In some embodiments, the MARK3 splice variant (nucleic acid) comprises exon 24. In some embodiments, the MARK3 splice variant comprises exons 23, 24, 25 and 26. In some embodiments, the MARK3 splice variant comprises exons 23, 24 and 26.

In some embodiments, the method as disclosed herein comprises determining the level of a splice variant corresponding to isoform 1 of MARK3 (i.e. ENST00000429436.2, ENST00000335102.5 or ENST00000554627.1).

In some embodiments, the method as disclosed herein comprises determining the level of a splice variant corresponding to isoform 3 of MARK3 (i.e. ENST00000440884.3).

The term isolated as used herein means altered “by the hand of man” from its natural state; i.e., if it occurs in nature, it has been changed or removed from its original environment, or both. The MARK3 splice variant as disclosed herein may be an isolated MARK3 splice variant.

Neuroblastoma Breakpoint Family Member 9 (NBPF9)

In some embodiments, the medical condition is cancer and the shared candidate antigen identified for characterising and/or treating the medical condition is a NBPF9 splice variant. In other words, the shared candidate antigen may be identified for characterising and/or treating a subgroup of cancer patients suffering from a NBPF9-specific cancer, wherein the NBPF9-specific cancer is associated with the expression of the NBPF9 splice variant. The cancer may be located at any position of the body, but is defined by the expression of the NBPF9 splice variant.

The NBPF9 splice variant may be due to an intron retention event that results in the retention an intron (Chr1: 144826287:144826932:+), resulting in transcripts that contain the exon (chr1:144826235:144827105:+).

In some embodiments, the NBPF9 splice variant comprises a peptide having the sequence of SSFYALEEK (SEQ ID NO: 31), or encodes a peptide having the sequence of SSFYALEEK (SEQ ID NO: 31).

In some embodiments, the NBPF9 splice variant comprises a peptide having at least 80% (or at least 88%) sequence identity to SEQ ID NO: 31, or encodes a peptide having at least 80% sequence (or at least 88%) identity to SEQ ID NO: 31.

The NBPF9 splice variant as disclosed herein may be an isolated NBPF9 splice variant.

Par-3 Family Cell Polarity Regulator (PARD3)

In some embodiments, the medical condition is cancer and the shared candidate antigen identified for characterising and/or treating the medical condition is a PARD3 splice variant. In other words, the shared candidate antigen may be identified for characterising and/or treating a subgroup of cancer patients suffering from a PARD3-specific cancer, wherein the PARD3-specific cancer is associated with the expression of the PARD3 splice variant. The cancer may be located at any position of the body, but is defined by the expression of the PARD3 splice variant.

The PARD3 splice variant may be due to an alternative usage of 5′ splice site that results in transcripts that contain the exons (chr10:34625127:34625171:− and chr10:34626206:34626354:−).

In some embodiments, the PARD3 splice variant comprises a peptide having the sequence of SQLDFVKTRK (SEQ ID NO: 32), or encodes a peptide having the sequence of SQLDFVKTRK (SEQ ID NO: 32).

In some embodiments, the PARD3 splice variant comprises a peptide having at least 80% (or at least 88%) sequence identity to SEQ ID NO: 32, or encodes a peptide having at least 80% sequence (or at least 88%) identity to SEQ ID NO: 32.

The PARD3 splice variant as disclosed herein may be an isolated PARD3 splice variant.

Zinc Finger CCCH-Type Containing, Antiviral 1 (ZC3HAV1)

In some embodiments, the medical condition is cancer and the shared candidate antigen identified for characterising and/or treating the medical condition is a ZC3HAV1 splice variant. In other words, the shared candidate antigen may be identified for characterising and/or treating a subgroup of cancer patients suffering from a ZC3HAV1-specific cancer, wherein the ZC3HAV1-specific cancer is associated with the expression of the ZC3HAV1 splice variant. The cancer may be located at any position of the body, but is defined by the expression of the ZC3HAV1 splice variant.

The ZC3HAV1 splice variant may be due to an alternative usage of 5′ splice site that results in transcripts that contain the exons (chr7:138763298:138763399:−, and chr7: 138763850:138764989:−).

In some embodiments, the ZC3HAV1 splice variant comprises a peptide having the sequence of LTMAVKAEK (SEQ ID NO: 33), or encodes a peptide having the sequence of LTMAVKAEK (SEQ ID NO: 33).

In some embodiments, the ZC3HAV1 splice variant comprises a peptide having at least 80% (or at least 88%) sequence identity to SEQ ID NO: 33, or encodes a peptide having at least 80% sequence (or at least 88%) identity to SEQ ID NO: 33.

The ZC3HAV1 splice variant as disclosed herein may be an isolated ZC3HAV1 splice variant.

YY1 Associated Factor 2 (YAF2)

In some embodiments, the medical condition is cancer and the shared candidate antigen identified for characterising and/or treating the medical condition is a YAF2 splice variant. In other words, the shared candidate antigen may be identified for characterising and/or treating a subgroup of cancer patients suffering from a YAF2-specific cancer, wherein the YAF2-specific cancer is associated with the expression of the YAF2 splice variant. The cancer may be located at any position of the body, but is defined by the expression of the YAF2 splice variant.

The YAF2 splice variant may be due to an alternative usage of 3′ splice site that results in transcripts that contain the exons (chr12:42604350:42604421:−, and chr12: 42631401:42631526:−).

In some embodiments, the YAF2 splice variant comprises a peptide having the sequence of VIVSASRTK (SEQ ID NO: 34), or encodes a peptide having the sequence of VIVSASRTK (SEQ ID NO: 34).

In some embodiments, the YAF2 splice variant comprises a peptide having at least 80% (or at least 88%) sequence identity to SEQ ID NO: 34, or encodes a peptide having at least 80% sequence (or at least 88%) identity to SEQ ID NO: 34.

The YAF2 splice variant as disclosed herein may be an isolated YAF2 splice variant.

Calcium/Calmodulin-Dependent Protein Kinase Kinase 1 (CAMKK1)

In some embodiments, the medical condition is cancer and the shared candidate antigen identified for characterising and/or treating the medical condition is a CAMKK1 splice variant. In other words, the shared candidate antigen may be identified for characterising and/or treating a subgroup of cancer patients suffering from a CAMKK1-specific cancer, wherein the CAMKK1-specific cancer is associated with the expression of the CAMKK1 splice variant. The cancer may be located at any position of the body, but is defined by the expression of the CAMKK1 splice variant.

The CAMKK1 splice variant may be due to an exon skip/inclusion event that results in the skipping of an exon (chr17:3784921-3784942:−), resulting in transcripts that contain the exons (chr17:3785822-3785858:− and chr17:3783640-3783728:−).

In some embodiments, the CAMKK1 splice variant comprises a peptide having the sequence of VTSPSRRSK (SEQ ID NO: 35), or encodes a peptide having the sequence of VTSPSRRSK (SEQ ID NO: 35).

In some embodiments, the CAMKK1 splice variant comprises a peptide having at least 80% (or at least 88%) sequence identity to SEQ ID NO: 35, or encodes a peptide having at least 80% sequence (or at least 88%) identity to SEQ ID NO: 35.

The CAMKK1 splice variant as disclosed herein may be an isolated CAMKK1 splice variant.

Leucine-Rich Repeat Protein 1 (LRR1)

In some embodiments, the medical condition is cancer and the shared candidate antigen identified for characterising and/or treating the medical condition is a LRR1 splice variant. In other words, the shared candidate antigen may be identified for characterising and/or treating a subgroup of cancer patients suffering from a LRR1-specific cancer, wherein the LRR1-specific cancer is associated with the expression of the LRR1 splice variant. The cancer may be located at any position of the body, but is defined by the expression of the LRR1 splice variant.

The LRR1 splice variant may be due to an exon skip/inclusion event that results in the skipping of an exon (chr14:50074118-50074839:+(SEQ ID NO: 42)), resulting in transcripts that contain the exons (chr14:50069088-50069186:+ and chr14:50080974-50081389:+).

In some embodiments, the LRR1 splice variant comprises a peptide having the sequence of SLPRFGYRK (SEQ ID NO: 36), or encodes a peptide having the sequence of SLPRFGYRK (SEQ ID NO: 36).

In some embodiments, the LRR1 splice variant comprises a peptide having at least 80% (or at least 88%) sequence identity to SLPRFGYRK (SEQ ID NO: 36), or encodes a peptide having at least 80% sequence (or at least 88%) identity to SLPRFGYRK (SEQ ID NO: 36).

In some embodiments, the LRR1 splice variant comprises a peptide having the sequence of SYHSIPSLPRF (SEQ ID NO: 51), or encodes a peptide having the sequence of SYHSIPSLPRF (SEQ ID NO: 51).

In some embodiments, the LRR1 splice variant comprises a peptide having at least 80% (or at least 88%) sequence identity to SYHSIPSLPRF (SEQ ID NO: 51), or encodes a peptide having at least 80% sequence (or at least 88%) identity to SYHSIPSLPRF (SEQ ID NO: 51).

The LRR1 splice variant as disclosed herein may be an isolated LRR1 splice variant.

Zinc Finger Protein 670 (ZNF670)

In some embodiments, the medical condition is cancer and the shared candidate antigen identified for characterising and/or treating the medical condition is a ZNF670 splice variant. In other words, the shared candidate antigen may be identified for characterising and/or treating a subgroup of cancer patients suffering from a ZNF670-specific cancer, wherein the ZNF670-specific cancer is associated with the expression of the ZNF670 splice variant. The cancer may be located at any position of the body, but is defined by the expression of the ZNF670 splice variant.

The ZNF670 splice variant may be due to an exon skip/inclusion event that results in the skipping of an exon (chr1:247130997-247131094:−(SEQ ID NO: 45)), resulting in transcripts that contain the exon (chr1:247151423-247151557:− and chr1:247108849-247109129:−).

In some embodiments, the ZNF670 splice variant comprises a peptide having the sequence of SCVSPSSELK (SEQ ID NO: 37), or encodes a peptide having the sequence of SCVSPSSELK (SEQ ID NO: 37).

In some embodiments, the ZNF670 splice variant comprises a peptide having at least 80% (or at least 88%) sequence identity to SCVSPSSELK (SEQ ID NO: 37), or encodes a peptide having at least 80% sequence (or at least 88%) identity to SCVSPSSELK (SEQ ID NO: 37).

The ZNF670 splice variant as disclosed herein may be an isolated ZNF670 splice variant.

Glutamate Ionotropic Receptor NMDA Type Subunit Associated Protein 1 (GRINA)

In some embodiments, the medical condition is cancer and the shared candidate antigen identified for characterising and/or treating the medical condition is a GRINA splice variant. In other words, the shared candidate antigen may be identified for characterising and/or treating a subgroup of cancer patients suffering from a GRINA-specific cancer, wherein the GRINA-specific cancer is associated with the expression of the GRINA splice variant. The cancer may be located at any position of the body, but is defined by the expression of the GRINA splice variant.

The GRINA splice variant may be due to an intron retention event that results in the removal of an intron (chr8:145065973: 145066412:+) resulting in transcripts that does not contain the intron (chr8:145065860-145065972:+@chr8:145066413-145066541:+).

In some embodiments, the GRINA splice variant comprises a peptide having the sequence of SIRQAFIRK (SEQ ID NO: 38), or encodes a peptide having the sequence of SIRQAFIRK (SEQ ID NO: 38).

In some embodiments, the GRINA splice variant comprises a peptide having at least 80% (or at least 88%) sequence identity to SIRQAFIRK (SEQ ID NO: 38), or encodes a peptide having at least 80% sequence (or at least 88%) identity to SIRQAFIRK (SEQ ID NO: 38).

The GRINA splice variant as disclosed herein may be an isolated GRINA splice variant.

Myeloid Zinc Finger 1 (MZF1)

In some embodiments, the medical condition is cancer and the shared candidate antigen identified for characterising and/or treating the medical condition is a MZF1 splice variant. In other words, the shared candidate antigen may be identified for characterising and/or treating a subgroup of cancer patients suffering from a MZF1-specific cancer, wherein the MZF1-specific cancer is associated with the expression of the MZF1 splice variant. The cancer may be located at any position of the body, but is defined by the expression of the MZF1 splice variant.

The MZF1 splice variant may be due to an intron retention event that results in the retention of an intron (chr19:59,081,895-59,082,360:−), resulting in transcripts that contain the intron retention event (chr19:59081711-59082796:−).

In some embodiments, the MZF1 splice variant comprises a peptide having the sequence of KWPPATETL (SEQ ID NO: 52), or encodes a peptide having the sequence of KWPPATETL (SEQ ID NO: 52).

In some embodiments, the MZF1 splice variant comprises a peptide having at least 80% (or at least 88%) sequence identity to KWPPATETL (SEQ ID NO: 52), or encodes a peptide having at least 80% sequence (or at least 88%) identity to KWPPATETL (SEQ ID NO: 52).

The MZF1 splice variant as disclosed herein may be an isolated MZF1 splice variant.

Reference

A “reference” as referred to herein may be one or more samples that are not affected by a medical condition (e.g., non-cancerous cells) taken from the subject having the medical condition, or one or more samples taken from another subject (e.g. a healthy subject who does not suffer from the medical condition). The reference may also be a pre-determined value or an average value of a measurement of the sample, such as an expression level of a transcript in the sample.

Sample

As used herein, the term “sample” (or “test samples”) includes tissues, cells, body fluids and isolates thereof etc., isolated from a subject, as well as tissues, cells and fluids etc. present within a subject (i.e. the sample is in vivo). Examples of samples include: whole blood, blood fluids (e.g. serum and plasm), lymph and cystic fluids, sputum, stool, tears, mucus, hair, skin, ascitic fluid, cystic fluid, urine, nipple exudates, nipple aspirates, sections of tissues such as biopsy and autopsy samples, frozen sections taken for histologic purposes, archival samples, explants and primary and/or transformed cell cultures derived from patient tissues etc.

The sample may be obtained at one or more time points. Expression levels of a splice variant may optionally be compared with a reference. The reference may be a control sample derived from a person not having the medical condition. One or more control samples may be employed.

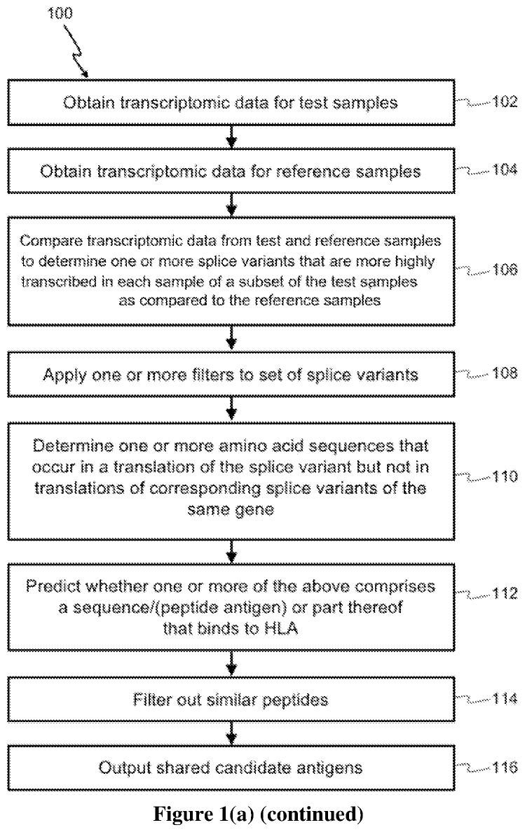

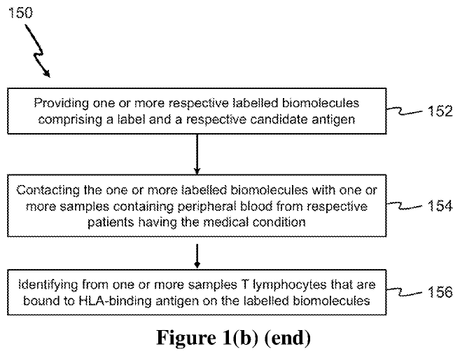

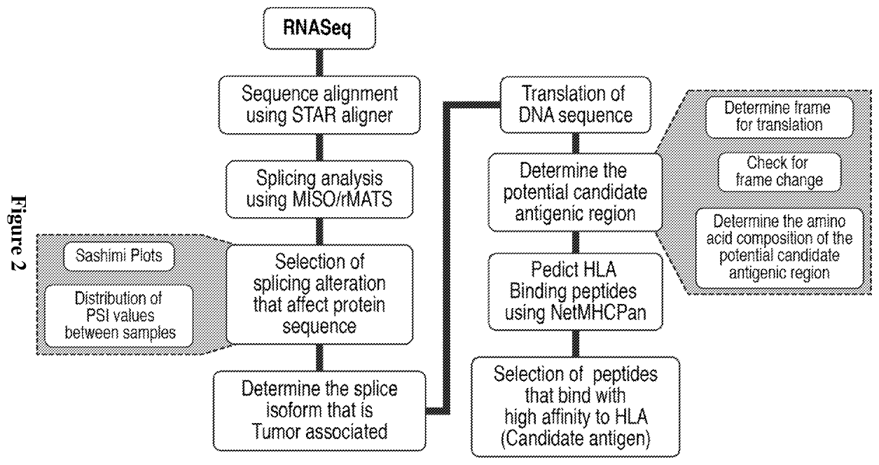

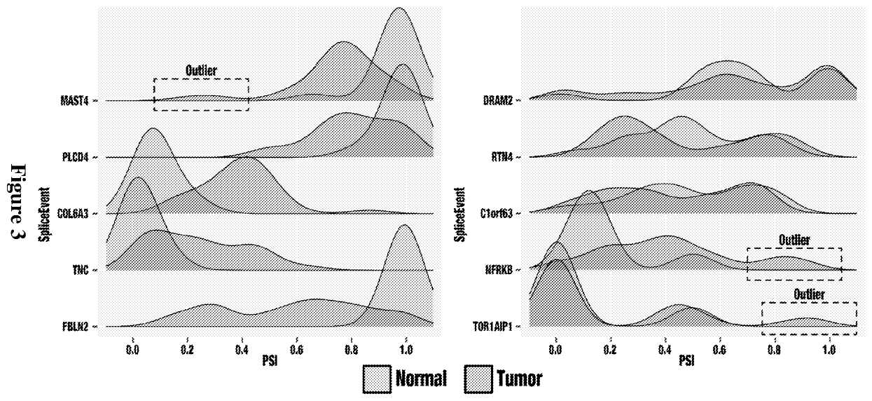

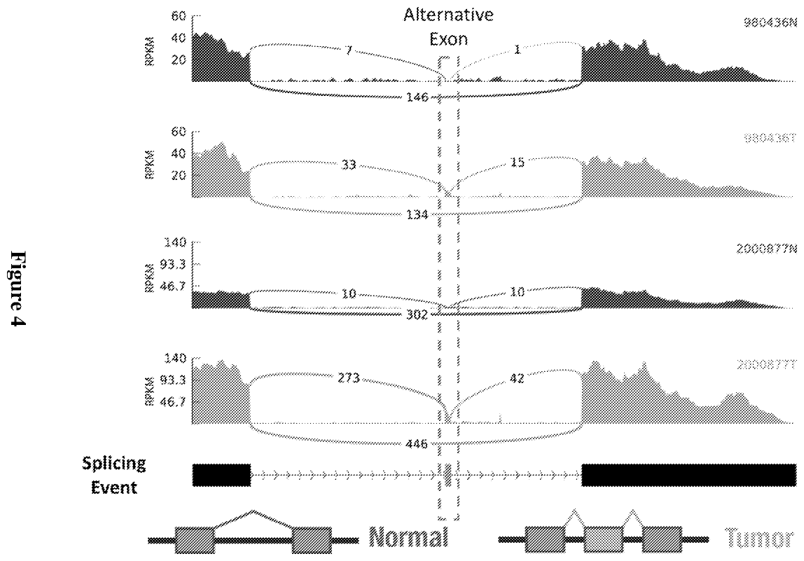

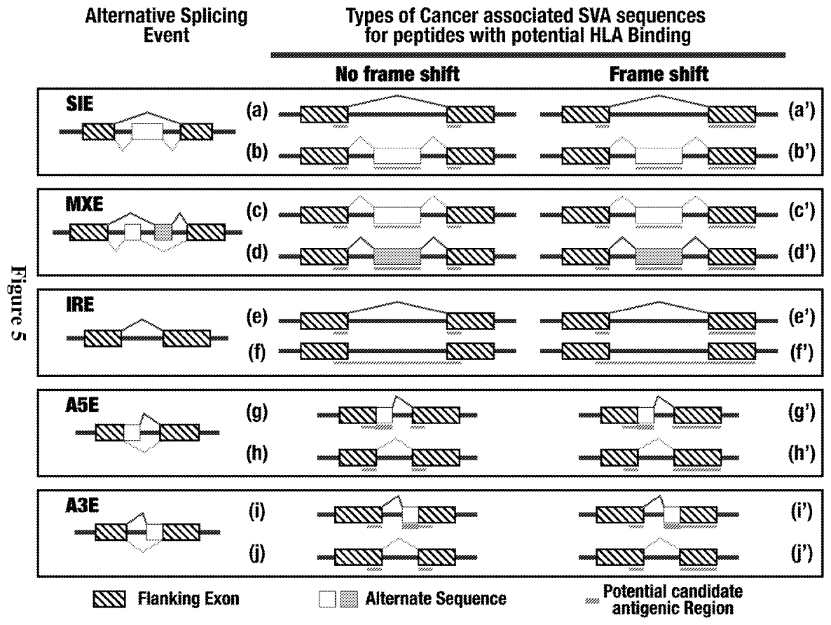

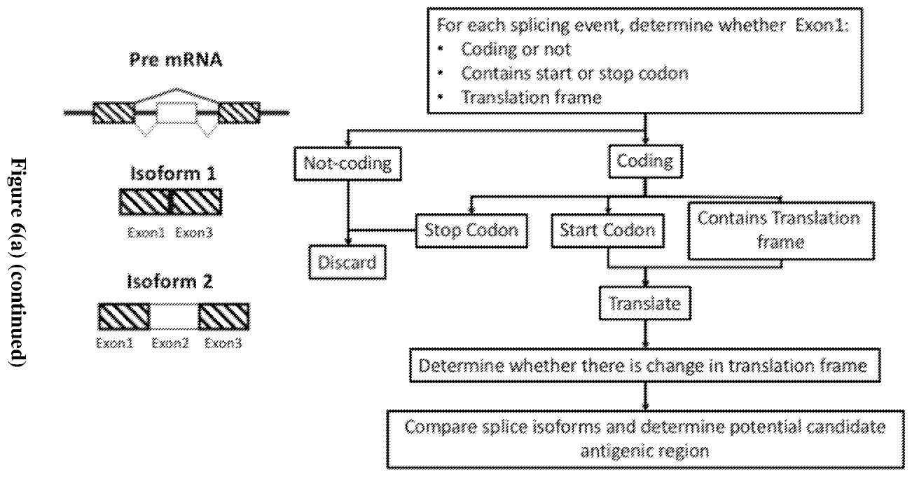

Referring now to FIG. 1(a), a method 100 of identifying one or more shared candidate antigens for characterising and/or treating a medical condition includes a step 102 of obtaining transcriptomic data for test samples from a first cohort of patients having the medical condition. Typically, the method 100 is at least partly, and in some embodiments entirely, performed by at least one processor of one or more computing devices.

In certain embodiments, the first cohort of patients may be selected in accordance with one or more clinical parameters. For example, the one or more clinical parameters may include parameters related to the medical condition (such as disease subtype, for example tumour type, or disease progression status), or HLA subtype.

For example, the transcriptomic data may be sequencing data, such as whole transcriptome shotgun sequencing (WTSS) data (also known as RNA-Seq data). Certain embodiments will be described with reference to WTSS data, but it will be appreciated that other forms of transcriptomic data may be used in other embodiments of the method, such as probe-level or probe-set data from measurement platforms such as exon microarrays, splice junction microarrays, or tiling arrays.

The transcriptomic data may be obtained by sequencing samples from the first cohort of patients, or by retrieving transcriptomic data that has previously been generated from the first cohort of patients. For example, the transcriptomic data may be stored on a computer-readable medium and retrieved via a local bus or over a communications link.

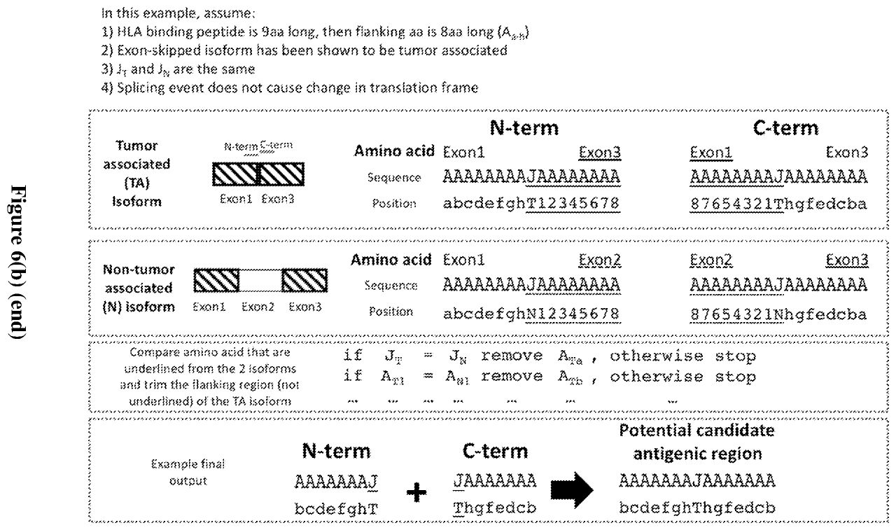

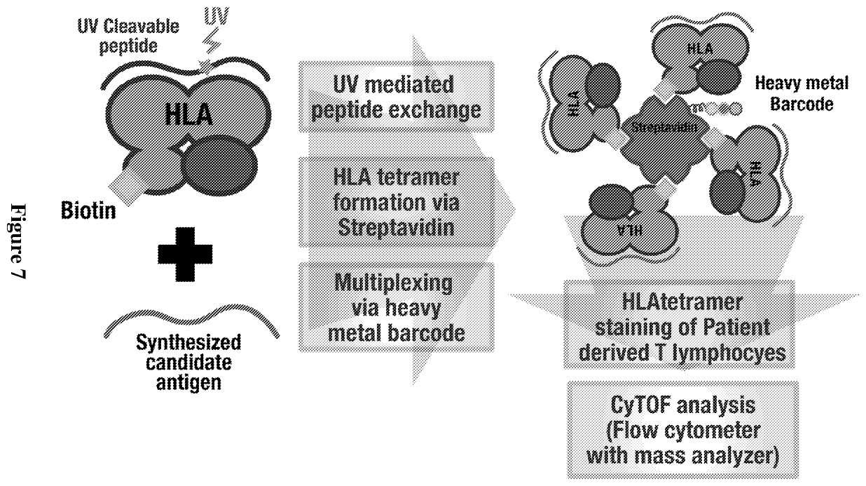

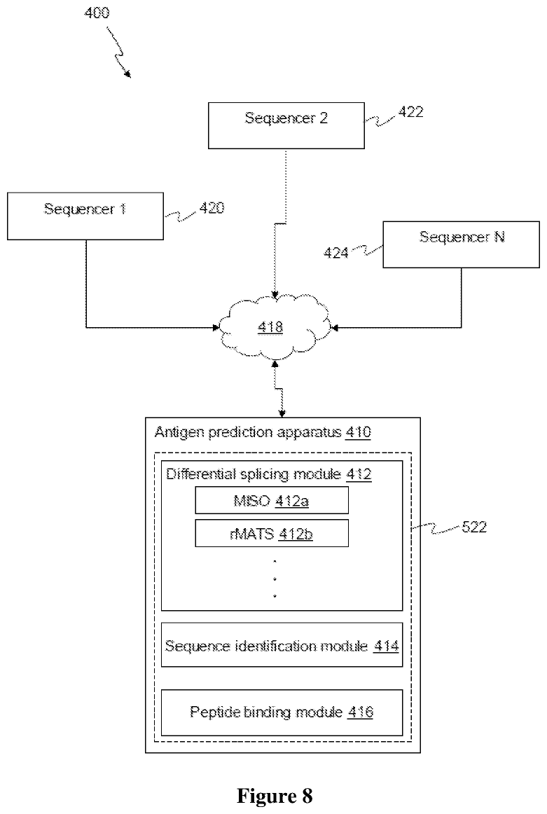

The transcriptomic data may be raw sequence reads or may be data generated by at least one operation performed on the raw sequence reads. For example, the at least one operation may include a pre-processing operation to remove low-quality reads. The at least one operation may also include aligning the sequence reads to one or more reference sequences, such as a reference genome. For example, as shown in the workflow of FIG. 2, the sequence reads may be aligned using a sequence aligner such as STAR (A. Dobin et al (2013) Bioinformatics 29, pages 15-21) or Bowtie (B. Langmead et al (2009) Genome Biology 10:R25), or any like sequence alignment tool. The sequence alignments may be output in a file format such as SAM or BAM, for example.

In some embodiments, the sequence reads may be aligned to splice junction sequences. The splice junction sequences may be obtained based on known or predicted exon-intron boundaries, and/or may be determined by spliced alignment to a reference genome.

The method 100 also includes a step 104 of obtaining reference transcriptomic data for a set of reference samples. The reference samples may be, for example, one or more samples that are not affected by the medical condition (e.g.: non-cancerous, normal, healthy cells) taken from the patient having the medical condition, or one or more samples taken from one or more other subjects who do not suffer from the medical condition.

As for the transcriptomic data of the test samples, the transcriptomic data may be obtained by sequencing the reference samples, or by retrieving transcriptomic data that has previously been generated from the reference samples. Similar pre-processing operations as performed on the transcriptomic data of the test samples (including quality control and sequence read alignment) may be performed on the transcriptomic data of the reference samples.

In some embodiments the transcriptomic data in step 102 and/or step 104 may be obtained from databases or pre-existing datasets. These include, for example, publicly available databases such as GTEx, TCGA, etc.

The method 100 further includes a step 106 of determining, by a comparison of the transcriptomic data to the reference transcriptomic data, one or more first splice variants that are differentially spliced between the test samples and the reference samples. This may be done by determining if the one or more splice variants are more highly transcribed in each sample of a subset of the test samples as compared to the reference samples;

For example, as shown in the workflow of FIG. 2, a differential splicing analysis may be performed using a tool such as MISO (Y. Katz et al (2010) Nature Methods 7(12), pages 1009-1015) or rMATS (S. Shen et al (2014) Proc Nat Acad Sci 111 (51) E5593-E5601), though it will be appreciated that a number of other tools for determining differential splicing may be used.

Currently differential splicing analysis using RNA-Seq uses sequencing read density to determine the isoforms (isoform level), exons (exon level) or splice junctions (junctional level) that are expressed or utilized in the cell. At the isoform level, all the sequencing reads mapping to one gene is used to determine the exon composition of the isoform that is expressed in the cell. This may then be compared to a reference to determine differential expression between tumour and normal samples. This may be highly challenging as there may be multiple isoforms for each gene and different isoforms may be concurrently expressed in each cell. At the exon level, splicing analysis may involve determining whether there is inclusion or skipping of particular sequences (introns[IRE], exons[SIE,MXE) or parts of exons [A5E, A3E]). This uses sequencing read density around exons and their corresponding junction to determine whether there is inclusion or exclusion. Differential splicing analysis is done by a comparison between tumour and normal samples. At the junctional level, splicing analysis may involve determining how sequences are joined together. Only sequencing reads mapping to splice junctions are considered. This may then be compared to a reference to determine differential usage of splice junctions between tumour and normal samples.

Differential splicing analysis in the method disclosed herein is undertaken at the exon level, wherein the differential splicing analysis may include determining a “percentage spliced in” (PSI or Ψ) score, for each splice variant, from the density of the sequence reads that map to the splice variant exons.

For example, in the case of a splice variant that includes an additional exon, PSI may be estimated for the splice variant using Ψ=IR/IR+ER, where IR is the number of inclusion reads (reads that map to the additional exon as well as to its splice junctions with the exons adjacent to it) and ER is the number of exclusion reads (reads that map to the splice junction between the adjacent exons). IR and ER may be normalised according to methods known in the art. This is done for both the test samples and the reference samples, and differential splicing may be determined by, for example, computing a difference ΔΨ between the two Ψ values and comparing the difference to a threshold, and/or by using another technique such as computation of a Bayes factor (e.g., using the Savage-Dickey density ratio as in MISO) and comparing the Bayes factor to a threshold. In some embodiments, a splice variant may be called as differentially spliced if |ΔΨ|exceeds a threshold and the Bayes factor exceeds another threshold, e.g. |ΔΨ|>0.2 and Bayes factor greater than 20.

In some embodiments, instead of a PSI score, an alternative measure of differential splicing may be determined, such as a “percent spliced out” score. For example, the percent spliced out score may be determined according to (1−Ψ)=ER/(IR+ER).

In some embodiments, one or more additional filtering operations 108 may be applied to the set of splice variants called as being differentially spliced. For example, a quality control operation may be performed by examining Sashimi plots (https://www.biorxiv.org/content/10.1101/002576v1) of the read mappings to the reference sequence(s), and removing any splice variants that do not satisfy predetermined criteria.

For example, the quality control operation may include analysing distributions of PSI in splicing events in the test samples (e.g., tumour samples) to identify differential splicing events. Any such differential splicing event may also be examined further to determine whether it is, for example, an exon skipping or inclusion event.

In general, splicing events from samples that are unaffected by the medical condition will have a relatively narrow PSI distribution. Due to the heterogeneity of tumour samples, splicing events from tumour samples may also have similar PSI values to those in reference (normal) samples, but tumour samples may have, in addition, splicing events that are different from those in reference samples. In the case of cancer, therefore, tumour samples may be analysed to check whether they have PSI values that are different from those of the reference sample. These are referred to as ‘outliers’ (FIG. 3).

The distribution of PSI values of all samples (both test/tumour and reference/normal) are compared. Splice events are selected based on two criteria: 1) tumour samples having PSI values that are different from those of normal samples, and 2) a sufficient number of tumour samples show splicing such as an exon skipping or inclusion event that differs from the normal patients. This last criterion allows shared tumour associated splicing events to be identified.

In one example, as part of the filtering operations 108, Sashimi plots may be used to examine the sequencing reads that map to the junctions (FIG. 4). For example, in cases where there is alternate usage of an exon (two junctions for exon inclusion and one junction for a skipped exon) that resides in the middle of the transcript, it is expected for RNA-Seq data that both junctions would have similar counts. If there are very skewed counts for only one junction in an exon inclusion, it is doubtful whether a splice event has actually occurred. In certain embodiments, the filtering operation 108 may include requiring a threshold number of counts at splice junctions (e.g., more than five counts) to be confident that there are differences in terms of splicing.

In another example, the filtering operations may include selecting splice variants that are more frequently found in the test samples than the reference samples. In a further example, the filtering operations may include selecting splice variants that are found in at least a threshold number (e.g., at least 2, at least 3, at least 4, or at least 5) or at least a threshold proportion of the test samples (e.g., at least 1%, at least 2%, at least 5%, at least 10%, at least 15%, or at least 20% of the test samples). This would imply that the splice variants are shared in a subset of the test samples. In a yet further example, the filtering operations may include selecting splice variants that are found in the test samples but not in the reference samples.

In some embodiments, the subset comprises more than a threshold number or more than a threshold percentage of the test samples.

In some embodiments, the filtering operations 108 may include identifying splice variants that undergo a change in reading frame. Advantageously, such splice variants present novel protein sequences and/or produce longer coding sequence, and therefore lead to a larger number of candidate peptides.

In some embodiments, the method further includes determining for each said splice variant, prior to step (iv), whether there is a change in reading frame in the first splice variant relative to the one or more corresponding splice variants of the same gene.

The method 100 includes a step 110 of determining, for each splice variant, one or more amino acid sequences that occur in an amino acid translation of the splice variant, but not in amino acid translations of corresponding splice variants of the same gene that are transcribed in the reference samples.

In some embodiments, step (iv) includes determining non-overlapping nucleotide sequence between the splice variant and corresponding splice variants of the same gene.

These sequences represent potential candidate antigenic regions which may be used to determine whether they comprise HLA-binding peptides.

Examples of candidate antigenic regions for different types of alternative splicing event are shown in FIG. 5. Different types of candidate antigenic regions may be generated depending on whether a frame shift occurs or not. In each of the examples in FIG. 5, the underlined portion constitutes the potential candidate antigenic region.

For example, for an exon skip event with no frame shift (as at FIG. 5(a)), a candidate antigenic region may include a sequence (indicated as a portion underlining parts of the two exons) that spans the junction between the flanking exons either side of the junction. On the other hand, if the exon skip event causes a frame shift (as in FIG. 5(a′)), the candidate antigenic region may include additional sequence that covers the entirety of the 3′ flanking exon.

In another example, as shown in FIG. 5(i), the alternative splicing event may be the presence of an alternative 3′ splice site, and without a frame shift, the candidate antigenic region spans part of each exon, with an additional portion spanning the additional sequence that is transcribed due to the alternative 3′ splice site. If a frame shift is present as in FIG. 5(i′), then the candidate antigenic region may span the entirety of the 3′ exon, for example.

Accordingly, it can be seen that it is advantageous to select alternative splicing events that cause frame shifts, because these present novel protein sequences and/or might generate longer candidate antigenic regions, with concomitantly greater opportunities to locate potential HLA-binding peptides that are shared among a subset of patients.

The means for determining whether a splicing event results in an altered amino acid sequence that might yield potential antigenic regions is shown schematically in FIG. 6 (a). Step 110 may include:
determining whether a splice event is coding or not;
determining the open reading frame of exon 1 (this could include, for example, determining whether there are start codons);
translating and determining whether there are any changes to frame (this applies to each of IRE, SIE, MXE, A5E and A3E splicing events); and
comparing splice isoforms and determining potential candidate antigenic regions.

Potential candidate antigenic regions are composed of the flanking regions of the splice event and may contain inclusion of sequences (for example, inclusion of the middle exon in a SIE event). For each splicing event, there are two flanking regions, an N-term and a C-term. The length of the flanking region is affected by the length of HLA binding peptide that is being predicted. The maximal length of the flanking region is the length of HLA binding peptide minus 1. Additionally, the C-term flanking region may comprise the entire sequence of the exon if the splicing alteration leads to change in translation frame. The means for determining the amino acid sequence of the potential candidate antigenic region of a splicing event is shown in FIG. 6 (b).

As shown in FIG. 6(b), for each splicing event, the method for comparing splice isoforms and determining potential candidate antigenic region may comprise:
determining the amino acid composition of the flanking regions;
determining whether splicing event leads to the inclusion of sequences and
including these amino acid sequence into the potential candidate antigenic region; and
joining all these amino acid sequences together to obtain the potential candidate antigenic region.

Step 112 of method 100 may include predicting, from an amino acid translation of the candidate antigenic region, one or more candidate HLA-binding peptides using a binding prediction tool such as NetMHCPan (V. Jurtz et al (2017) J Immunol 199(9):3360-3368). NetMHCpan prediction of peptide HLA binding is based on an algorithm that ranks the predicted affinity of an unknown peptide by comparing its sequences to experimentally determined peptide bound to HLA. Exemplary parameters and cut-offs for NetMHCpan are as follows: 8-11 amino acid peptides used to predict HLA binding; and high affinity binding to HLA (based on top 0.5% rank).

In some embodiments, the method 100 may include a step 114 of filtering out peptides that are similar to each other, to reduce redundancy in the candidate set of peptides.

For example, if the motif of a HLA is: Arg anchor residues at position 7 of the 9aa peptide, then it may be the case that there would be peptides of length 8-11aa that might all bind this HLA molecule and they would have different binding affinities. Filtering of peptides may be done by examining these “related” peptides (similar region of the protein) and keeping the one with the highest predicted binding affinity.

At step 116, the candidate antigens (HLA binding peptides) that remain following filtering operation 114 are output. For example, a listing of candidate antigens may be output as a text file or a similar format. In some embodiments, step 116 may include ranking of all remaining peptides based on their predicted presentation on HLA molecules and using this ranking to select a set of candidate antigens to be used in the identification of antigen-specific T lymphocytes.

The method as defined herein may comprise verifying or testing HLA binding of the one or more shared amino acid sequences to identify the one or more shared amino acid sequences as a shared candidate antigens. The method as defined herein may comprise a step of verifying or testing whether the predicted HLA-binding peptides can bind to HLA molecules or is bound to HLA molecules. This may be done using HLA-peptide binding assays or HLA peptide elution experiments. Such assays are well-known by a person skilled in the art.

The method as defined herein may comprise a further step of determining the immunogenicity of the shared antigen by verifying or testing whether the predicted HLA-binding peptides bind to T lymphocytes. This may be done by: 1) identifying T lymphocytes that bind specifically to the one or more shared candidate antigens that are predicted to bind HLA, 2) functional characterization of T lymphocytes, for example detection of IFN-γ secretion. Such assays are well-known by a person skilled in the art and provide validation that the predicted HLA-binding peptide is immunogenic and can be recognized by T lymphocytes. Such further testing may help to identify the one or more shared candidate antigens as shared antigens.

Antigen-Specific T-Lymphocyte and Shared Antigen-T Lymphocyte Pair

Disclosed herein is a method of identifying a shared antigen-T lymphocyte pair, the method comprising:
a) identifying a shared candidate antigen according to a method as defined herein;
providing one or more respective labelled biomolecules comprising a label and a peptide comprising the shared candidate antigen;
b) contacting the one or more labelled biomolecules with one or more samples containing peripheral blood from patients having the medical condition; and

c) identifying, from the one or more samples, T lymphocytes that are bound to said labelled biomolecules, so as to identify a shared antigen-T lymphocyte pair.

The identification of the shared antigen-T lymphocyte pair identifies the shared candidate antigen as a shared antigen.

Provided herein is a shared antigen-T lymphocyte pair identified according to a method as defined herein.

Provided herein is a shared antigen-T lymphocyte pair identified according to a method as defined herein, wherein the shared antigen is a MARK3 splice variant, the HLA subtype is HLA-A11, and the T lymphocyte binds to the shared MARK3 antigen.

Provided herein is a shared antigen-T lymphocyte pair identified according to a method as defined herein, wherein the shared antigen is a NBPF9 splice variant, the HLA subtype is HLA-A11, and the T lymphocyte binds to the shared NBPF9 antigen.

Provided herein is a shared antigen-T lymphocyte pair identified according to a method as defined herein, wherein the shared antigen is a PARD3 splice variant, the HLA subtype is HLA-A11, and the T lymphocyte binds to the shared PARD3 antigen.

Provided herein is a shared antigen-T lymphocyte pair identified according to a method as defined herein, wherein the shared antigen is a ZC3HAV1 splice variant, the HLA subtype is HLA-A11, and the T lymphocyte binds to the shared ZC3HAV1 antigen.

Provided herein is a shared antigen-T lymphocyte pair identified according to a method as defined herein, wherein the shared antigen is a YAF2 splice variant, the HLA subtype is HLA-A11, and the T lymphocyte binds to the shared YAF2 antigen.

Provided herein is a shared antigen-T lymphocyte pair identified according to a method as defined herein, wherein the shared antigen is a CAMKK1 splice variant, the HLA subtype is HLA-A11, and the T lymphocyte binds to the shared CAMKK1 antigen.

Provided herein is a shared antigen-T lymphocyte pair identified according to a method as defined herein, wherein the shared antigen is a LRR1 splice variant, the HLA subtype is HLA-A11 or HLA-A24, and the T lymphocyte binds to the shared LRR1 antigen.

Provided herein is a shared antigen-T lymphocyte pair identified according to a method as defined herein, wherein the shared antigen is a ZNF670 splice variant, the HLA subtype is HLA-A11, and the T lymphocyte binds to the shared ZNF670 antigen.

Provided herein is a shared antigen-T lymphocyte pair identified according to a method as defined herein, wherein the shared antigen is a GRINA splice variant, the HLA subtype is HLA-A11, and the T lymphocyte binds to the shared GRINA antigen.

Provided herein is a shared antigen-T lymphocyte pair identified according to a method as defined herein, wherein the shared antigen is a MZF1 splice variant, the HLA subtype is HLA-A24, and the T lymphocyte binds to the shared MZF1 antigen.

Disclosed herein is a method for identifying T lymphocytes that bind specifically to one or more shared candidate antigens identified herein, comprising:
(a) providing one or more respective labelled biomolecules comprising a label and a respective candidate antigen;
(b) contacting the one or more labelled biomolecules with one or more samples containing peripheral blood from respective patients having the medical condition; and
(c) identifying, from the one or more samples, T lymphocytes that are bound to said labelled biomolecules.

Referring now to FIG. 1(b), a method 150 for identifying antigen-specific T lymphocytes will be described. It will be appreciated that the antigen identification method described above with reference to FIG. 1(a) is an essentially entirely in silico process, once transcriptomic data from the first cohort of patients and the reference transcriptomic data is obtained. On the other hand, the method 150 of FIG. 1(b) is an in vitro process that uses the output of the in silico process 100 to identify the antigen-specific T lymphocytes in a second cohort of patients.

At step 152 of method 150, one or more respective labelled biomolecules, each comprising a taggant and a respective candidate antigen, may be provided. In some embodiments, the labelled biomolecules may comprise one or more HLA multimers, for example. Typically, the HLA multimers may be dimers up to decamers. The label allows T lymphocytes that are bound to the biomolecules to be identified and/or isolated (for example, by known flow cytometry technologies such as FACS).

Provided herein is a method of identifying and characterizing T lymphocytes that bind specifically to one or more shared candidate antigens identified according to a method as defined herein, comprising:
(a) providing one or more respective labelled biomolecules comprising a label and a respective shared candidate antigen;
(b) contacting the one or more labelled biomolecules with one or more samples containing peripheral blood from respective a second cohort of patients having the medical condition;
(c) isolating, from the one or more samples, labelled biomolecules that are bound to T lymphocytes;
(d) determining the frequency of occurrence of each shared antigen-T lymphocyte pair and assigning pair to a patient subset;
(e) isolating shared antigen-directed T Lymphocytes by expanding the T lymphocyte fraction from samples containing peripheral blood cells; and
(f) demonstrating that the shared antigen-directed T lymphocyte is capable of recognizing and killing tumour cells containing the shared splice variant transcript identified in the first cohort of patients.

In some embodiments, the expanded T lymphocyte fraction is isolated from a sample containing peripheral blood cells derived from the first, second or any cohort of patients.

Provided herein is a labelled biomolecule comprising a HLA molecule bound to a candidate antigen peptide. The HLA molecule may be biotinylated and bound to a streptavidin molecule. In some embodiments, the labelled biomolecule comprises four biotinylated HLA molecules that are bound to one streptavidin molecule.

The taggant (or label) is a moiety that allows detection using a range of different detection methods well known to a person skilled in the art. Different detection methods may use different taggant moieties; for example, a taggant may comprise a fluorophore, DNA barcode or heavy metals. In some embodiments, the taggant may comprise of single units of such moiety. In some embodiments, the label may be combinations of multiple units of fluorophore or heavy metals. In another embodiment, the label may be combinations of fluorophores, DNA barcodes and heavy metals.

In a preferred embodiment, the taggant is a heavy metal barcode which consists of combinations of different heavy metals such as, for example, lanthanides with different atomic weights. The taggant allows detection, by an apparatus such as a CyTOF machine, of labelled biomolecules bound to a T lymphocyte.

CyTOF (or mass cytometry) utilizes a time of flight mass spectrometer to detect metal tagged antibodies or HLA multimers. The main advantage of using this technique for HLA multimer staining is the capacity to simultaneously detect multiple events in limited samples by the use of heavy metal barcodes.

In some embodiments, there is provided a labelled biomolecule comprising a HLA molecule bound to a shared antigen as defined herein for use in detecting a T lymphocyte that binds specifically to a shared antigen.

In some embodiments, there is provided a labelled biomolecule as defined herein, wherein the shared antigen is a peptide having at least 80% sequence identity to SEQ ID NO:1, or is a nucleic acid encoding a peptide having at least 80% sequence identity to SEQ ID NO:1, and wherein the HLA is HLA-A11.

It is advantageous to provide a library of labelled biomolecules, each of which comprises one of the candidate antigens. To this end, the label of each tagged biomolecule may comprise a barcode, such as a heavy metal barcode, which serves to uniquely identify the respective labelled biomolecules. Accordingly, a sample that contains the library can be contacted with the PBMCs at step 154, such that binding of all of the candidate antigens can be efficiently screened for in a single step.

The PBMCs with which the labelled biomolecules are contacted in step 154 are advantageously obtained from patients having the medical condition who are not part of the first cohort of patients from which transcriptomic data was obtained for the candidate antigen identification method 100. By using an independent group of patients for the T lymphocyte screening process 150, there can be greater confidence that the T lymphocytes that are identified are bound to a candidate antigen that is indeed shared.

At step 156, the method comprises identifying labelled biomolecules that are bound to T lymphocytes. These T lymphocytes bound to one or more labelled biomolecules may comprise one or more sub-sets of T lymphocytes specific for one or more particular splice variant antigens.

In one specific example, as shown schematically in FIG. 7, the method 150 may include HLA tetramer staining of peripheral blood, including preparing HLA tetramers by bacterially expressing and purifying biotin tagged HLA loaded with a UV cleavable peptide. Individual shared candidate antigens are then loaded onto the HLA molecule by UV mediated peptide exchange. Addition of heavy metal barcoded streptavidin causes formation of a tetramer comprising a shared candidate antigen. Peripheral blood (e.g. PBMC) from one or more cohort of patients having the medical condition is stained with a pool of the HLA tetramer and analysis is then done using CyTOF.

At step 156, the method may further comprise immune-profiling the antigen specific T lymphocytes which were identified by the labelled biomolecules. Labelled antibodies are used to reveal the phenotype of these T lymphocytes. For example, if the T lymphocytes bind to a labelled antibody against an exhaustion marker such as PD1, it would mean that the T lymphocytes had previously been activated by antigen and chronically stimulated until they were exhausted.

In some embodiments, it may be advantageous to identify antigen-specific T lymphocytes that show similar immune-phenotypes as T lymphocytes that are specific for commonly encountered viral antigens such as CMV, Flu or EBV. In most people these viral-specific T lymphocytes provide protection from these pathogens. For example, these viral antigen specific T lymphocytes frequently show a central memory phenotype which allows the cells to quickly expand to large numbers when the viral antigen is re-encountered. Finding antigen-specific T lymphocytes that show a similar immune-phenotype as viral-specific T lymphocytes may indicate that the antigen-specific T lymphocytes have activity towards eradicating tumours that express its cognate antigen. Additionally, this would allow prioritization of these antigen-specific T lymphocytes.

At step 156, the method may also comprise the isolation of these antigen-specific T lymphocytes, which were identified by the labelled biomolecules, for the purpose of identifying TCRs that recognize the shared splice variant antigen(s).

Step 152 may involve providing a single labelled biomolecule that comprises a single candidate antigen, and a sample of the labelled biomolecule may then be contacted with peripheral blood mononuclear cells (PBMCs) from one or more patients having the medical condition at step 154 of the method, for the purpose of detecting the presence of the antigen in a cancer patient. If present, the patient may be responsive to immunotherapy targeting the said antigen.

The method may further comprise testing the biological function of the T lymphocytes. The method may comprise testing the biological function of the T lymphocytes in an in vitro assay. Such assays are well-known by a person skilled in the art and may include testing the cell killing activity of the T lymphocytes on cells that are associated with the expression of the one of more candidate antigens.

In some embodiments, the method comprises characterising the T lymphocytes to determine whether they are cytotoxic and/or testing whether the shared candidate antigens are immunogenic.

In some embodiments, the method may further comprise testing the biological function of the candidate antigen and T lymphocytes that are identified to bind to the candidate antigens. Assays for testing the biological function of the T lymphocytes are well-known by a person skilled in the art and may include ELISPOT assays and/or cell killing assays. This provides additional validation that: 1) the antigen(s) is presented on the surface of target cells (such as cancer cells); 2) T lymphocytes can recognize and target the antigen; or 3) T lymphocyte(s) targeting this antigen are functional, for example by performing functions that help eradicate the cancer cell.

The method as defined herein may comprise a further step of verifying the shared nature of the HLA-binding peptides in two different cohorts of patients with a medical condition. The first cohort of patient with medical condition being the “discovery cohort”, used for identifying candidate antigens. The second cohort of patients with a medical condition being the “validation cohort”, used to verify or test whether the predicted HLA-binding peptides bind to T lymphocytes. This is advantageous as there can be greater confidence that the antigen(s) that are identified are indeed shared.

System for Identifying Candidate Antigens

Referring to FIG. 8, an embodiment of a system 400 for identifying shared candidate antigens includes an antigen prediction apparatus 410. The system 400 may also include one or more sequencing platforms 420, 422, 424 that are in communication with antigen prediction apparatus 410 via a network 418.

Antigen prediction apparatus 410 is suitable for at least partly carrying out the method 100, and in particular, may obtain and perform analyses on transcriptomic data from one or more of the sequencing platforms 420, 422, 424 via network 418, and/or from one or more computer-readable media, to generate predictions of one or more candidate HLA-binding peptides.

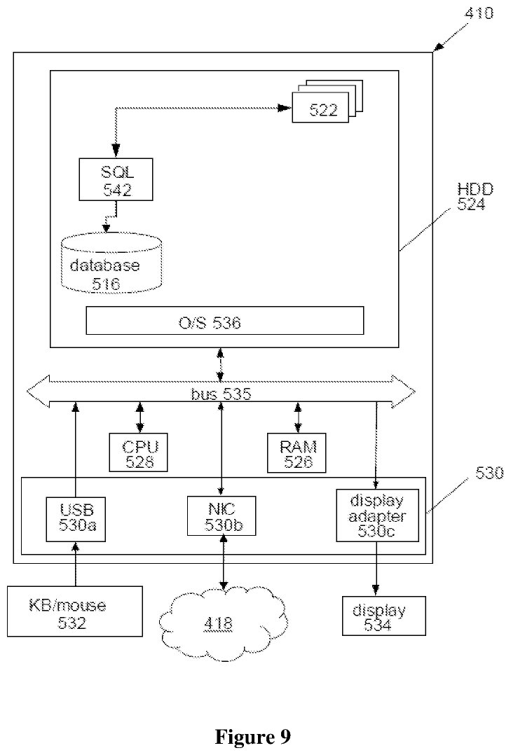

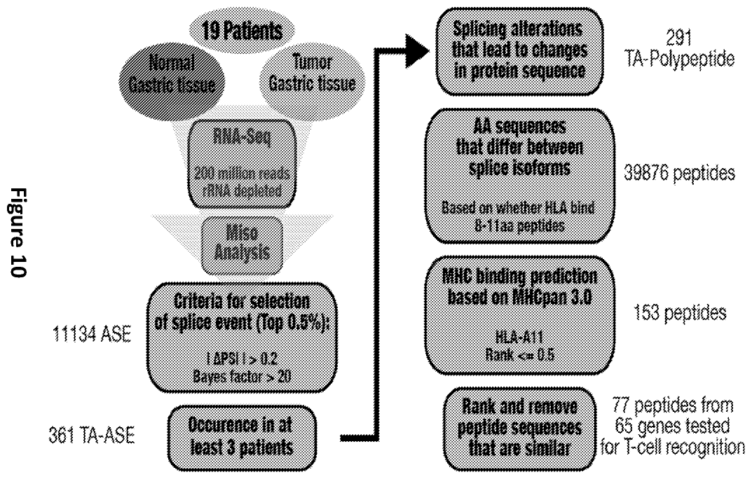

FIG. 9 shows an example computing device 410 that is capable of implementing an antigen prediction apparatus of the system 400. In some embodiments, multiple computing devices 410 may be considered to be a single application server.

The components of the computing device 410 can be configured in a variety of ways. The components can be implemented entirely by software to be executed on standard computer server hardware, which may comprise one hardware unit or different computer hardware units distributed over various locations, which may communicate over a network. Some of the components or parts thereof may also be implemented by application specific integrated circuits (ASICs) or field programmable gate arrays.

In the example shown in FIG. 9, the computing device 410 is a commercially available server computer system based on a 32 bit or a 64 bit Intel architecture, and the processes and/or methods executed or performed by the computing device 410 are implemented in the form of programming instructions of one or more software components or modules 522 stored on non-volatile (e.g., hard disk) computer-readable storage 524 associated with the computing device 410. At least parts of the software modules 522 could alternatively be implemented as one or more dedicated hardware components, such as application-specific integrated circuits (ASICs) and/or field programmable gate arrays (FPGAs).

The computing device 410 includes at least one or more of the following standard, commercially available, computer components, all interconnected by a bus 535:
(a) random access memory (RAM) 526;
(b) at least one computer processor 528, and
(c) external computer interfaces 530:
   (i) universal serial bus (USB) interfaces 530a (at least one of which is connected to one or more user-interface devices, such as a keyboard, a pointing device (e.g., a mouse 532 or touchpad),
   (ii) a network interface connector (NIC) 530b which connects the computer device 410 to a data communications network and/or to external devices; and
   (iii) a display adapter 530c, which is connected to a display device 534 such as a liquid-crystal display (LCD) panel device.

The computing device 410 includes a plurality of standard software modules, including:
(a) an operating system (OS) 536 (e.g., Linux or Microsoft Windows); and
(b) structured query language (SQL) modules 542 (e.g., MySQL, available from http://www.mysql.com), which allow data, such as input transcriptomic data and/or output candidate HLA-binding peptides, to be stored in and retrieved/accessed from an SQL database 516.

Advantageously, the database 516 forms part of the computer readable data storage 524. Alternatively, the database 516 is located remote from the server 410 shown in FIG. 8.

The boundaries between the modules and components in the software modules 522 are exemplary, and alternative embodiments may merge modules or impose an alternative decomposition of functionality of modules. For example, the modules discussed herein may be decomposed into submodules to be executed as multiple computer processes, and, optionally, on multiple computers. Moreover, alternative embodiments may combine multiple instances of a particular module or submodule. Furthermore, the operations may be combined or the functionality of the operations may be distributed in additional operations in accordance with the invention. Alternatively, such actions may be embodied in the structure of circuitry that implements such functionality, such as the micro-code of a complex instruction set computer (CISC), firmware programmed into programmable or erasable/programmable devices, the configuration of a field-programmable gate array (FPGA), the design of a gate array or full-custom application-specific integrated circuit (ASIC), or the like.

Each of the blocks of the flow diagrams of the process 100 performed by the antigen prediction apparatus 410 may be executed by a module (of software modules 522) or a portion of a module. The processes may be embodied in a non-transient machine-readable and/or computer-readable medium for configuring a computer system to execute the method. The software modules may be stored within and/or transmitted to a computer system memory to configure the computer system to perform the functions of the module.

For example, as shown in FIG. 9, the modules 522 may include:
a differential splicing module 412 that implements one or more differential splicing algorithms to identify a set of splice variants that are differentially spliced between the test samples and the reference samples (e.g., MISO 412a and/or rMATS 412b);
a sequence identification module 414 that determines, for each splice variant in the set, non-overlapping sequence between the splice variant and a corresponding second splice variant of the same gene; and
a peptide binding module 416 that predicts (e.g., using NetMHCPan or another similar prediction method), from one or more amino acid translations of the non-overlapping sequence, one or more candidate HLA-binding peptides.

The computing device 410 normally processes information according to a program (a list of internally stored instructions such as a particular application program and/or an operating system) and produces resultant output information via input/output (I/O) devices 530. A computer process typically includes an executing (running) program or portion of a program, current program values and state information, and the resources used by the operating system to manage the execution of the process. A parent process may spawn other, child processes to help perform the overall functionality of the parent process.

Because the parent process specifically spawns the child processes to perform a portion of the overall functionality of the parent process, the functions performed by child processes (and grandchild processes, etc.) may sometimes be described as being performed by the parent process.

Methods of Characterising and/or Treating

Disclosed herein is a method of characterising a medical condition in a subject, the method comprising determining the level (or presence) of one or more shared antigens identified according to a method as defined herein, wherein an increased level of the one or more shared antigens as compared to a reference characterises the medical condition as one that is associated with the expression of the one or more shared antigens.

The term “an increased level of the one or more shared antigens as compared to a reference” may refer to an increase of at least 5%, at least 10%, at least 20%, at least 30%, at least 40%, at least 50%, at least 60%, at least 70%, at least 80%, at 90%, at least 100% or at least 200% or more of the one or more shared antigens as compared to a reference.

In some embodiments, the one or more shared antigens are bound to HLA molecules and presented on the surface of one or more cells. The subject may be further treated with a suitable immunotherapy that recognises the one or more shared antigens.

Disclosed herein is a method of characterising a medical condition in a subject, the method comprising determining the level (or presence) of one or more shared antigens identified according to a method as defined herein, wherein an increased level of the one or more shared antigens as compared to a reference (or a presence of the one or more shared antigens) characterises the medical condition as one that is likely to be responsive to treatment with a suitable immunotherapy.

Suitable immunotherapies include, for example, autologous cell therapies; T-cell receptor-based therapies; antibody-based therapies and immunomodulatory compounds such as, for example, vaccines.

In some embodiments, the medical condition is cancer.

Disclosed herein is a method of treating a medical condition in a subject, the method comprising (a) determining the level (or presence) of one or more shared antigens identified according to a method as defined herein, wherein an increased level of the one or more shared antigens as compared to a reference (or a presence of the one or more shared antigens) characterises the medical condition as one that is associated with the expression of the one or more shared antigens, and (b) treating the subject found to have a medical condition that is associated with the expression of the one or more shared antigens.

Disclosed herein is a method of treating a medical condition in a subject, the method comprising (a) determining the level of one or more shared antigens identified according to a method as defined herein, wherein an increased level of the one or more shared antigens as compared to a reference (or a presence of the one or more shared antigens) characterises the medical condition as one that is likely to be responsive to treatment with a suitable immunotherapy, and (b) treating the subject found to have a medical condition that is likely to be responsive to treatment with a suitable immunotherapy.

The method of detecting a shared antigen (such as a HLA binding peptide or splice variant) in a sample may involve the use of PCR to detect a splice variant that encodes the shared antigen. PCR is performed using compositions derived from patient samples and a pair of primers that binds specifically to a splice variant nucleic acid. Detection of the shared antigen may be based on determining the size of the PCR product. Alternatively, detection may be based on detecting the binding of a labelled probe to a specific splice isoform during PCR; for example TaqMan real-time PCR.

In another approach, a shared antigen may be detected using hybridization of probes that are selective for the splice isoform. Compositions derived from patient samples may be used for the hybridization of probes that are selective for the splice isoform. The probe may bind to sequences that are present in the splice site junction or to other sequences that are present in the splice isoform (for example, inclusion of introns [IRE], exons [SIE,MXE] or parts of exons [A5E, A3E]).

In yet another approach, a shared antigen may be detected using antibodies. Compositions derived from patient samples can be contacted with antibodies and immunohistochemistry and western blots may be used to detect shared antigens.

Alternatively, RNA-Seq data may be used to detect a shared antigen.

The above methods are all well known to those of ordinary skill in the art.

Also disclosed herein is a method of characterising a cancer in a subject, the method comprising determining the level of a MAP/microtubule affinity-regulating kinase 3 (MARK3) splice variant in a sample obtained from the subject, wherein an increased level of MARK3 splice variant as compared to a reference characterises the cancer as one that is associated with the expression of the MARK3 splice variant.

Also disclosed herein is a method of characterising a cancer in a subject, the method comprising determining the level (or presence) of a MAP/microtubule affinity-regulating kinase 3 (MARK3) splice variant in a sample obtained from the subject, wherein an increased level of MARK3 splice variant as compared to a reference (or a presence of MARK3 splice variant) characterises the cancer as one that is likely to be responsive to treatment with a suitable immunotherapy.

Also disclosed herein is a method of treating cancer in a subject, the method comprising (a) determining the level (or presence) of a MARK3 splice variant in a sample obtained from the subject, wherein an increased level of MARK3 splice variant as compared to a reference (or a presence of MARK3 splice variant) characterises the cancer as one that is associated with the expression of the MARK3 splice variant, and (b) treating the subject found to have a cancer that is associated with the expression of the MARK3 splice variant.

Also disclosed herein is a method of treating cancer in a subject, the method comprising (a) determining the level (or a presence) of a MARK3 splice variant in a sample obtained from the subject, wherein an increased level of MARK3 splice variant as compared to a reference (or a presence of MARK3 splice variant) characterises the cancer as one that is likely to be responsive to treatment with a suitable immunotherapy, and (b) treating the subject found likely to be responsive to treatment with a suitable immunotherapy.

Also disclosed herein is a method of characterising a cancer in a subject, the method comprising determining the level of a NBPF9 splice variant in a sample obtained from the subject, wherein an increased level of NBPF9 splice variant as compared to a reference characterises the cancer as one that is associated with the expression of the NBPF9 splice variant.

Also disclosed herein is a method of characterising a cancer in a subject, the method comprising determining the level (or presence) of a NBPF9 splice variant in a sample obtained from the subject, wherein an increased level of NBPF9 splice variant as compared to a reference (or a presence of NBPF9 splice variant) characterises the cancer as one that is likely to be responsive to treatment with a suitable immunotherapy.

Also disclosed herein is a method of treating cancer in a subject, the method comprising (a) determining the level (or presence) of a NBPF9 splice variant in a sample obtained from the subject, wherein an increased level of NBPF9 splice variant as compared to a reference (or a presence of NBPF9 splice variant) characterises the cancer as one that is associated with the expression of the NBPF9 splice variant, and (b) treating the subject found to have a cancer that is associated with the expression of the NBPF9 splice variant.

Also disclosed herein is a method of treating cancer in a subject, the method comprising (a) determining the level (or a presence) of a NBPF9 splice variant in a sample obtained from the subject, wherein an increased level of NBPF9 splice variant as compared to a reference (or a presence of NBPF9 splice variant) characterises the cancer as one that is likely to be responsive to treatment with a suitable immunotherapy, and (b) treating the subject found likely to be responsive to treatment with a suitable immunotherapy.

Also disclosed herein is a method of characterising a cancer in a subject, the method comprising determining the level of a PARD3 splice variant in a sample obtained from the subject, wherein an increased level of PARD3 splice variant as compared to a reference characterises the cancer as one that is associated with the expression of the PARD3 splice variant.

Also disclosed herein is a method of characterising a cancer in a subject, the method comprising determining the level (or presence) of a PARD3 splice variant in a sample obtained from the subject, wherein an increased level of PARD3 splice variant as compared to a reference (or a presence of PARD3 splice variant) characterises the cancer as one that is likely to be responsive to treatment with a suitable immunotherapy.

Also disclosed herein is a method of treating cancer in a subject, the method comprising (a) determining the level (or presence) of a PARD3 splice variant in a sample obtained from the subject, wherein an increased level of PARD3 splice variant as compared to a reference (or a presence of PARD3 splice variant) characterises the cancer as one that is associated with the expression of the PARD3 splice variant, and (b) treating the subject found to have a cancer that is associated with the expression of the PARD3 splice variant.

Also disclosed herein is a method of treating cancer in a subject, the method comprising (a) determining the level (or a presence) of a PARD3 splice variant in a sample obtained from the subject, wherein an increased level of PARD3 splice variant as compared to a reference (or a presence of PARD3 splice variant) characterises the cancer as one that is likely to be responsive to treatment with a suitable immunotherapy, and (b) treating the subject found likely to be responsive to treatment with a suitable immunotherapy.

Also disclosed herein is a method of characterising a cancer in a subject, the method comprising determining the level of a ZC3HAV1 splice variant in a sample obtained from the subject, wherein an increased level of ZC3HAV1 splice variant as compared to a reference characterises the cancer as one that is associated with the expression of the ZC3HAV1 splice variant.

Also disclosed herein is a method of characterising a cancer in a subject, the method comprising determining the level (or presence) of a ZC3HAV1 splice variant in a sample obtained from the subject, wherein an increased level of ZC3HAV1 splice variant as compared to a reference (or a presence of ZC3HAV1 splice variant) characterises the cancer as one that is likely to be responsive to treatment with a suitable immunotherapy.

Also disclosed herein is a method of treating cancer in a subject, the method comprising (a) determining the level (or presence) of a ZC3HAV1 splice variant in a sample obtained from the subject, wherein an increased level of ZC3HAV1 splice variant as compared to a reference (or a presence of ZC3HAV1 splice variant) characterises the cancer as one that is associated with the expression of the ZC3HAV1 splice variant, and (b) treating the subject found to have a cancer that is associated with the expression of the ZC3HAV1 splice variant.

Also disclosed herein is a method of treating cancer in a subject, the method comprising (a) determining the level (or a presence) of a ZC3HAV1 splice variant in a sample obtained from the subject, wherein an increased level of ZC3HAV1 splice variant as compared to a reference (or a presence of ZC3HAV1 splice variant) characterises the cancer as one that is likely to be responsive to treatment with a suitable immunotherapy, and (b) treating the subject found likely to be responsive to treatment with a suitable immunotherapy.

Also disclosed herein is a method of characterising a cancer in a subject, the method comprising determining the level of a YAF2 splice variant in a sample obtained from the subject, wherein an increased level of YAF2 splice variant as compared to a reference characterises the cancer as one that is associated with the expression of the YAF2 splice variant.

Also disclosed herein is a method of characterising a cancer in a subject, the method comprising determining the level (or presence) of a YAF2 splice variant in a sample obtained from the subject, wherein an increased level of YAF2 splice variant as compared to a reference (or a presence of YAF2 splice variant) characterises the cancer as one that is likely to be responsive to treatment with a suitable immunotherapy.

Also disclosed herein is a method of treating cancer in a subject, the method comprising (a) determining the level (or presence) of a YAF2 splice variant in a sample obtained from the subject, wherein an increased level of YAF2 splice variant as compared to a reference (or a presence of YAF2 splice variant) characterises the cancer as one that is associated with the expression of the YAF2 splice variant, and (b) treating the subject found to have a cancer that is associated with the expression of the YAF2 splice variant.

Also disclosed herein is a method of treating cancer in a subject, the method comprising (a) determining the level (or a presence) of a YAF2 splice variant in a sample obtained from the subject, wherein an increased level of YAF2 splice variant as compared to a reference (or a presence of YAF2 splice variant) characterises the cancer as one that is likely to be responsive to treatment with a suitable immunotherapy, and (b) treating the subject found likely to be responsive to treatment with a suitable immunotherapy.

Also disclosed herein is a method of characterising a cancer in a subject, the method comprising determining the level of a CAMKK1 splice variant in a sample obtained from the subject, wherein an increased level of CAMKK1 splice variant as compared to a reference characterises the cancer as one that is associated with the expression of the CAMKK1 splice variant.

Also disclosed herein is a method of characterising a cancer in a subject, the method comprising determining the level (or presence) of a CAMKK1 splice variant in a sample obtained from the subject, wherein an increased level of CAMKK1 splice variant as compared to a reference (or a presence of CAMKK1 splice variant) characterises the cancer as one that is likely to be responsive to treatment with a suitable immunotherapy.

Also disclosed herein is a method of treating cancer in a subject, the method comprising (a) determining the level (or presence) of a CAMKK1 splice variant in a sample obtained from the subject, wherein an increased level of CAMKK1 splice variant as compared to a reference (or a presence of CAMKK1 splice variant) characterises the cancer as one that is associated with the expression of the CAMKK1 splice variant, and (b) treating the subject found to have a cancer that is associated with the expression of the CAMKK1 splice variant.

Also disclosed herein is a method of treating cancer in a subject, the method comprising (a) determining the level (or a presence) of a CAMKK1 splice variant in a sample obtained from the subject, wherein an increased level of CAMKK1 splice variant as compared to a reference (or a presence of CAMKK1 splice variant) characterises the cancer as one that is likely to be responsive to treatment with a suitable immunotherapy, and (b) treating the subject found likely to be responsive to treatment with a suitable immunotherapy.

Also disclosed herein is a method of characterising a cancer in a subject, the method comprising determining the level of a LRR1 splice variant in a sample obtained from the subject, wherein an increased level of LRR1 splice variant as compared to a reference characterises the cancer as one that is associated with the expression of the LRR1 splice variant.

Also disclosed herein is a method of characterising a cancer in a subject, the method comprising determining the level (or presence) of a LRR1 splice variant in a sample obtained from the subject, wherein an increased level of LRR1 splice variant as compared to a reference (or a presence of LRR1 splice variant) characterises the cancer as one that is likely to be responsive to treatment with a suitable immunotherapy.

Also disclosed herein is a method of treating cancer in a subject, the method comprising (a) determining the level (or presence) of a LRR1 splice variant in a sample obtained from the subject, wherein an increased level of LRR1 splice variant as compared to a reference (or a presence of LRR1 splice variant) characterises the cancer as one that is associated with the expression of the LRR1 splice variant, and (b) treating the subject found to have a cancer that is associated with the expression of the LRR1 splice variant.

Also disclosed herein is a method of treating cancer in a subject, the method comprising (a) determining the level (or a presence) of a LRR1 splice variant in a sample obtained from the subject, wherein an increased level of LRR1 splice variant as compared to a reference (or a presence of LRR1 splice variant) characterises the cancer as one that is likely to be responsive to treatment with a suitable immunotherapy, and (b) treating the subject found likely to be responsive to treatment with a suitable immunotherapy.

Also disclosed herein is a method of characterising a cancer in a subject, the method comprising determining the level of a ZNF670 splice variant in a sample obtained from the subject, wherein an increased level of ZNF670 splice variant as compared to a reference characterises the cancer as one that is associated with the expression of the ZNF670 splice variant.

Also disclosed herein is a method of characterising a cancer in a subject, the method comprising determining the level (or presence) of a ZNF670 splice variant in a sample obtained from the subject, wherein an increased level of ZNF670 splice variant as compared to a reference (or a presence of ZNF670 splice variant) characterises the cancer as one that is likely to be responsive to treatment with a suitable immunotherapy.

Also disclosed herein is a method of treating cancer in a subject, the method comprising (a) determining the level (or presence) of a ZNF670 splice variant in a sample obtained from the subject, wherein an increased level of ZNF670 splice variant as compared to a reference (or a presence of ZNF670 splice variant) characterises the cancer as one that is associated with the expression of the ZNF670 splice variant, and (b) treating the subject found to have a cancer that is associated with the expression of the ZNF670 splice variant.

Also disclosed herein is a method of treating cancer in a subject, the method comprising (a) determining the level (or a presence) of a ZNF670 splice variant in a sample obtained from the subject, wherein an increased level of ZNF670 splice variant as compared to a reference (or a presence of ZNF670 splice variant) characterises the cancer as one that is likely to be responsive to treatment with a suitable immunotherapy, and (b) treating the subject found likely to be responsive to treatment with a suitable immunotherapy.

Also disclosed herein is a method of characterising a cancer in a subject, the method comprising determining the level of a GRINA splice variant in a sample obtained from the subject, wherein an increased level of GRINA splice variant as compared to a reference characterises the cancer as one that is associated with the expression of the GRINA splice variant.

Also disclosed herein is a method of characterising a cancer in a subject, the method comprising determining the level (or presence) of a GRINA splice variant in a sample obtained from the subject, wherein an increased level of GRINA splice variant as compared to a reference (or a presence of GRINA splice variant) characterises the cancer as one that is likely to be responsive to treatment with a suitable immunotherapy.

Also disclosed herein is a method of treating cancer in a subject, the method comprising (a) determining the level (or presence) of a GRINA splice variant in a sample obtained from the subject, wherein an increased level of GRINA splice variant as compared to a reference (or a presence of GRINA splice variant) characterises the cancer as one that is associated with the expression of the GRINA splice variant, and (b) treating the subject found to have a cancer that is associated with the expression of the GRINA splice variant.

Also disclosed herein is a method of treating cancer in a subject, the method comprising (a) determining the level (or a presence) of a GRINA splice variant in a sample obtained from the subject, wherein an increased level of GRINA splice variant as compared to a reference (or a presence of GRINA splice variant) characterises the cancer as one that is likely to be responsive to treatment with a suitable immunotherapy, and (b) treating the subject found likely to be responsive to treatment with a suitable immunotherapy.

Also disclosed herein is a method of characterising a cancer in a subject, the method comprising determining the level of a MZF1 splice variant in a sample obtained from the subject, wherein an increased level of MZF1 splice variant as compared to a reference characterises the cancer as one that is associated with the expression of the MZF1 splice variant.

Also disclosed herein is a method of characterising a cancer in a subject, the method comprising determining the level (or presence) of a MZF1 splice variant in a sample obtained from the subject, wherein an increased level of MZF1 splice variant as compared to a reference (or a presence of MZF1 splice variant) characterises the cancer as one that is likely to be responsive to treatment with a suitable immunotherapy.

Also disclosed herein is a method of treating cancer in a subject, the method comprising (a) determining the level (or presence) of a MZF1 splice variant in a sample obtained from the subject, wherein an increased level of MZF1 splice variant as compared to a reference (or a presence of MZF1 splice variant) characterises the cancer as one that is associated with the expression of the MZF1 splice variant, and (b) treating the subject found to have a cancer that is associated with the expression of the MZF1 splice variant.

Also disclosed herein is a method of treating cancer in a subject, the method comprising (a) determining the level (or a presence) of a MZF1 splice variant in a sample obtained from the subject, wherein an increased level of MZF1 splice variant as compared to a reference (or a presence of MZF1 splice variant) characterises the cancer as one that is likely to be responsive to treatment with a suitable immunotherapy, and (b) treating the subject found likely to be responsive to treatment with a suitable immunotherapy.

Compositions

Compositions may comprise sample cDNA having a cDNA expression profile characteristic of a cancer patient and at least one primer or probe that binds specifically to the cDNA molecule, wherein the sample cDNA comprises a cDNA molecule corresponding to a shared antigen. In some embodiments, the sample cDNA is a tissue or saliva sample cDNA. The primer or probe may be attached to a label. The composition may further comprise a DNA polymerase.

In some embodiments, the shared antigen is a MARK3, NBPF9, PARD3, ZC3HAV1, YAF2, CAMKK1, LRR1, ZNF670, GRINA or MZF1 splice variant.

The composition may also comprise sample RNA having a RNA expression profile characteristic of a cancer patient. The composition may comprise at least one primer (e.g. an oligo-dT primer) or probe that binds to the RNA molecules. The composition may further comprise a reverse transcriptase for generating cDNA molecules.

The composition may also comprise tissue sections or protein lysates that have been extracted from patient biopsy samples, wherein the composition can be reacted with probes or antibodies to identify the presence of a shared antigen.

PCR

The term “Polymerase chain reaction” or “PCR” means a reaction for the in vitro amplification of specific nucleic acid sequences by the simultaneous primer extension of complementary strands of nucleic acid molecules. In other words, PCR is a reaction for making multiple copies or replicates of a target nucleic acid flanked by primer sites, such reaction comprising one or more repetitions of the following steps: (i) denaturing the target nucleic acid, (ii) annealing primers to the primer sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art. The term “PCR” encompasses derivative forms of the reaction, including but not limited to, Reverse transcription-PCR, real-time PCR, nested PCR, quantitative PCR, multiplexed PCR, and the like.

Primers

The term “primer” as used herein refers to a polymer of nucleotides capable of acting as a point of initiation of DNA synthesis when annealed to a nucleic acid template under conditions in which synthesis of a primer extension product is initiated, i.e., in the presence of four different nucleotide triphosphates and a polymerase in an appropriate buffer (“buffer” includes pH, ionic strength, cofactors, etc.) and at a suitable temperature.

The primers used in the amplification steps of the invention may be fully complementary or substantially complementary to the target sequences.

Generally, a primer will be between 12 and 100 nucleotides, more preferably between 10 and 80 nucleotides; more preferably between 15 and 30 nucleotides; and more preferably between 15 and 25 nucleotides.

The method as disclosed herein may comprise detecting a shared antigen with a pair of primers that binds specifically to a shared antigen nucleic acid. One or more primers may be labelled by coupling to a detectable substance, such as a fluorophore.

The term “labelled”, with regard to, for example, a primer, antibody or probe, is intended to encompass direct labelling of the probe by coupling (i.e., physically linking) a detectable substance to the probe, as well as indirect labelling of the probe by reactivity with another reagent that is directly labelled. Examples of indirect labelling include detection of a primary antibody using a fluorescently labelled secondary antibody and end-labelling of a DNA probe with biotin such that it can be detected with fluorescently labelled streptavidin.

Probes

The term “probe” refers to any molecule which is capable of selectively binding to a specifically intended target molecule, for example, a nucleotide transcript or polypeptide. Probes can be either synthesized by one skilled in the art, or derived from appropriate biological preparations. For purposes of detection of the target molecule, probes may be specifically designed to be labelled. Examples of molecules that can be utilized as probes include, but are not limited to, RNA, DNA, proteins, antibodies, and organic molecules. In some embodiments, a probe can be surface immobilized. Where nucleic acids (such as oligonucleotides) are used they may be capable of binding in a base-specific manner to another strand of nucleic acid. Hybridization may occur between complementary nucleic acid strands or between nucleic acid strands that contain minor regions of mismatch. Such probes include peptide nucleic acids, and other nucleic acid analogs and nucleic acid mimetics that are known in the art.

The term “hybridization” as used herein, refers to the formation of a duplex structure by two single stranded nucleic acids due to complementary base pairing. Hybridization can occur between complementary nucleic acid strands or between nucleic acid strands that contain minor regions of mismatch. The melting temperature, or “Tm” measures stability of a nucleic acid duplex. The Tm is the temperature (under defined ionic strength and pH) at which 50% of the base pairs have dissociated. Those skilled in the art of nucleic acid technology can determine duplex stability empirically considering a number of variables including, for example, the length of the nucleic acids, base composition and sequence, ionic strength, and incidence of mismatched base pairs.

Antibodies

The method as disclosed herein may comprise detecting a shared antigen (e.g. splice variant and/or a splice variant antigen) with an antibody that binds specifically to the shared antigen. The antibody may be labelled by coupling to a detectable substance such as a fluorophore or an enzyme.

Kits

The disclosure may provide for development and use of kits comprising reagents (such as antibodies, probes or primers) for detecting or measuring the level of a shared antigen (e.g. splice variant and/or a splice variant antigen), as defined herein, in a sample. The kits may also comprise assay reagents and suitable buffer.

Treatment

The method may comprise administering an anti-cancer therapy or agent to a subject found to have cancer that expresses the one or more shared antigens. The anti-cancer therapy or agent may include chemotherapy, radiation therapy, a targeted therapy, immunotherapy, or a combination thereof. In some embodiments, the method may comprise detecting the presence of cancer antigen or target to identify which patients might be suitable candidates for administering the anti-cancer therapy or agent.

Determining the Presence of Antigen-Specific T Lymphocytes

Disclosed herein is a method of characterising a medical condition in a subject, the method comprising determining the level (or presence) of T lymphocytes that bind specifically to one or more shared antigens identified according to a method as defined herein, wherein an increased level of the T lymphocytes as compared to a reference (or a presence of T lymphocytes that bind specifically to one or more shared antigens) characterises the medical condition as one that is associated with the expression of one or more shared antigens

The method may comprise determining (a) the level (or presence) of T lymphocytes that bind specifically to one or more shared antigens identified according to a method as defined herein and (b) the level (or presence) of the one or more shared antigen in a sample obtained from the subject. In some embodiments, the method may comprise solely determining the level (or presence) of the T lymphocytes that bind specifically to one or more shared antigens identified according to a method as defined herein. In another embodiment, the method may comprise determining (a) the level (or presence) of T lymphocytes that bind specifically to one or more shared antigens identified according to a method as defined herein and (b) the phenotype of such antigen-specific T lymphocytes.

Disclosed herein is a method of treating a medical condition in a subject, the method comprising a) determining the level (or presence) of T lymphocytes that bind specifically to one or more shared antigens identified according to a method as defined herein, wherein an increased level of the T lymphocytes as compared to a reference (or a presence of T lymphocytes that bind specifically to one or more shared antigens) characterises the medical condition as one that is associated with the expression of one or more shared antigens; and b) treating the subject found to have a medical condition associated with the expression of the one or more shared antigens.

In some embodiments, there is provided a labelled biomolecule comprising a HLA molecule bound to a shared antigen for use in detecting the presence or determining the level of T lymphocytes that binds specifically to the shared antigen.

The term “T lymphocyte” (also known as T cell) may refer to a CD4⁺ T lymphocyte (such as an immature CD4⁺ T lymphocyte or a mature CD4⁺ helper T lymphocyte). The term “T lymphocyte” may also refer to a CD8⁺ T lymphocyte (such as an immature CD8⁺ T lymphocyte or a mature CD8⁺ cytotoxic T lymphocyte). The term “T lymphocyte” may also refer to a mixture of CD4⁺ T lymphocytes as well as CD8⁺ T lymphocytes.

In some embodiments, the T lymphocyte is a non-naïve T-lymphocyte. In some embodiments, the T lymphocytes is a naïve T-lymphocyte. In some embodiments, the T lymphocytes might also refer to antigen experienced T lymphocytes.

In some embodiments, the T lymphocyte is a cytotoxic T lymphocyte. A cytotoxic T lymphocyte (also known as cytotoxic T cell, Tc, CTL, T-killer cell, cytolytic T cell, CD8+ T-cell or killer T cell) is a T lymphocyte that kills cancer cells, infected cells or cells that are damaged in other ways.

In some embodiments, the T lymphocyte is a helper T lymphocyte. A helper T lymphocyte is a T lymphocyte that help the activity of other immune cells by releasing T cell cytokines to regulate immune responses.

In some embodiments, the shared antigen or fragment thereof is presented on the surface of an antigen-presenting cell (e.g. a professional antigen-presenting cell or a cancer cell). The shared antigen or fragment thereof may be bound to a HLA molecule and presented on the surface of the antigen-presenting cell or the cancer cell.

The HLA as referred to herein may refer to a HLA from MHC class I or MHC class II.

In one embodiment, the HLA molecule is MHC class I molecule selected from the group consisting of HLA-A11, HLA-A02 and/or HLA-A24.

Disclosed herein is a method of characterising a cancer in a subject, the method comprising determining the level (or presence) of T lymphocytes that bind specifically to a MARK3 splice variant in a subject, wherein an increased level of the T lymphocytes as compared to a reference (or a presence of the T lymphocytes) characterises the cancer as one that is associated with the expression of the MARK3 splice variant.

Disclosed herein is a method of characterising a cancer in a subject, the method comprising determining the level (or presence) of T lymphocytes that bind specifically to a NBPF9 splice variant in a subject, wherein an increased level of the T lymphocytes as compared to a reference (or a presence of the T lymphocytes) characterises the cancer as one that is associated with the expression of the NBPF9 splice variant.

Disclosed herein is a method of characterising a cancer in a subject, the method comprising determining the level (or presence) of T lymphocytes that bind specifically to a PARD3 splice variant in a subject, wherein an increased level of the T lymphocytes as compared to a reference (or a presence of the T lymphocytes) characterises the cancer as one that is associated with the expression of the PARD3 splice variant.

Disclosed herein is a method of characterising a cancer in a subject, the method comprising determining the level (or presence) of T lymphocytes that bind specifically to a ZC3HAV1 splice variant in a subject, wherein an increased level of the T lymphocytes as compared to a reference (or a presence of the T lymphocytes) characterises the cancer as one that is associated with the expression of the ZC3HAV1 splice variant.

Disclosed herein is a method of characterising a cancer in a subject, the method comprising determining the level (or presence) of T lymphocytes that bind specifically to a YAF2 splice variant in a subject, wherein an increased level of the T lymphocytes as compared to a reference (or a presence of the T lymphocytes) characterises the cancer as one that is associated with the expression of the YAF2 splice variant.

Disclosed herein is a method of characterising a cancer in a subject, the method comprising determining the level (or presence) of T lymphocytes that bind specifically to a CAMKK1 splice variant in a subject, wherein an increased level of the T lymphocytes as compared to a reference (or a presence of the T lymphocytes) characterises the cancer as one that is associated with the expression of the CAMKK1 splice variant.

Disclosed herein is a method of characterising a cancer in a subject, the method comprising determining the level (or presence) of T lymphocytes that bind specifically to a LRR1 splice variant in a subject, wherein an increased level of the T lymphocytes as compared to a reference (or a presence of the T lymphocytes) characterises the cancer as one that is associated with the expression of the LRR1 splice variant.

Disclosed herein is a method of characterising a cancer in a subject, the method comprising determining the level (or presence) of T lymphocytes that bind specifically to a ZNF670 splice variant in a subject, wherein an increased level of the T lymphocytes as compared to a reference (or a presence of the T lymphocytes) characterises the cancer as one that is associated with the expression of the ZNF670 splice variant.

Disclosed herein is a method of characterising a cancer in a subject, the method comprising determining the level (or presence) of T lymphocytes that bind specifically to a GRINA splice variant in a subject, wherein an increased level of the T lymphocytes as compared to a reference (or a presence of the T lymphocytes) characterises the cancer as one that is associated with the expression of the GRINA splice variant.

Disclosed herein is a method of characterising a cancer in a subject, the method comprising determining the level (or presence) of T lymphocytes that bind specifically to a MZF1 splice variant in a subject, wherein an increased level of the T lymphocytes as compared to a reference (or a presence of the T lymphocytes) characterises the cancer as one that is associated with the expression of the MZF1 splice variant.

Cell Therapy

Disclosed herein is a method of treating a medical condition in a subject, the method comprising: (a) determining the level (or presence) of T lymphocytes that binds specifically to one or more shared antigens identified according to a method as defined herein, wherein an increased level of the T lymphocytes as compared to a reference (or a presence of T lymphocytes) characterises the medical condition in the subject as one that is associated with the expression of one or more shared antigens; (b) isolating and expanding the population of T lymphocytes ex vivo; and (c) administering the expanded population of T lymphocytes to the subject to treat the medical condition found to be associated with the expression of the one or more shared antigens.

Disclosed herein is a method of treating a medical condition in a subject, the method comprising: (a) determining the level (or presence) of T lymphocytes that binds specifically to one or more shared antigens identified according to the method as defined herein, wherein an increased level of the T lymphocytes as compared to a reference (or a presence of T lymphocytes) indicates that the subject is likely to be responsive to treatment with a suitable immunotherapy; (b) isolating and expanding the population of T lymphocytes ex vivo; and (c) administering the expanded population of T lymphocytes to the subject to treat the medical condition in the subject.

Disclosed herein is a method of treating a medical condition in a subject, the method comprising: (a) isolating a population of T lymphocytes that binds specifically to one or more shared antigens identified according to a method as defined herein in a subject suffering from the medical condition, and expanding the population of T lymphocytes ex vivo; and (b) administering the expanded population of T lymphocytes to the subject to treat the medical condition in the subject. In some embodiments the medical condition is cancer.

The method may comprise obtaining a population of peripheral blood mononuclear cells (PBMCs). The PBMCs may be stimulated with a shared antigen to stimulate expansion of T lymphocytes that recognise the shared antigen. In some embodiments, the method may comprise obtaining PBMCs to isolate monocytes for differentiation into dendritic cells to stimulate the expansion of T lymphocytes that recognise the shared antigen. In some embodiments, the method may comprise obtaining PBMC for the generation of EBV-transformed B cells for the expansion of T lymphocytes that recognise the shared antigen. In some embodiments, combinations of dendritic cells and EBV-transformed B cells may be used for the expansion of T lymphocytes that recognise the shared antigen.

The term “administering” refers to contacting, applying or providing a suitable therapy to a subject suffering from a medical condition. The medical condition may be cancer and the suitable therapy may be any one of a number of anti-cancer immunotherapies.

The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

The terms “patient”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the phylum Chordata including primates (e.g., humans, monkeys and apes, and includes species of monkeys such from the genus Macaca (e.g., cynomologus monkeys such as Macaca fascicularis, and/or rhesus monkeys (Macaca mulatta) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. In some embodiments, the subject is human.

Disclosed herein is a method of characterising a cancer in a subject, the method comprising determining the level of T lymphocytes that bind specifically to a MARK3 splice variant in a subject, wherein an increased level of the T lymphocytes as compared to a reference characterises the cancer as one that is associated with the expression of the MARK3 splice variant.

Disclosed herein is a method of treating cancer in a subject, the method comprising:
(a) determining the level of T lymphocytes that binds specifically to a MARK3 splice variant in a subject, wherein an increased level of the T lymphocyte as compared to a reference characterises the cancer as one that is associated with the expression of the MARK3 splice variant;
(b) isolating and expanding the population of T lymphocytes ex vivo;
(c) administering the expanded population of T lymphocytes to the subject to treat the cancer in the subject.

Disclosed herein is a method of treating cancer in a subject, the method comprising:
(a) isolating a population of T lymphocytes that binds specifically to a MARK3 splice variant from a subject suffering from cancer, and expanding the population of T lymphocytes ex vivo; and
(b) administering the expanded population of T lymphocytes to the subject to treat the cancer in the subject.

Disclosed herein is a method of characterising a cancer in a subject, the method comprising determining the level of T lymphocytes that bind specifically to a NBPF9 splice variant in a subject, wherein an increased level of the T lymphocytes as compared to a reference characterises the cancer as one that is associated with the expression of the NBPF9 splice variant.

Disclosed herein is a method of treating cancer in a subject, the method comprising:
(a) determining the level of T lymphocytes that binds specifically to a NBPF9 splice variant in a subject, wherein an increased level of the T lymphocyte as compared to a reference characterises the cancer as one that is associated with the expression of the NBPF9 splice variant;
(b) isolating and expanding the population of T lymphocytes ex vivo;
(c) administering the expanded population of T lymphocytes to the subject to treat the cancer in the subject.

Disclosed herein is a method of treating cancer in a subject, the method comprising:
(a) isolating a population of T lymphocytes that binds specifically to a NBPF9 splice variant from a subject suffering from cancer, and expanding the population of T lymphocytes ex vivo; and
(b) administering the expanded population of T lymphocytes to the subject to treat the cancer in the subject.

Disclosed herein is a method of characterising a cancer in a subject, the method comprising determining the level of T lymphocytes that bind specifically to a PARD3 splice variant in a subject, wherein an increased level of the T lymphocytes as compared to a reference characterises the cancer as one that is associated with the expression of the PARD3 splice variant.

Disclosed herein is a method of treating cancer in a subject, the method comprising:
(a) determining the level of T lymphocytes that binds specifically to a PARD3 splice variant in a subject, wherein an increased level of the T lymphocyte as compared to a reference characterises the cancer as one that is associated with the expression of the PARD3 splice variant;
(b) isolating and expanding the population of T lymphocytes ex vivo;
(c) administering the expanded population of T lymphocytes to the subject to treat the cancer in the subject.

Disclosed herein is a method of treating cancer in a subject, the method comprising:
(a) isolating a population of T lymphocytes that binds specifically to a PARD3 splice variant from a subject suffering from cancer, and expanding the population of T lymphocytes ex vivo; and
(b) administering the expanded population of T lymphocytes to the subject to treat the cancer in the subject.

Disclosed herein is a method of characterising a cancer in a subject, the method comprising determining the level of T lymphocytes that bind specifically to a ZC3HAV1 splice variant in a subject, wherein an increased level of the T lymphocytes as compared to a reference characterises the cancer as one that is associated with the expression of the ZC3HAV1 splice variant.

Disclosed herein is a method of treating cancer in a subject, the method comprising:
(a) determining the level of T lymphocytes that binds specifically to a ZC3HAV1 splice variant in a subject, wherein an increased level of the T lymphocyte as compared to a reference characterises the cancer as one that is associated with the expression of the ZC3HAV1 splice variant;
(b) isolating and expanding the population of T lymphocytes ex vivo;
(c) administering the expanded population of T lymphocytes to the subject to treat the cancer in the subject.

Disclosed herein is a method of treating cancer in a subject, the method comprising:
(a) isolating a population of T lymphocytes that binds specifically to a ZC3HAV1 splice variant from a subject suffering from cancer, and expanding the population of T lymphocytes ex vivo; and
(b) administering the expanded population of T lymphocytes to the subject to treat the cancer in the subject.

Disclosed herein is a method of characterising a cancer in a subject, the method comprising determining the level of T lymphocytes that bind specifically to a YAF2 splice variant in a subject, wherein an increased level of the T lymphocytes as compared to a reference characterises the cancer as one that is associated with the expression of the YAF2 splice variant.

Disclosed herein is a method of treating cancer in a subject, the method comprising:
(a) determining the level of T lymphocytes that binds specifically to a YAF2 splice variant in a subject, wherein an increased level of the T lymphocyte as compared to a reference characterises the cancer as one that is associated with the expression of the YAF2 splice variant;
(b) isolating and expanding the population of T lymphocytes ex vivo;
(c) administering the expanded population of T lymphocytes to the subject to treat the cancer in the subject.

Disclosed herein is a method of treating cancer in a subject, the method comprising:
(a) isolating a population of T lymphocytes that binds specifically to a YAF2 splice variant from a subject suffering from cancer, and expanding the population of T lymphocytes ex vivo; and
(b) administering the expanded population of T lymphocytes to the subject to treat the cancer in the subject.

Disclosed herein is a method of characterising a cancer in a subject, the method comprising determining the level of T lymphocytes that bind specifically to a CAMKK1 splice variant in a subject, wherein an increased level of the T lymphocytes as compared to a reference characterises the cancer as one that is associated with the expression of the CAMKK1 splice variant.

Disclosed herein is a method of treating cancer in a subject, the method comprising:
(a) determining the level of T lymphocytes that binds specifically to a CAMKK1 splice variant in a subject, wherein an increased level of the T lymphocyte as compared to a reference characterises the cancer as one that is associated with the expression of the CAMKK1 splice variant;
(b) isolating and expanding the population of T lymphocytes ex vivo; (c) administering the expanded population of T lymphocytes to the subject to treat the cancer in the subject.

Disclosed herein is a method of treating cancer in a subject, the method comprising:
(a) isolating a population of T lymphocytes that binds specifically to a CAMKK1 splice variant from a subject suffering from cancer, and expanding the population of T lymphocytes ex vivo; and
(b) administering the expanded population of T lymphocytes to the subject to treat the cancer in the subject.

Disclosed herein is a method of characterising a cancer in a subject, the method comprising determining the level of T lymphocytes that bind specifically to a LRR1 splice variant in a subject, wherein an increased level of the T lymphocytes as compared to a reference characterises the cancer as one that is associated with the expression of the LRR1 splice variant.

Disclosed herein is a method of treating cancer in a subject, the method comprising:
(a) determining the level of T lymphocytes that binds specifically to a LRR1 splice variant in a subject, wherein an increased level of the T lymphocyte as compared to a reference characterises the cancer as one that is associated with the expression of the LRR1 splice variant;
(b) isolating and expanding the population of T lymphocytes ex vivo;
(c) administering the expanded population of T lymphocytes to the subject to treat the cancer in the subject.

Disclosed herein is a method of treating cancer in a subject, the method comprising:
(a) isolating a population of T lymphocytes that binds specifically to a LRR1 splice variant from a subject suffering from cancer, and expanding the population of T lymphocytes ex vivo; and
(b) administering the expanded population of T lymphocytes to the subject to treat the cancer in the subject.

Disclosed herein is a method of characterising a cancer in a subject, the method comprising determining the level of T lymphocytes that bind specifically to a ZNF670 splice variant in a subject, wherein an increased level of the T lymphocytes as compared to a reference characterises the cancer as one that is associated with the expression of the ZNF670 splice variant.

Disclosed herein is a method of treating cancer in a subject, the method comprising:
(a) determining the level of T lymphocytes that binds specifically to a ZNF670 splice variant in a subject, wherein an increased level of the T lymphocyte as compared to a reference characterises the cancer as one that is associated with the expression of the ZNF670 splice variant;
(b) isolating and expanding the population of T lymphocytes ex vivo;
(c) administering the expanded population of T lymphocytes to the subject to treat the cancer in the subject.

Disclosed herein is a method of treating cancer in a subject, the method comprising:
(a) isolating a population of T lymphocytes that binds specifically to a ZNF670 splice variant from a subject suffering from cancer, and expanding the population of T lymphocytes ex vivo; and
(b) administering the expanded population of T lymphocytes to the subject to treat the cancer in the subject.

Disclosed herein is a method of characterising a cancer in a subject, the method comprising determining the level of T lymphocytes that bind specifically to a GRINA splice variant in a subject, wherein an increased level of the T lymphocytes as compared to a reference characterises the cancer as one that is associated with the expression of the GRINA splice variant.

Disclosed herein is a method of treating cancer in a subject, the method comprising:
(a) determining the level of T lymphocytes that binds specifically to a GRINA splice variant in a subject, wherein an increased level of the T lymphocyte as compared to a reference characterises the cancer as one that is associated with the expression of the GRINA splice variant;
(b) isolating and expanding the population of T lymphocytes ex vivo;
(c) administering the expanded population of T lymphocytes to the subject to treat the cancer in the subject.

Disclosed herein is a method of treating cancer in a subject, the method comprising:
(a) isolating a population of T lymphocytes that binds specifically to a GRINA splice variant from a subject suffering from cancer, and expanding the population of T lymphocytes ex vivo; and
(b) administering the expanded population of T lymphocytes to the subject to treat the cancer in the subject.

Disclosed herein is a method of characterising a cancer in a subject, the method comprising determining the level of T lymphocytes that bind specifically to a MZF1 splice variant in a subject, wherein an increased level of the T lymphocytes as compared to a reference characterises the cancer as one that is associated with the expression of the MZF1 splice variant.

Disclosed herein is a method of treating cancer in a subject, the method comprising:
(a) determining the level of T lymphocytes that binds specifically to a MZF1 splice variant in a subject, wherein an increased level of the T lymphocyte as compared to a reference characterises the cancer as one that is associated with the expression of the MZF1 splice variant;
(b) isolating and expanding the population of T lymphocytes ex vivo;
(c) administering the expanded population of T lymphocytes to the subject to treat the cancer in the subject.

Disclosed herein is a method of treating cancer in a subject, the method comprising:
(a) isolating a population of T lymphocytes that binds specifically to a MZF1 splice variant from a subject suffering from cancer, and expanding the population of T lymphocytes ex vivo; and
(b) administering the expanded population of T lymphocytes to the subject to treat the cancer in the subject.

TCR Sequences to Engineer Immune Cells for Treatment

By “TCR” is meant a molecule that has binding affinity for antigen protein or fragment thereof bound to a HLA molecule and which may be presented on the surface of the antigen-presenting cell or the target cell. It will be understood that this term extends to heterodimers of TRA and TRB chains or heterodimers of TRG and TRD chains.

TCRs are described using the International Immunogenetics (IMGT) TCR nomenclature, and links to the IMGT public database of TCR sequences. Native alpha-beta heterodimeric TCRs have an alpha chain and a beta chain. Broadly, each chain comprises variable, joining and constant regions, and the beta chain also usually contains a short diversity region between the variable and joining regions, but this diversity region is often considered as part of the joining region. Each variable region comprises three CDRs (Complementarity Determining Regions) embedded in a framework sequence, one being the hypervariable region named CDR3. There are several types of alpha chain variable (Vα) regions and several types of beta chain variable (V_{β}) regions distinguished by their framework, CDR1 and CDR2 sequences, and by a partly defined CDR3 sequence. The Vα types are referred to in IMGT nomenclature by a unique TRAV number. Thus “TRAV21” defines a TCR Vα region having unique framework and CDR1 and CDR2 sequences, and a CDR3 sequence which is partly defined by an amino acid sequence which is preserved from TCR to TCR but which also includes an amino acid sequence which varies from TCR to TCR. In the same way, “TRBV5-1” defines a TCR V_{β} region having unique framework and CDR1 and CDR2 sequences, but with only a partly defined CDR3 sequence.

The joining regions of the TCR are similarly defined by the unique IMGT TRAJ and TRBJ nomenclature, and the constant regions by the IMGT TRAC and TRBC nomenclature. The beta chain diversity region is referred to in IMGT nomenclature by the abbreviation TRBD, and, as mentioned, the concatenated TRBD/TRBJ regions are often considered together as the joining region. The α and β chains of αβ TCR's are generally regarded as each having two “domains”, namely variable and constant domains. The variable domain consists of a concatenation of variable region and joining region. In the present specification and claims, the term “TCR alpha variable domain” therefore refers to the concatenation of TRAV and TRAJ regions, and the term TCR alpha constant domain refers to the extracellular TRAC region, or to a C-terminal truncated TRAC sequence. Likewise the term “TCR beta variable domain” refers to the concatenation of TRBV and TRBD/TRBJ regions, and the term TCR beta constant domain refers to the extracellular TRBC region, or to a C-terminal truncated TRBC sequence.

The unique sequences defined by the IMGT nomenclature are widely known and accessible to those working in the TCR field. For example, they can be found in the IMGT public database. The “T cell Receptor Factsbook”, (2001) LeFranc and LeFranc, Academic Press, ISBN 0-1 2-441 352-8 also discloses sequences defined by the IMGT nomenclature, but because of its publication date and consequent time-lag, the information therein sometimes needs to be confirmed by reference to the IMGT database.

As will be obvious to those skilled in the art the mutation(s) in the TCRα chain sequence and/or TCR β chain sequence may be one or more of substitution(s), deletion(s) or insertion(s). These mutations can be carried out using any appropriate method including, but not limited to, those based on polymerase chain reaction (PCR), restriction enzyme based cloning, or ligation independent cloning (LIC) procedures.

The TCRs of the invention may be αβ heterodimers or may be in single chain format. Single chain formats include αβ TCR polypeptides of the V_{α}-L-V_{β}, V_{β}-L-V_{α}. V_{α}-C_{α}-L-V_{β} or V_{α}-L-V_{β}-C_{β} types, wherein V_{α} and V_{β} are TCR α and β variable regions respectively. Cα and C_{β} are TCR a and β constant regions respectively, and L is a linker sequence. For use as a targeting agent for delivering therapeutic agents to the antigen-presenting cell the TCR may be in soluble form (i.e. having no transmembrane or cytoplasmic domains). For stability, soluble αβ heterodimeric TCRs preferably have an introduced disulphide bond between residues of the respective constant domains, as described, for example, in WO 03/020763. One or both of the constant domains present in an αβ heterodimer of the invention may be truncated at the C terminus or C termini, for example by up to 15, or up to 10 or up to 8 or fewer amino acids. For use in adoptive therapy, an αβ heterodimeric TCR may, for example, be transfected as full length chains having both cytoplasmic and transmembrane domains. TCRs for use in adoptive therapy may contain a disulphide bond corresponding to that found in nature between the respective alpha and beta constant domains, additionally or alternatively a non-native disulphide bond may be present.

As will be obvious to those skilled in the art, it may be possible to truncate the sequences provided at the C-terminus and/or N-terminus thereof, by 1, 2, 3, 4, 5 or more residues, without substantially affecting the binding characteristics of the TCR. All such trivial variants are encompassed by the present invention.

Alpha-beta heterodimeric TCRs of the invention usually comprise an alpha chain TRAC constant domain sequence and a beta chain TRBC1 or TRBC2 constant domain sequence.

The alpha and beta chain constant domain sequences may be modified by truncation or substitution to delete the native disulphide bond between Cys4 of exon 2 of TRAC and Cys2 of exon 2 of TRBC1 or TRBC2. The alpha and beta chain constant domain sequences may also be modified by substitution of cysteine residues for Thr 48 of TRAC and Ser 57 of TRBC1 or TRBC2. The said cysteines forming a disulphide bond between the alpha and beta constant domains of the TCR.

In some embodiments, there is provided a method of producing a TCR; the method may comprise: 1) identifying and isolating T lymphocytes from patient or donor which binds specifically to the shared antigen or fragment thereof that is bound to the HLA molecule; and or 2) further identifying the sequence of antigen-binding molecules expressed by these T lymphocytes.

In some embodiments, there is provided a method of producing a TCR; the method may comprise: 1) isolation of PBMCs from patients or matched healthy donors; 2) isolation of antigen-presenting cells and T lymphocytes; 3) stimulation of T lymphocytes with a shared antigen identified according to a method as defined herein; 4) identifying and isolating T lymphocytes that bind specifically to the shared antigen or fragment thereof that is bound to the HLA molecule; 5) further identifying the sequence of the TCR expressed by these T lymphocytes.

The method for identifying TCR sequences that bind specifically to one or more shared antigens identified herein, may comprise one or more of the following steps:
a) isolation of antigen-specific T lymphocytes;
b) separation of antigen-specific T lymphocytes into individual cells;
c) preparation of nucleic acid from antigen-specific T lymphocytes; and
d) sequencing to obtain TCR sequences that are antigen-specific.

Isolation of antigen-specific T lymphocytes may be done by: 1) contacting one or more labelled biomolecules with one or more samples containing peripheral blood from respective patients having the medical condition or donors, and 2) isolating, from the one or more samples, T lymphocytes that are bound to labelled biomolecules. In some embodiments, the labelled biomolecule may be a HLA multimer and binding would indicate antigen specificity. In some embodiments, the labelled biomolecule may be an antibody that indicates activation of T lymphocytes upon recognition of antigen. For example, when an EBV-specific T lymphocyte encounters an EBV-infected cell, it would be activated to induce surface expression of CD107 or secrete IFN-γ. In some embodiments, the labelled biomolecule may be combinations of single or multiple HLA multimers and antibodies. In some embodiments, there may involve expansion of these antigen specific cells to facilitate obtaining more material for subsequent steps. The isolated antigen-specific T lymphocytes may consist of a polyclonal population of T lymphocytes that express multiple TCRs (each T lymphocyte expressing different versions of TRA and TRB).

Separation of antigen-specific T lymphocytes into individual cells, so that the sequences of individual TCRs can be identified, may be accomplished by a number of methods well known to those skilled in the art. These methods include, for example, sorting the population of antigen-specific T lymphocytes into individual cells using a FACs sorter, using microfluidics; using droplet emulsions; or separation may include the addition of barcode sequences to facilitate identification of individual T lymphocytes clones and subsequent pooling of antigen-specific T lymphocytes.

Preparation of nucleic acid from the antigen-specific T lymphocytes for the isolation of TCR sequences can likewise be accomplished by a number of methods well known to those skilled in the art. Either RNA or DNA may be used as the starting nucleic acid material. The nucleic acid is amplified by PCR to get enough material for isolating TCR sequences. TCR sequences may also be amplified directly from the nucleic acid from antigen-specific T lymphocytes. Amplification of TCR sequences may include generating a gene expression profile of the antigen-specific T lymphocytes to allow prioritization or ranking of TCR sequences.

Sequencing may be carried out by any number of sequencing modalities including but not limited to, for example, Sanger sequencing or next-generation sequencing to obtain TCR sequences.

The sequencing of T-Cell receptors may be carried out following the identification of antigen-specific T lymphocytes in accordance with step 156 of method 150 (FIG. 1(b)).

Alternatively, TCR sequences may be identified through screening a library of yeast or bacteriophages expressing TCRs on their surface. This involves identifying which TCR sequence is able to bind to a shared antigen or fragment thereof that is bound to the HLA molecule.

Disclosed herein is a nucleic acid encoding a T-cell receptor, wherein the T-cell receptor encoded by the nucleic acid is capable of specifically binding to a shared antigen or fragment thereof, wherein the shared antigen or fragment thereof is bound to a HLA molecule.

Provided herein is also an isolated T-cell receptor encoded by the nucleic acid as defined herein. In some embodiments, there is provided a T-cell receptor (TCR) that specifically binds to a shared antigen, wherein the shared antigen is bound to HLA molecule. The shared antigen may be presented on the surface of an antigen-presenting cell or cancer cell.

Once antigen-specific TCRs against the shared antigen as identified according to the method defined herein have been obtained, then these TCRs are engineered into immune cells, in accordance with methods well known to those of skill in the art, for use in the treatment of a medical condition.

Disclosed herein is a nucleic acid encoding a T-cell receptor, wherein the T-cell receptor encoded by the nucleic acid is capable of specifically binding to a MARK3 splice variant or fragment thereof, wherein the MARK3 splice variant or fragment thereof is bound to a HLA molecule.

Provided herein is also an isolated T-cell receptor encoded by the nucleic acid as defined herein. In some embodiments, there is provided a T-cell receptor (TCR) that specifically binds to a MARK3 splice variant, wherein the MARK3 splice variant is bound to HLA molecule. The MARK3 splice variant may be presented on the surface of an antigen-presenting cell or cancer cell.

In one embodiment, the shared antigen comprises a peptide having at least 80% sequence identity to SEQ ID NO: 1, or a nucleic acid encoding a peptide having at least 80% sequence identity to SEQ ID NO: 1.

In one embodiment, the shared antigen is a peptide having at least 80% sequence identity to SEQ ID NO: 1, or a nucleic acid encoding a peptide having at least 80% sequence identity to SEQ ID NO: 1.

Disclosed herein is a nucleic acid encoding a T-cell receptor, wherein the T-cell receptor encoded by the nucleic acid is capable of specifically binding to a NBPF9 splice variant or fragment thereof, wherein the NBPF9 splice variant or fragment thereof is bound to a HLA molecule.

Provided herein is also an isolated T-cell receptor encoded by the nucleic acid as defined herein. In some embodiments, there is provided a T-cell receptor (TCR) that specifically binds to a NBPF9 splice variant, wherein the NBPF9 splice variant is bound to HLA molecule. The NBPF9 splice variant may be presented on the surface of an antigen-presenting cell or cancer cell.

In one embodiment, the shared antigen is a peptide having at least 80% sequence identity to SEQ ID NO: 31, or a nucleic acid encoding a peptide having at least 80% sequence identity to SEQ ID NO: 31.

Disclosed herein is a nucleic acid encoding a T-cell receptor, wherein the T-cell receptor encoded by the nucleic acid is capable of specifically binding to a PARD3 splice variant or fragment thereof, wherein the PARD3 splice variant or fragment thereof is bound to a HLA molecule.

Provided herein is also an isolated T-cell receptor encoded by the nucleic acid as defined herein. In some embodiments, there is provided a T-cell receptor (TCR) that specifically binds to a PARD3 splice variant, wherein the PARD3 splice variant is bound to HLA molecule. The PARD3 splice variant may be presented on the surface of an antigen-presenting cell or cancer cell.

In one embodiment, the shared antigen is a peptide having at least 80% sequence identity to SEQ ID NO: 32, or a nucleic acid encoding a peptide having at least 80% sequence identity to SEQ ID NO: 32.

Disclosed herein is a nucleic acid encoding a T-cell receptor, wherein the T-cell receptor encoded by the nucleic acid is capable of specifically binding to a ZC3HAV1 splice variant or fragment thereof, wherein the ZC3HAV1 splice variant or fragment thereof is bound to a HLA molecule.

Provided herein is also an isolated T-cell receptor encoded by the nucleic acid as defined herein. In some embodiments, there is provided a T-cell receptor (TCR) that specifically binds to a ZC3HAV1 splice variant, wherein the ZC3HAV1 splice variant is bound to HLA molecule. The ZC3HAV1 splice variant may be presented on the surface of an antigen-presenting cell or cancer cell.

In one embodiment, the shared antigen is a peptide having at least 80% sequence identity to SEQ ID NO: 33, or a nucleic acid encoding a peptide having at least 80% sequence identity to SEQ ID NO: 33.

Disclosed herein is a nucleic acid encoding a T-cell receptor, wherein the T-cell receptor encoded by the nucleic acid is capable of specifically binding to a YAF2 splice variant or fragment thereof, wherein the YAF2 splice variant or fragment thereof is bound to a HLA molecule.

Provided herein is also an isolated T-cell receptor encoded by the nucleic acid as defined herein. In some embodiments, there is provided a T-cell receptor (TCR) that specifically binds to a YAF2 splice variant, wherein the YAF2 splice variant is bound to HLA molecule. The YAF2 splice variant may be presented on the surface of an antigen-presenting cell or cancer cell.

In one embodiment, the shared antigen is a peptide having at least 80% sequence identity to SEQ ID NO: 34, or a nucleic acid encoding a peptide having at least 80% sequence identity to SEQ ID NO: 34.

Disclosed herein is a nucleic acid encoding a T-cell receptor, wherein the T-cell receptor encoded by the nucleic acid is capable of specifically binding to a CAMKK1 splice variant or fragment thereof, wherein the CAMKK1 splice variant or fragment thereof is bound to a HLA molecule.

Provided herein is also an isolated T-cell receptor encoded by the nucleic acid as defined herein. In some embodiments, there is provided a T-cell receptor (TCR) that specifically binds to a CAMKK1 splice variant, wherein the CAMKK1 splice variant is bound to HLA molecule. The CAMKK1 splice variant may be presented on the surface of an antigen-presenting cell or cancer cell.

In one embodiment, the shared antigen is a peptide having at least 80% sequence identity to SEQ ID NO: 35, or a nucleic acid encoding a peptide having at least 80% sequence identity to SEQ ID NO: 35.

Disclosed herein is a nucleic acid encoding a T-cell receptor, wherein the T-cell receptor encoded by the nucleic acid is capable of specifically binding to a LRR1 splice variant or fragment thereof, wherein the LRR1 splice variant or fragment thereof is bound to a HLA molecule.

Provided herein is also an isolated T-cell receptor encoded by the nucleic acid as defined herein. In some embodiments, there is provided a T-cell receptor (TCR) that specifically binds to a LRR1 splice variant, wherein the LRR1 splice variant is bound to HLA molecule. The LRR1 splice variant may be presented on the surface of an antigen-presenting cell or cancer cell.

In one embodiment, the shared antigen is a peptide having at least 80% sequence identity to SEQ ID NO: 36 or SEQ ID NO: 51, or a nucleic acid encoding a peptide having at least 80% sequence identity to SEQ ID NO: 36 or SEQ ID NO: 51.

Disclosed herein is a nucleic acid encoding a T-cell receptor, wherein the T-cell receptor encoded by the nucleic acid is capable of specifically binding to a ZNF670 splice variant or fragment thereof, wherein the ZNF670 splice variant or fragment thereof is bound to a HLA molecule.

Provided herein is also an isolated T-cell receptor encoded by the nucleic acid as defined herein. In some embodiments, there is provided a T-cell receptor (TCR) that specifically binds to a ZNF670 splice variant, wherein the ZNF670 splice variant is bound to HLA molecule. The ZNF670 splice variant may be presented on the surface of an antigen-presenting cell or cancer cell.

In one embodiment, the shared antigen is a peptide having at least 80% sequence identity to SEQ ID NO: 37, or a nucleic acid encoding a peptide having at least 80% sequence identity to SEQ ID NO: 37.

Disclosed herein is a nucleic acid encoding a T-cell receptor, wherein the T-cell receptor encoded by the nucleic acid is capable of specifically binding to a GRINA splice variant or fragment thereof, wherein the GRINA splice variant or fragment thereof is bound to a HLA molecule.

Provided herein is also an isolated T-cell receptor encoded by the nucleic acid as defined herein. In some embodiments, there is provided a T-cell receptor (TCR) that specifically binds to a GRINA splice variant, wherein the GRINA splice variant is bound to HLA molecule. The GRINA splice variant may be presented on the surface of an antigen-presenting cell or cancer cell.

In one embodiment, the shared antigen is a peptide having at least 80% sequence identity to SEQ ID NO: 38, or a nucleic acid encoding a peptide having at least 80% sequence identity to SEQ ID NO: 38.

Disclosed herein is a nucleic acid encoding a T-cell receptor, wherein the T-cell receptor encoded by the nucleic acid is capable of specifically binding to a MZF1 splice variant or fragment thereof, wherein the MZF1 splice variant or fragment thereof is bound to a HLA molecule.

Provided herein is also an isolated T-cell receptor encoded by the nucleic acid as defined herein. In some embodiments, there is provided a T-cell receptor (TCR) that specifically binds to a MZF1 splice variant, wherein the MZF1 splice variant is bound to HLA molecule. The MZF1 splice variant may be presented on the surface of an antigen-presenting cell or cancer cell.

In one embodiment, the shared antigen is a peptide having at least 80% sequence identity to SEQ ID NO: 52, or a nucleic acid encoding a peptide having at least 80% sequence identity to SEQ ID NO: 52.

In some embodiments, the TCR comprises a TCR α chain domain comprising a TRAV6*01 amino acid sequence, a TRAJ9*01 amino acid sequence and/or a CDR3 that has at least 70% sequence identity to an amino acid sequence of SEQ ID NO: 20. In some embodiments, the TCR comprises a TCR β chain domain comprising a TRBV7-9*01 amino acid sequence, a TRBJ1-2*01 amino acid sequence and/or a CDR3 that has at least 70% sequence identity to an amino acid sequence of SEQ ID NO: 28.

In some embodiments, the TCR comprises a) a TCR α chain domain comprising a TRAV6*01 amino acid sequence, a TRAJ9*01 amino acid sequence and/or a CDR3 that has at least 70% sequence identity to an amino acid sequence of SEQ ID NO: 20; and b) a TCR β chain domain comprising a TRBV7-9*01 amino acid sequence, a TRBJ1-2*01 amino acid sequence and/or a CDR3 that has at least 70% sequence identity to an amino acid sequence of SEQ ID NO: 28

In some embodiments, the TCR comprises a) a TCR α chain domain comprising an amino acid sequence that has at least 70% sequence identity to SEQ ID NO: 15, an amino acid sequence that has at least 70% sequence identity to SEQ ID NO; 16 and an amino acid that has at least 70% sequence identity to SEQ ID NO: 20; and b) a TCR β chain domain comprising an amino acid sequence that has at least 70% sequence identity to SEQ ID NO: 23, an amino acid sequence that has at least 70% sequence identity to SEQ ID NO; 24 and an amino acid that has at least 70% sequence identity to SEQ ID NO: 28.

The TCR may comprise a) a TCR α chain domain comprising i) a CDR1 sequence of SEQ ID NO: 17, ii) a CDR2 sequence of SEQ ID NO: 18 and/or iii) a CDR3 of SEQ ID NO: 20. The TCR may comprise b) a TCR β chain domain comprising i) a CDR1 sequence of SEQ ID NO: 25, ii) a CDR2 sequence of SEQ ID NO: 26 and/or iii) a CDR3 sequence of SEQ ID NO: 28.

In some embodiments, there is provided a TCR comprising a) a TCR α chain variable domain comprising a sequence having at least 70% (or 80%, 90%, 95% or 100%) sequence identity to SEQ ID NO: 21, and b) a TCR β chain variable domain comprising a sequence having at least 70% (or 80%, 90%, 95% or 100%) sequence identity to SEQ ID NO:29.

In some embodiments, there is provided a TCR comprising a) a TCR α chain domain comprising a sequence having at least 70% (or 80%, 90%, 95% or 100%) sequence identity to SEQ ID NO: 22, and b) a TCR β chain domain comprising a sequence having at least 70% (or 80%, 90%, 95% or 100%) sequence identity to SEQ ID NO:30.

The invention also provides a cell harbouring a TCR expression vector. The vector may comprise nucleic acid of the invention encoding in a single open reading frame, or two distinct open reading frames, the alpha chain and the beta chain respectively.

Also provided is a cell harbouring a first expression vector which comprises nucleic acid encoding the alpha chain of a TCR as defined herein, and a second expression vector which comprises nucleic acid encoding the beta chain of a TCR as defined herein.

Soluble TCR for Immunotherapy

The identified TCR sequence can be solubilized by removal of the transmembrane region and cytoplasmic tail of the TCR chains. The interchain stability of the soluble TCR can be stabilized by modifications of the sequences of the TCR chains; for example, residues in TRA and TRB chains can be replaced with cysteine which allow disulphide bonds to be formed between the two chains. These soluble TCRs may be further modified to have additional functionalities that enhance treatment efficacy; for example, fusion to an anti-CD3 single chain variable fragment, which allows recruitment of CD3 T cells. Such methods are well known to persons of skill in the art and can be found, for example, in Walseng et al Plos One 2015 and Damato et al Cancers (Basel) 2019.

Alternatively, identified TCR sequences may be produced recombinantly by expressing a nucleotide sequence encoding the variable regions of the TCR in a host cell (such as in mammalian Chinese Hamster Ovary cells). With the aid of an expression vector, a nucleic acid containing the nucleotide sequence may be transfected and expressed in a host cell suitable for the production of a soluble TCR.

Provided herein is a solubilised TCR that binds specifically to a shared antigen as defined herein, wherein the shared antigen is bound to a HLA molecule and optionally presented on the surface of an antigen-presenting cell.

Provided herein is a solubilised TCR that binds specifically to a MARK3 splice variant, wherein the MARK3 splice variant is bound to a HLA molecule and optionally presented on the surface of a cancer cell.

Provided herein is a solubilised TCR that binds specifically to a MARK3 splice variant, wherein the MARK3 splice variant is bound to a HLA molecule and optionally presented on the surface of a cancer cell, wherein the TCR comprises a) a TCR α chain variable domain comprising a sequence having at least 70% sequence identity to SEQ ID NO: 21, and b) a TCR β chain variable domain comprising a sequence having at least 70% sequence identity to SEQ ID NO:29.

In one embodiment, the solubilised TCR comprises a) a TCR α chain domain comprising i) a CDR1 sequence of SEQ ID NO: 17, ii) a CDR2 sequence of SEQ ID NO: 18 and iii) a CDR3 of SEQ ID NO: 20; and b) a TCR β chain domain comprising i) a CDR1 sequence of SEQ ID NO: 25, ii) a CDR2 sequence of SEQ ID NO: 26 and/or iii) a CDR3 sequence of SEQ ID NO: 28.

Provided herein is a solubilised TCR that binds specifically to a NBPF9 splice variant, wherein the NBPF9 splice variant is bound to a HLA molecule and optionally presented on the surface of a cancer cell.

Provided herein is a solubilised TCR that binds specifically to a PARD3 splice variant, wherein the PARD3 splice variant is bound to a HLA molecule and optionally presented on the surface of a cancer cell.

Provided herein is a solubilised TCR that binds specifically to a ZC3HAV1 splice variant, wherein the ZC3HAV1 splice variant is bound to a HLA molecule and optionally presented on the surface of a cancer cell.

Provided herein is a solubilised TCR that binds specifically to a YAF2 splice variant, wherein the YAF2 splice variant is bound to a HLA molecule and optionally presented on the surface of a cancer cell.

Provided herein is a solubilised TCR that binds specifically to a CAMKK1 splice variant, wherein the CAMKK1 splice variant is bound to a HLA molecule and optionally presented on the surface of a cancer cell.

Provided herein is a solubilised TCR that binds specifically to a LRR1 splice variant, wherein the LRR1 splice variant is bound to a HLA molecule and optionally presented on the surface of a cancer cell.

Provided herein is a solubilised TCR that binds specifically to a ZNF670 splice variant, wherein the ZNF670 splice variant is bound to a HLA molecule and optionally presented on the surface of a cancer cell.

Provided herein is a solubilised TCR that binds specifically to a GRINA splice variant, wherein the GRINA splice variant is bound to a HLA molecule and optionally presented on the surface of a cancer cell.

Provided herein is a solubilised TCR that binds specifically to a MZF1 splice variant, wherein the MZF1 splice variant is bound to a HLA molecule and optionally presented on the surface of a cancer cell.

In some embodiments, there is provided a solubilised TCR that is fused to an antibody such as a single chain variable fragment. In some embodiments, the single chain variable fragment is an anti-CD3 single chain variable fragment.

The solubilised TCRs of the present invention can also be attached to a detectable label (such as fluorescent labels, radiolabels, enzymes, nucleic acid probes) or a therapeutic agent (such as an immunomodulatory, radioactive isotope, toxin, enzyme or a cytotoxic agent).

The solubilised TCRs as defined herein may be glycosylated. The degree of glycosylation may be controlled in vivo, by using particular cell lines for example, or in vitro, by chemical modification. Such modifications are desirable, since glycosylation can improve pharmacokinetics, reduce immunogenicity and more closely mimic a native human protein.

Engineered Immune Cells

Provided herein is an engineered immune cell comprising a nucleic acid or expression vector encoding a T-cell receptor as defined herein, wherein the engineered immune cell is capable of specifically binding to a shared antigen or fragment thereof, wherein the shared antigen or fragment thereof is bound to a HLA molecule and optionally presented on the surface of an antigen-presenting cell or cancer cell. The engineered immune cell may be a T cell or a NK cell. In some embodiments, the engineered immune cell may be a mixture of T lymphocytes. In another embodiment the engineered cell is an allogeneic cell that is compatible with the patient being treated.

Treatment

Disclosed herein is a method of treating a medical condition, the method comprising administering a solubilised TCR as defined herein or an engineered immune cell expressing a T-cell receptor (TCR) targeting a shared antigen identified according to a method as defined herein to a subject to treat the medical condition in the subject, wherein the shared antigen is bound to a HLA molecule. There is also provided a solubilised TCR or an engineered immune cell for use in treatment of the medical condition. Also provided is the use of a solubilised TCR or an engineered immune cell as defined herein in the manufacture of a medicament for the treatment of the medical condition.

Disclosed herein is a method of treating a cancer associated with the expression of a MARK3 splice variant, the method comprising administering a solubilised TCR or an engineered immune cell expressing a T-cell receptor (TCR) as defined herein to a subject to treat the cancer in the subject.

Disclosed herein is a method of treating a cancer associated with the expression of a MARK3 splice variant, the method comprising administering a solubilised TCR that specifically binds to MARK3 splice variant or an engineered immune cell expressing a T-cell receptor (TCR) that specifically binds to MARK3 splice variant that is bound to a HLA molecule, wherein the solubilised TCR or TCR comprises a) a TCR α chain variable domain comprising a sequence having at least 70% sequence identity to a SEQ ID NO: 21, and b) a TCR β chain variable domain comprising a sequence having at least 70% sequence identity to SEQ ID NO: 29.

In one embodiment, the shared antigen is a peptide having at least 80% sequence identity to SEQ ID NO: 1, or a nucleic acid encoding a peptide having at least 80% sequence identity to SEQ ID NO: 1.

Disclosed herein is a method of treating a cancer associated with the expression of a MARK3 splice variant, the method comprising administering a solubilised TCR that specifically binds to MARK3 splice variant or an engineered immune cell expressing a T-cell receptor (TCR) that specifically binds to MARK3 splice variant that is bound to a HLA molecule, wherein the solubilised TCR or TCR comprises a) a TCR α chain domain comprising a sequence having at least 70% sequence identity to SEQ ID NO: 22, and b) a TCR β chain domain comprising a sequence having at least 70% sequence identity to SEQ ID NO:30.

Provided herein is an engineered immune cell for use in treatment of a cancer associated with the expression of a MARK3 splice variant, wherein the immune cell expresses a T-cell receptor (TCR) as defined herein to a subject to treat the cancer in the subject

There is also provided an engineered immune cell for use in treatment of a cancer associated with the expression of a MARK3 splice variant, wherein the immune cell expresses a T-cell receptor (TCR) that specifically binds to MARK3 splice variant that is bound to a HLA molecule, wherein the TCR comprises a) a TCR α chain variable domain comprising a sequence having at least 70% sequence identity to SEQ ID NO: 21, and b) a TCR β chain variable domain comprising a sequence having at least 70% sequence identity to SEQ ID NO:29.

Provided herein is the use of an engineered immune cell in the manufacture of a medicament for the treatment of a cancer associated with the expression of a MARK3 splice variant, wherein the immune cell expresses a T-cell receptor (TCR) as defined herein to a subject to treat the cancer in the subject

Also provided is the use of an engineered immune cell in the manufacture of a medicament for the treatment of the medical condition; wherein the immune cell expresses a T-cell receptor (TCR) that specifically binds to MARK3 splice variant that is bound to a HLA molecule, wherein the TCR comprises a) a TCR α chain variable domain comprising a sequence having at least 70% sequence identity to SEQ ID NO: 21, and b) a TCR β chain variable domain comprising a sequence having at least 70% sequence identity to SEQ ID NO:29.

Disclosed herein is a method of treating a cancer associated with the expression of a NBPF9 splice variant, the method comprising administering a solubilised TCR as defined herein or an engineered immune cell expressing a T-cell receptor (TCR) as defined herein to a subject to treat the cancer in the subject.

Provided herein is a solubilised TCR or an engineered immune cell as defined herein for use in treatment of a cancer associated with the expression of a NBPF9 splice variant.

Provided herein is the use of an a solubilised TCR or an engineered immune cell as defined herein in the manufacture of a medicament for the treatment of a cancer associated with the expression of a NBPF9 splice variant.

Disclosed herein is a method of treating a cancer associated with the expression of a PARD3 splice variant, the method comprising administering a solubilised TCR or an engineered immune cell expressing a T-cell receptor (TCR) as defined herein to a subject to treat the cancer in the subject.

Provided herein is a solubilised TCR or an engineered immune cell for use in treatment of a cancer associated with the expression of a PARD3 splice variant.

Provided herein is the use of a solubilised TCR or an engineered immune cell in the manufacture of a medicament for the treatment of a cancer associated with the expression of a PARD3 splice variant.

Disclosed herein is a method of treating a cancer associated with the expression of a ZC3HAV1 splice variant, the method comprising administering a solubilised TCR or an engineered immune cell expressing a T-cell receptor (TCR) as defined herein to a subject to treat the cancer in the subject.

Provided herein is a solubilised TCR or an engineered immune cell for use in treatment of a cancer associated with the expression of a ZC3HAV1 splice variant.

Provided herein is the use of a solubilised TCR or an engineered immune cell in the manufacture of a medicament for the treatment of a cancer associated with the expression of a ZC3HAV1 splice variant.

Disclosed herein is a method of treating a cancer associated with the expression of a YAF2 splice variant, the method comprising administering a solubilised TCR or an engineered immune cell expressing a T-cell receptor (TCR) as defined herein to a subject to treat the cancer in the subject.

Provided herein is a solubilised TCR or an engineered immune cell for use in treatment of a cancer associated with the expression of a YAF2 splice variant.

Provided herein is the use of a solubilised TCR or an engineered immune cell in the manufacture of a medicament for the treatment of a cancer associated with the expression of a YAF2 splice variant.

Disclosed herein is a method of treating a cancer associated with the expression of a CAMKK1 splice variant, the method comprising administering a solubilised TCR or an engineered immune cell expressing a T-cell receptor (TCR) as defined herein to a subject to treat the cancer in the subject.

Provided herein is a solubilised TCR or an engineered immune cell for use in treatment of a cancer associated with the expression of a CAMKK1 splice variant.

Provided herein is the use of a solubilised TCR or an engineered immune cell in the manufacture of a medicament for the treatment of a cancer associated with the expression of a CAMKK1 splice variant.

Disclosed herein is a method of treating a cancer associated with the expression of a LRR1 splice variant, the method comprising administering a solubilised TCR or an engineered immune cell expressing a T-cell receptor (TCR) as defined herein to a subject to treat the cancer in the subject.

Provided herein is a solubilised TCR or an engineered immune cell for use in treatment of a cancer associated with the expression of a LRR1 splice variant.

Provided herein is the use of a solubilised TCR or an engineered immune cell in the manufacture of a medicament for the treatment of a cancer associated with the expression of a LRR1 splice variant.

Disclosed herein is a method of treating a cancer associated with the expression of a ZNF670 splice variant, the method comprising administering a solubilised TCR or an engineered immune cell expressing a T-cell receptor (TCR) as defined herein to a subject to treat the cancer in the subject.

Provided herein is a solubilised TCR or an engineered immune cell for use in treatment of a cancer associated with the expression of a ZNF670 splice variant.

Provided herein is the use of a solubilised TCR or an engineered immune cell in the manufacture of a medicament for the treatment of a cancer associated with the expression of a ZNF670 splice variant.

Disclosed herein is a method of treating a cancer associated with the expression of a GRINA splice variant, the method comprising administering a solubilised TCR or an engineered immune cell expressing a T-cell receptor (TCR) as defined herein to a subject to treat the cancer in the subject.

Provided herein is a solubilised TCR or an engineered immune cell for use in treatment of a cancer associated with the expression of a GRINA splice variant.

Provided herein is the use of a solubilised TCR or an engineered immune cell in the manufacture of a medicament for the treatment of a cancer associated with the expression of a GRINA splice variant.

Disclosed herein is a method of treating a cancer associated with the expression of a MZF1 splice variant, the method comprising administering a solubilised TCR or an engineered immune cell expressing a T-cell receptor (TCR) as defined herein to a subject to treat the cancer in the subject.

Provided herein is a solubilised TCR or an engineered immune cell for use in treatment of a cancer associated with the expression of a MZF1 splice variant.

Provided herein is the use of a solubilised TCR or an engineered immune cell in the manufacture of a medicament for the treatment of a cancer associated with the expression of a MZF1 splice variant.

Pharmaceutical Compositions

Provided herein is a pharmaceutical composition comprising an antibody, a solubilised TCR, an engineered immune cell (such as T cell or NK cell) expressing a T-cell receptor (TCR) or an expanded immune cell (such as T cell or NK cell) population as defined herein. The antibody, solubilised TCR, engineered immune cell or expanded immune cell population as defined herein are preferably used in such a pharmaceutical composition, in doses mixed with an acceptable carrier or carrier material, such that the disease can be treated or at least alleviated. Such a composition can (in addition to the active component and the carrier) include filling material, salts, buffer, stabilizers, solubilisers and other materials, which are known state of the art. The pharmaceutical composition may, for example, be an injectable composition.

The term “pharmaceutically acceptable” defines a non-toxic material, which does not interfere with effectiveness of the biological activity of the active component. The choice of the carrier is dependent on the application.

The pharmaceutical composition may contain additional components which enhance the activity of the active component or which supplement the treatment. Such additional components and/or factors can be part of the pharmaceutical composition to achieve synergistic effects or to minimize adverse or unwanted effects.

Techniques for the formulation or preparation and application/medication of active components of the present invention are published in “Remington's Pharmaceutical Sciences”, Mack Publishing Co., Easton, PA, latest edition. An appropriate application is a parenteral application, for example intramuscular, subcutaneous, intramedular injections as well as intrathecal, direct intraventricular, intravenous, intranodal, intraperitoneal or intratumoral injections. The intravenous injection is the preferred treatment of a patient.

In some embodiments, the pharmaceutical composition is an infusion or an injection.

An injectable composition is a pharmaceutically acceptable fluid composition comprising at least one active ingredient, e.g. an expanded immune cell population (for example autologous or allogenic to the patient to be treated) expressing a TCR. The active ingredient is usually dissolved or suspended in a physiologically acceptable carrier, and the composition can additionally comprise minor amounts of one or more non-toxic auxiliary substances, such as emulsifying agents, preservatives, and pH buffering agents and the like. Such injectable compositions that are useful for use with the fusion proteins of this disclosure are conventional: appropriate formulations are well known to those of ordinary skill in the art.

Typically, the pharmaceutical composition comprises at least one pharmaceutically acceptable carrier.

Antibodies for Immunotherapy

Provided herein are methods of producing an antibody that binds specifically to a shared antigen or fragment thereof. The shared antigen or fragment thereof may be bound to a HLA molecule and may optionally be presented on the surface of the antigen-presenting cell or the cancer cell.

By “antibody” is meant a molecule that has binding affinity for a target antigen (shared antigen). It will be understood that this term extends to immunoglobulins, immunoglobulin fragments and non-immunoglobulin derived protein frameworks that exhibit antigen-binding activity. Representative antigen-binding molecules that are useful in the practice of the present invention include polyclonal and monoclonal antibodies as well as their fragments (such as Fab, Fab′, F(ab′)2, Fv), single chain (scFv) and domain antibodies (including, for example, shark and camelid antibodies), and fusion proteins comprising an antibody, and any other modified configuration of the immunoglobulin molecule that comprises an antigen binding/recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes.

There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and μ, respectively. The subunit structures and three-dimensional configurations of different classes of immunoglobulins are well known. Antigen-binding molecules also encompass dimeric antibodies, as well as multivalent forms of antibodies. In some embodiments, the antigen-binding molecules are chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, for example, U.S. Pat. No. 4,816,567; and Morrison et al., 1984, Proc. Natl. Acad. Sci. USA 81:6851-6855). Also contemplated, are humanized antibodies, which are generally produced by transferring complementarity determining regions (CDRs) from heavy and light variable chains of a non-human (e.g., rodent, preferably mouse) immunoglobulin into a human variable domain. Typical residues of human antibodies are then substituted in the framework regions of the non-human counterparts. The use of antibody components derived from humanized antibodies obviates potential problems associated with the immunogenicity of non-human constant regions. General techniques for cloning non-human, particularly murine, immunoglobulin variable domains are described, for example, by Orlandi et al. (1989, Proc. Natl. Acad. Sci. USA 86: 3833). Techniques for producing humanized monoclonal antibodies are described, for example, by Jones et al. (1986, Nature 321:522), Carter et al. (1992, Proc. Natl. Acad. Sci. USA 89: 4285), Sandhu (1992, Crit. Rev. Biotech. 12: 437), Singer et al. (1993, J. Immun. 150: 2844), Sudhir (ed., Antibody Engineering Protocols, Humana Press, Inc. 1995), Kelley (“Engineering Therapeutic Antibodies,” in Protein Engineering: Principles and Practice Cleland et al. (eds.), pages 399-434 (John Wiley & Sons, Inc. 1996), and by Queen et al., U.S. Pat. No. 5,693,762 (1997). Humanized antibodies include “primatized” antibodies in which the antigen-binding region of the antibody is derived from an antibody produced by immunizing macaque monkeys with the antigen of interest. Also contemplated as antigen-binding molecules are humanized antibodies.

In some embodiments, there is provided a method of producing an antibody, the method comprising: 1) immunizing an animal with a shared antigen or fragment thereof identified according to a method as defined herein; 2) identifying and/or isolating a B cell from the animal, which binds specifically to the shared antigen or fragment thereof; and 3) producing the antibody expressed by that B cell. The shared antigen or fragment thereof may be bound to a HLA molecule and/or may be presented on the surface of an antigen-presenting cell or cancer cell. Disclosed herein is also an antibody that is obtained according to a method as defined herein.

In some embodiments, there is provided a method of producing an antibody, the method comprising: 1) immunizing an animal with a MARK3 splice variant peptide; 2) identifying and/or isolating a B cell from the animal, which binds specifically to the MARK3 splice variant; and 3) producing the antibody expressed by that B cell.

In some embodiments, there is provided a method of producing an antibody, the method comprising: 1) immunizing an animal with a NBPF9 splice variant peptide; 2) identifying and/or isolating a B cell from the animal, which binds specifically to the NBPF9 splice variant; and 3) producing the antibody expressed by that B cell.

In some embodiments, there is provided a method of producing an antibody, the method comprising: 1) immunizing an animal with a PARD3 splice variant peptide; 2) identifying and/or isolating a B cell from the animal, which binds specifically to the PARD3 splice variant; and 3) producing the antibody expressed by that B cell.

In some embodiments, there is provided a method of producing an antibody, the method comprising: 1) immunizing an animal with a ZC3HAV1 splice variant peptide; 2) identifying and/or isolating a B cell from the animal, which binds specifically to the ZC3HAV1 splice variant; and 3) producing the antibody expressed by that B cell.

In some embodiments, there is provided a method of producing an antibody, the method comprising: 1) immunizing an animal with a YAF2 splice variant peptide; 2) identifying and/or isolating a B cell from the animal, which binds specifically to the YAF2 splice variant; and 3) producing the antibody expressed by that B cell.

In some embodiments, there is provided a method of producing an antibody, the method comprising: 1) immunizing an animal with a CAMKK1 splice variant peptide; 2) identifying and/or isolating a B cell from the animal, which binds specifically to the CAMKK1 splice variant; and 3) producing the antibody expressed by that B cell.

In some embodiments, there is provided a method of producing an antibody, the method comprising: 1) immunizing an animal with a LRR1 splice variant peptide; 2) identifying and/or isolating a B cell from the animal, which binds specifically to the LRR1 splice variant; and 3) producing the antibody expressed by that B cell.

In some embodiments, there is provided a method of producing an antibody, the method comprising: 1) immunizing an animal with a ZNF670 splice variant peptide; 2) identifying and/or isolating a B cell from the animal, which binds specifically to the ZNF670 splice variant; and 3) producing the antibody expressed by that B cell.

In some embodiments, there is provided a method of producing an antibody, the method comprising: 1) immunizing an animal with a GRINA splice variant peptide; 2) identifying and/or isolating a B cell from the animal, which binds specifically to the GRINA splice variant; and 3) producing the antibody expressed by that B cell.

In some embodiments, there is provided a method of producing an antibody, the method comprising: 1) immunizing an animal with a MZF1 splice variant peptide; 2) identifying and/or isolating a B cell from the animal, which binds specifically to the MZF1 splice variant; and 3) producing the antibody expressed by that B cell.

Methods for producing antibodies are well known to persons of skill in the art. One such method comprises screening a population of B cells to generate a B cell library enriched in B cells capable of binding specifically to the shared antigen; amplifying cDNA obtained from mRNA expressed in a single B cell or a plurality of B cells in the B cell library to prepare an immunoglobulin library comprising Vₕ and Vₗ domains; cloning the immunoglobulin library into an expression vector to form a library of expression vectors capable of expressing the Vₕ and Vi domains, whereby the Vₕ and Vₗ domains are naturally paired; using the library of expression vectors to express the Vₕ and Vₗ domains in an expression system to form an antibody library, wherein the antibodies comprise naturally paired Vₕ and Vₗ domains; and screening the antibody library for binding to the HLA-binding peptide.

Alternatively, antibody sequences that bind to a shared antigen or fragment thereof that is bound to the HLA molecule may be identified through screening a library of yeast or bacteriophages expressing antibodies on their surface. This involves identifying which antibody sequence, expressed by individual clones of yeast or bacteriophage, is able to bind to a shared antigen or fragment thereof that is bound to the HLA molecule. Identification of antibody sequences may be done by incubating the bacteriophage library with biotinylated HLA molecules loaded with the shared antigen and specific clones captured by streptavidin-coated magnetic beads. These methods are well known to persons of skill in the art. This may also involve mutagenesis of the antibody sequences to obtain antibodies with higher specificity and/or affinity that is able to bind to a shared antigen or fragment thereof that is bound to the HLA molecule. Multiple rounds of mutagenesis and/or identification of antibody sequences may be used to select the antibody sequence with the most ideal properties that can bind to the shared antigen or fragment thereof that is bound to the HLA molecule.

In some embodiments, there is provided a method of identifying an antibody that binds to a shared antigen or fragment thereof, the method comprising: 1) contacting a shared antigen or fragment thereof with an antibody phage display or yeast display library; wherein the shared antigen or fragment thereof is bound to a HLA molecule, 2) selecting a phage molecule or yeast cell that is bound to the shared antigen or fragment thereof; and 3) obtaining the DNA sequence of the antibody that is presented on the phage molecule or yeast cell. The method may further comprise improving the binding affinity of the antibody to the shared antigen or fragment thereof by affinity maturation methods that are well known in the art.

The antibody may be produced recombinantly by expressing a nucleotide sequence encoding the variable regions of the monoclonal antibody in a host cell (such as in mammalian Chinese Hamster Ovary cells). With the aid of an expression vector, a nucleic acid containing the nucleotide sequence may be transfected and expressed in a host cell suitable for the production. Accordingly, the antibody-based method of treating a medical condition in a subject may comprise the use of either polyclonal or monoclonal antibodies.

In one example, to express the antibodies, or antibody fragments thereof, DNAs encoding partial or full-length light and heavy chains obtained by standard molecular biology techniques are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the Vₕ segment is operatively linked to the Cₕ segment(s) within the vector and the Vₗ segment is operatively linked to the Cₗ segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

The antibodies may be further modified to have additional functionalities that enhance treatment efficacy. For example, fusion of antibody to an anti-CD3 single chain variable fragment, which allows recruitment of CD3 T cells.

In some embodiments, the antibody that binds specifically to a HLA-binding peptide or fragment thereof, can be expressed in an immune cell for treatment of a medical condition. The antibody is engineered to be embedded in the cell membrane and have a cytoplasmic tail containing domains that can activate immune cells. For example, the cytoplasmic tail may consist of the intracellular signalling domains of co-stimulatory proteins such as CD28 and 4-1BB or signalling domain of the CD3 zeta domain, such as is described in, for example, Zhang et al Sci Rep 2014. In some embodiments, the engineered immune cell may be a T cell or a NK cell. In some embodiments, the engineered immune cell may be a mixture of T lymphocytes. In another embodiment the engineered cell may be an allogeneic cell that is compatible with the patient being treated.

Disclosed herein is an antibody that binds specifically to a shared antigen identified according to a method as defined herein.

In one embodiment, the shared antigen is a peptide having at least 80% sequence identity to SEQ ID NO: 1, or is a nucleic acid encoding a peptide having at least 80% sequence identity to SEQ ID NO: 1.

Also disclosed herein is an antibody that binds specifically to a MARK3 splice variant peptide as disclosed herein.

In one embodiment, the shared antigen is a peptide having at least 80% sequence identity to SEQ ID NO: 31, or is a nucleic acid encoding a peptide having at least 80% sequence identity to SEQ ID NO: 31.

Also disclosed herein is an antibody that binds specifically to a NBPF9 splice variant peptide as disclosed herein.

In one embodiment, the shared antigen is a peptide having at least 80% sequence identity to SEQ ID NO: 32, or is a nucleic acid encoding a peptide having at least 80% sequence identity to SEQ ID NO: 32.

Also disclosed herein is an antibody that binds specifically to a PARD3 splice variant peptide as disclosed herein.

In one embodiment, the shared antigen is a peptide having at least 80% sequence identity to SEQ ID NO: 33, or is a nucleic acid encoding a peptide having at least 80% sequence identity to SEQ ID NO: 33.

Also disclosed herein is an antibody that binds specifically to a ZC3HAV1 splice variant peptide as disclosed herein.

In one embodiment, the shared antigen is a peptide having at least 80% sequence identity to SEQ ID NO: 34, or is a nucleic acid encoding a peptide having at least 80% sequence identity to SEQ ID NO: 34.

Also disclosed herein is an antibody that binds specifically to a YAF2 splice variant peptide as disclosed herein.

In one embodiment, the shared antigen is a peptide having at least 80% sequence identity to SEQ ID NO: 35, or is a nucleic acid encoding a peptide having at least 80% sequence identity to SEQ ID NO: 35.

Also disclosed herein is an antibody that binds specifically to a CAMKK1 splice variant peptide as disclosed herein.

In one embodiment, the shared antigen is a peptide having at least 80% sequence identity to SEQ ID NO: 36 or SEQ ID NO: 51, or is a nucleic acid encoding a peptide having at least 80% sequence identity to SEQ ID NO: 36 or SEQ ID NO: 51.

Also disclosed herein is an antibody that binds specifically to a LRR1 splice variant peptide as disclosed herein.

In one embodiment, the shared antigen is a peptide having at least 80% sequence identity to SEQ ID NO: 37, or is a nucleic acid encoding a peptide having at least 80% sequence identity to SEQ ID NO: 37.

Also disclosed herein is an antibody that binds specifically to a ZNF670 splice variant peptide as disclosed herein.

In one embodiment, the shared antigen is a peptide having at least 80% sequence identity to SEQ ID NO: 38, or is a nucleic acid encoding a peptide having at least 80% sequence identity to SEQ ID NO: 38.

Also disclosed herein is an antibody that binds specifically to a GRINA splice variant peptide as disclosed herein.

In one embodiment, the shared antigen is a peptide having at least 80% sequence identity to SEQ ID NO: 52, or is a nucleic acid encoding a peptide having at least 80% sequence identity to SEQ ID NO: 52.

Also disclosed herein is an antibody that binds specifically to a MZF1 splice variant peptide as disclosed herein.

Disclosed herein is an antibody that binds specifically to a shared antigen identified according to a method as defined herein, wherein the shared antigen is bound to a HLA molecule and optionally presented on the surface of an antigen-presenting cell or cancer cell.

Also disclosed herein is an antibody that binds specifically to a MARK3 splice variant peptide as disclosed herein, wherein the MARK3 splice variant peptide is bound to a HLA molecule and optionally presented on the surface of an antigen-presenting cell or cancer cell.

Also disclosed herein is an antibody that binds specifically to a NBPF9 splice variant peptide as disclosed herein, wherein the NBPF9 splice variant peptide is bound to a HLA molecule and optionally presented on the surface of an antigen-presenting cell or cancer cell.

Also disclosed herein is an antibody that binds specifically to a PARD3 splice variant peptide as disclosed herein, wherein the PARD3 splice variant peptide is bound to a HLA molecule and optionally presented on the surface of an antigen-presenting cell or cancer cell.

Also disclosed herein is an antibody that binds specifically to a ZC3HAV1 splice variant peptide as disclosed herein, wherein the ZC3HAV1 splice variant peptide is bound to a HLA molecule and optionally presented on the surface of an antigen-presenting cell or cancer cell.

Also disclosed herein is an antibody that binds specifically to a YAF2 splice variant peptide as disclosed herein, wherein the YAF2 splice variant peptide is bound to a HLA molecule and optionally presented on the surface of an antigen-presenting cell or cancer cell.

Also disclosed herein is an antibody that binds specifically to a CAMKK1 splice variant peptide as disclosed herein, wherein the CAMKK1 splice variant peptide is bound to a HLA molecule and optionally presented on the surface of an antigen-presenting cell or cancer cell.

Also disclosed herein is an antibody that binds specifically to a LRR1 splice variant peptide as disclosed herein, wherein the LRR1 splice variant peptide is bound to a HLA molecule and optionally presented on the surface of an antigen-presenting cell or cancer cell.

Also disclosed herein is an antibody that binds specifically to a ZNF670 splice variant peptide as disclosed herein, wherein the ZNF670 splice variant peptide is bound to a HLA molecule and optionally presented on the surface of an antigen-presenting cell or cancer cell.

Also disclosed herein is an antibody that binds specifically to a GRINA splice variant peptide as disclosed herein, wherein the GRINA splice variant peptide is bound to a HLA molecule and optionally presented on the surface of an antigen-presenting cell or cancer cell.

Also disclosed herein is an antibody that binds specifically MZF1 splice variant peptide as disclosed herein, wherein the MZF1 splice variant peptide is bound to a HLA molecule and optionally presented on the surface of an antigen-presenting cell or cancer cell.

Also, disclosed herein is a pharmaceutical composition comprising an antibody as defined herein and a pharmaceutically acceptable carrier. The pharmaceutically acceptable carrier may be a carrier or a diluent that does not cause significant irritation to an organism and does not abrogate the biological activity and properties of the administered pharmaceutical composition. An adjuvant is included under these phrases. One of the ingredients included in the pharmaceutically acceptable carrier can be, for example, polyethylene glycol (PEG), a biocompatible polymer with a wide range of solubility in both organic and aqueous media.

Also provided herein is a method of treating cancer in a subject, the method comprising administering a pharmaceutical composition as defined herein to the subject for a sufficient time and under conditions to treat the cancer in the subject.

Provided herein is a pharmaceutical composition as defined herein for use in treating cancer in a subject.

Provided herein is the use of a pharmaceutical composition as defined herein in the manufacture of a medicament for the treatment of cancer in a subject.

Immunomodulatory Compositions

Disclosed herein is an immunomodulatory composition comprising one or more shared antigens identified according to a method as defined herein and a pharmaceutically acceptable carrier.

As used herein, the term “immunomodulatory composition” may refer to a composition that is capable of modulating the immune system of an animal. The “immunomodulatory composition” may have immunostimulatory properties that are further enhanced through modification of the protein/nucleic acid sequences and/or conjugation techniques that are familiar to a person skilled in the art. The immunomodulatory composition may comprise one or more shared antigens which are capable of stimulating the expansion of T lymphocytes and/or generating an antibody to one or more of the shared antigens, wherein the shared antigen is bound to a HLA molecule.

Also disclosed herein is an immunomodulatory composition comprising a MARK3 splice variant peptide and a pharmaceutically acceptable carrier.

In one embodiment, the MARK3 splice variant is a peptide having at least 80% sequence identity to SEQ ID NO: 1, or is a nucleic acid encoding a peptide having at least 80% sequence identity to SEQ ID NO: 1.

Also disclosed herein is an immunomodulatory composition comprising a NBPF9 splice variant peptide and a pharmaceutically acceptable carrier.

In one embodiment, the NBPF9 splice variant is a peptide having at least 80% sequence identity to SEQ ID NO: 31, or is a nucleic acid encoding a peptide having at least 80% sequence identity to SEQ ID NO: 31.

Also disclosed herein is an immunomodulatory composition comprising a PARD3 splice variant peptide and a pharmaceutically acceptable carrier.

In one embodiment, the PARD3 splice variant is a peptide having at least 80% sequence identity to SEQ ID NO: 32, or is a nucleic acid encoding a peptide having at least 80% sequence identity to SEQ ID NO: 32.

Also disclosed herein is an immunomodulatory composition comprising a ZC3HAV1 splice variant peptide and a pharmaceutically acceptable carrier.

In one embodiment, the ZC3HAV1 splice variant is a peptide having at least 80% sequence identity to SEQ ID NO: 33, or is a nucleic acid encoding a peptide having at least 80% sequence identity to SEQ ID NO: 33.

Also disclosed herein is an immunomodulatory composition comprising a YAF2 splice variant peptide and a pharmaceutically acceptable carrier.

In one embodiment, the YAF2 splice variant is a peptide having at least 80% sequence identity to SEQ ID NO: 34, or is a nucleic acid encoding a peptide having at least 80% sequence identity to SEQ ID NO: 34.

Also disclosed herein is an immunomodulatory composition comprising a CAMKK1 splice variant peptide and a pharmaceutically acceptable carrier.

In one embodiment, the CAMKK1 splice variant is a peptide having at least 80% sequence identity to SEQ ID NO: 35, or is a nucleic acid encoding a peptide having at least 80% sequence identity to SEQ ID NO: 35.

Also disclosed herein is an immunomodulatory composition comprising a LRR1 splice variant peptide and a pharmaceutically acceptable carrier.

In one embodiment, the LRR1 splice variant is a peptide having at least 80% sequence identity to SEQ ID NO: 36 or SEQ ID NO: 51, or is a nucleic acid encoding a peptide having at least 80% sequence identity to SEQ ID NO: 36 or SEQ ID NO: 51.

Also disclosed herein is an immunomodulatory composition comprising a ZNF670 splice variant peptide and a pharmaceutically acceptable carrier.

In one embodiment, the ZNF670 splice variant is a peptide having at least 80% sequence identity to SEQ ID NO: 37, or is a nucleic acid encoding a peptide having at least 80% sequence identity to SEQ ID NO: 37.

Also disclosed herein is an immunomodulatory composition comprising a GRINA splice variant peptide and a pharmaceutically acceptable carrier.

In one embodiment, the GRINA splice variant is a peptide having at least 80% sequence identity to SEQ ID NO: 38, or is a nucleic acid encoding a peptide having at least 80% sequence identity to SEQ ID NO: 38.

Also disclosed herein is an immunomodulatory composition comprising a MZF1 splice variant peptide and a pharmaceutically acceptable carrier.

In one embodiment, the MZF1 splice variant is a peptide having at least 80% sequence identity to SEQ ID NO: 52, or is a nucleic acid encoding a peptide having at least 80% sequence identity to SEQ ID NO: 52.

Disclosed herein is a method of stimulating an immune response in a subject, the method comprising administering an effective amount of an immunomodulatory composition as defined herein to the subject under conditions and for a sufficient time to stimulate the immune response in the subject.

In some embodiments, the immunomodulatory composition as defined herein comprises an adjuvant. The adjuvant is a substance that increases the immunological response of the subject to the vaccine. Suitable adjuvants include, but are not limited to, aluminium hydroxide (alum), immunostimulating complexes (ISCOMS), non-ionic block polymers or copolymers, cytokines (like IL-1, IL-2, IL-7, IFN-α, IFN-β, IFN-γ, etc.), saponins, monophosphoryl lipid A (MLA), muramyl dipeptides (MDP) and the like. Other suitable adjuvants include, for example, aluminium potassium sulphate, heat-labile or heat-stable enterotoxin isolated from Escherichia coli, cholera toxin or the B subunit thereof, diphtheria toxin, tetanus toxin, pertussis toxin, Freund's incomplete or complete adjuvant, etc. Toxin-based adjuvants, such as diphtheria toxin, tetanus toxin and pertussis toxin may be inactivated prior to use, for example, by treatment with formaldehyde.

In some embodiments, the immunomodulatory composition may comprise DNA or RNA vaccines.

In some embodiments, the immunomodulatory composition as defined herein comprises an antigen-presenting cell and one or more shared antigens. For example, dendritic cells from subject with a medical condition may be isolated and one or more shared antigens may be presented on the surface of dendritic cells ex vivo. These dendritic cells loaded with one or more shared antigens may then be administered in the subject with the medical condition to induce an immune reaction.

Provided herein is an immunomodulatory composition as defined herein for use in stimulating an immune response in a subject.

Provided herein is the use of an immunomodulatory composition as defined herein in the manufacture of a medicament for stimulating an immune response in a subject.

Provided herein is a method of treating or preventing cancer in a subject, the method comprising administering an immunomodulatory composition as defined herein to the subject to treat or prevent cancer in the subject.

Provided herein is an immunomodulatory composition as defined herein for use in preventing or treating cancer in a subject.

Provided herein is the use of an immunomodulatory composition as defined herein in the manufacture of a medicament for preventing or treating cancer in a subject.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

### EXAMPLES

### Example 1

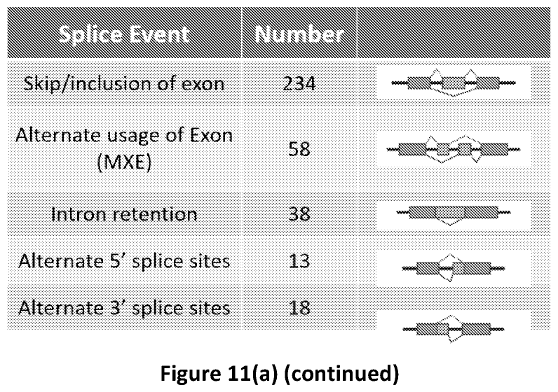

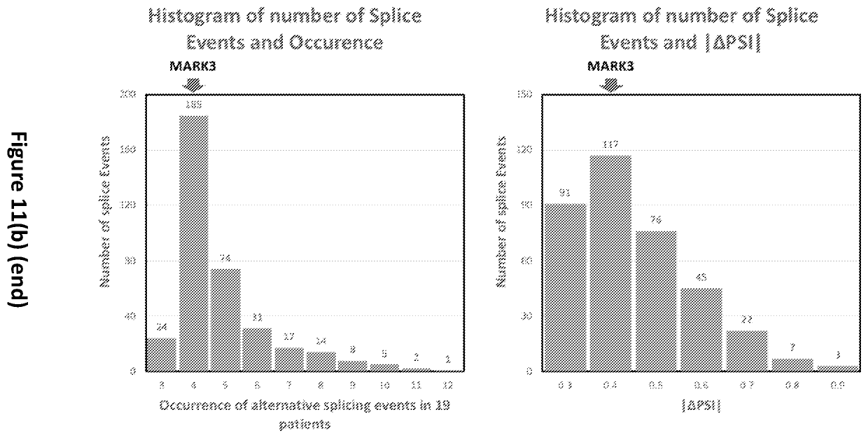

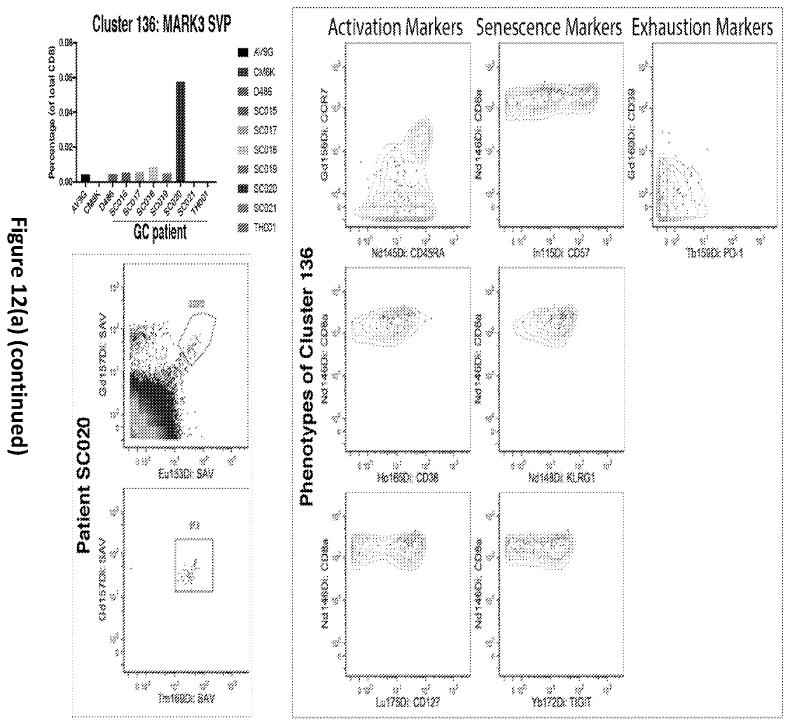

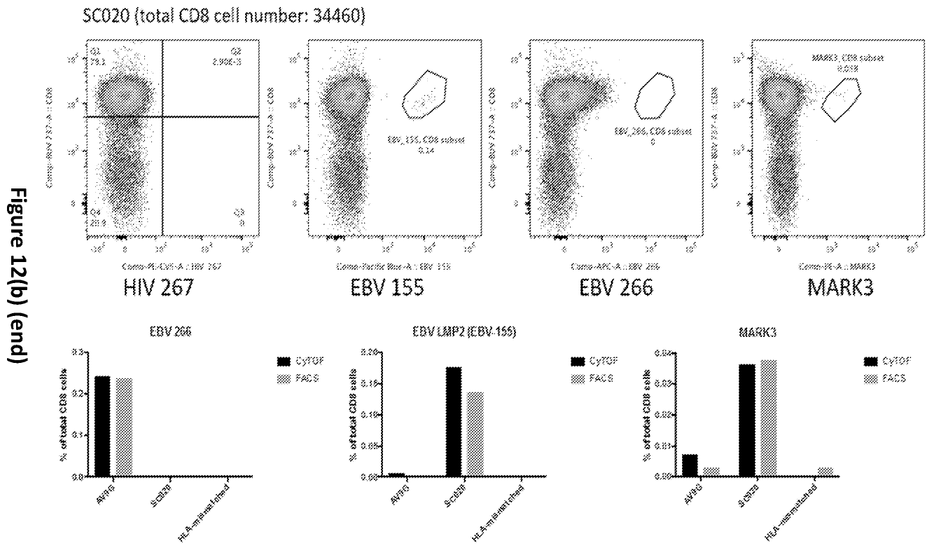

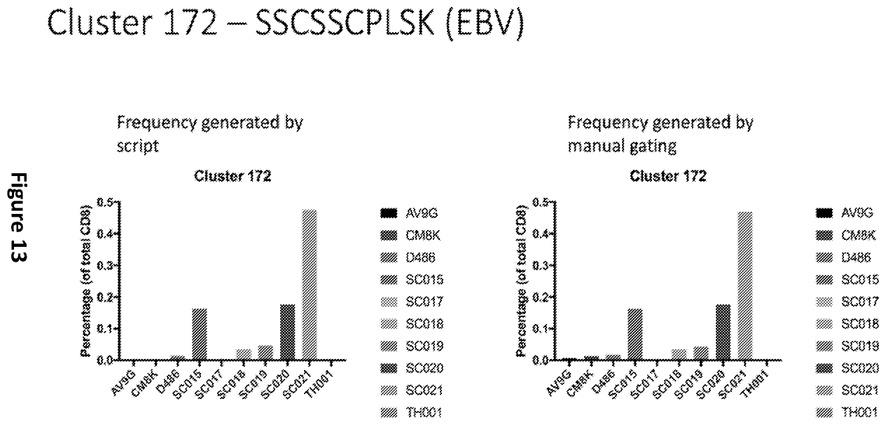

Identification of Shared Alternatively Spliced Variants in Gastric Cancer Gastric adenocarcinoma tumour samples and clinical information from 19 patients (the Discovery cohort) were obtained from the Singapore Health Services and the National University Hospital System tissue repositories. Matched normal samples were taken from non-malignant mucosa adjacent to the tumour. Deep RNA sequencing (200M reads) and mRNA splicing analysis using MISO were performed on these 19 GC patient samples. MISO analysis was performed to analyze the RNA-Seq data for differential splicing events between tumour and normal tissues. Selection criteria (Top 0.5% splicing events, at least 20% change in splicing (ΔPSI), Bayes factor >20, and occurrence in at least 3 patients) were applied to the data which yielded a list of 361 tumour-associated alternative splicing events that could lead to the identification of candidate antigenic regions which may be shared in a GC patient subpopulation (FIG. 10). A summary of the splicing alterations that were observed in gastric cancer is shown in FIG. 11(a).

Shared splice variants are identified by comparing the PSI values of individual reference and tumour samples and identifying splice variant where there are a number of tumour samples that differ significantly from the reference sample (outliers in FIG. 3). This provides the potential benefit that splice variant antigens that are not observed in populations of people are identified and are therefore genuinely novel antigens. In the analysis for the identification of shared alternatively spliced variants in gastric cancer patients, splice variants that were present in at least 3 patients out of the 19 patients were selected. A further criterion for selection of these splice variants was that the median of these outlier samples showed at least a 20% difference in the PSI value compared to the reference samples.

### Example 2

Prediction of Shared HLA-A11 Binding Peptides Derived from Alternative Splicing in Gastric Cancer

The list of 361 tumour-associated alternative splicing events was then reduced to a list of 291 tumour-associated polypeptides by selecting for splicing events that led to differences in protein sequences (i.e. coding regions only). These 291 protein regions were then used to predict 8-11 amino acid-long peptides that could bind to HLA-A11 (total of 39,876 peptides). The HLA-A11 allele was present in approximately 40% of our GC patient cohort. NetMHCpan3 was used for predicting HLA binding peptides, and it returned 153 peptides that had high affinity for HLA A11 (Rank <=0.5%). This list was further reduced to 77 peptides by removing peptides that were similar (FIG. 10). These 77 peptides corresponded to 65 genes.

### Example 3

Validation of MARK3 Splice Variant Antigen and Identification of a Shared MARK3 Splice Variant-Specific CD8+ T Cells in Gastric Cancer

To determine if GC patients have CD8+ T cells that target any of the 77 peptides that were identified in Example 2, a CyTOF screen was conducted on a new cohort of GC patients (the Validation cohort) using MHC tetramer staining of peripheral blood. The CyTOF screen was performed as described in Leong and Newell (2015). Chemically synthesized peptides were supplied by Mimotopes and were stored as dry powder at −20° C. These peptides were loaded onto biotinylated HLA-A11 by UV-mediated exchange. Streptavidin labelled with three heavy metal barcodes was bound to the peptide loaded HLA-A11 to make the HLA-tetramers used for staining peripheral blood (FIG. 7). HLA-A11 tetramer staining of 7 gastric cancer patients derived PBMCs was performed (FIG. 12(a)) and control peptides against the Epstein-Bar Virus (EBV) viral antigen were incorporated as positive control (FIG. 13). Out of these 7 samples, one of the patients, SC020, was found to be positive for expression of CD8+ T-cells capable of recognizing a peptide antigen identified in the RNA-Seq dataset from the discovery cohort (FIG. 12a). This antigen was generated from an alternative splicing event of the MARK3 gene identified in Example 2 (FIG. 11(b)). The sequence of the MARK3 splice variant antigen used in the CyTOF studies was RNMSFRIK (SEQ ID NO: 1) and the EBV peptide sequence was SSCSSCPLSK (SEQ ID NO: 2). Analyzing the CyTOF data, these MARK3-specific CD8+ T cells together with other immunophenotypic and/or lineage markers (CCR7, CD45RA, CD8a, CD38, CD127, CD57, KLRG1, TIGIT, CD39 and PD-1) revealed that these T cells are not naïve and might represent cytotoxic CD8+T Lymphocytes (CTLs) that are responding to the tumour. Patient SC020 was also positive for T Cells that reacted with the positive control peptide from the EBV (FIG. 13). The presence of the MARK3 splice variant antigen in a fresh PBMC isolate from gastric cancer patient, SC020, was further confirmed by a FACS analysis using fluorescently labelled MARK3-A11-RNMSFRIK peptide tetramers (FIG. 12(b)). The frequency of MARK3 specific CD8 T cells shows high concordance between CyTOF and FACS analysis.

### Example 4

In Silico Identification of the MARK3 Splice Variant Antigen Shared Among GC Patients

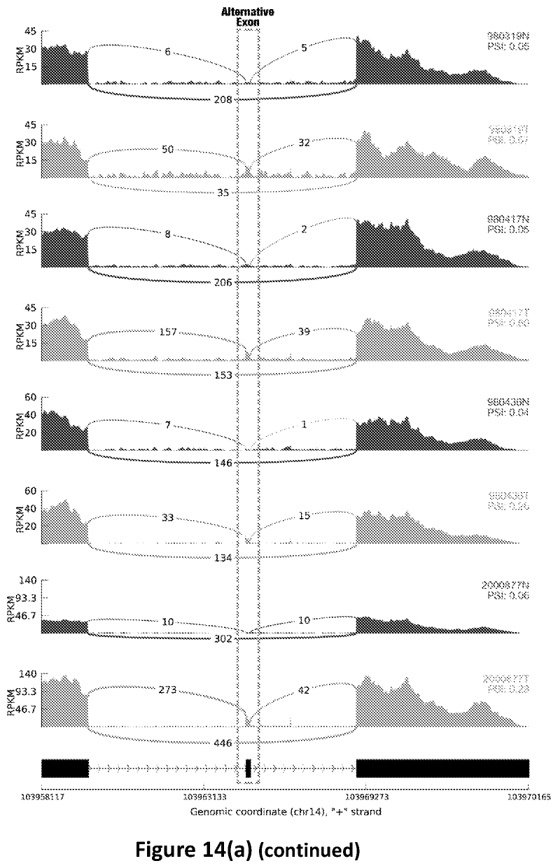

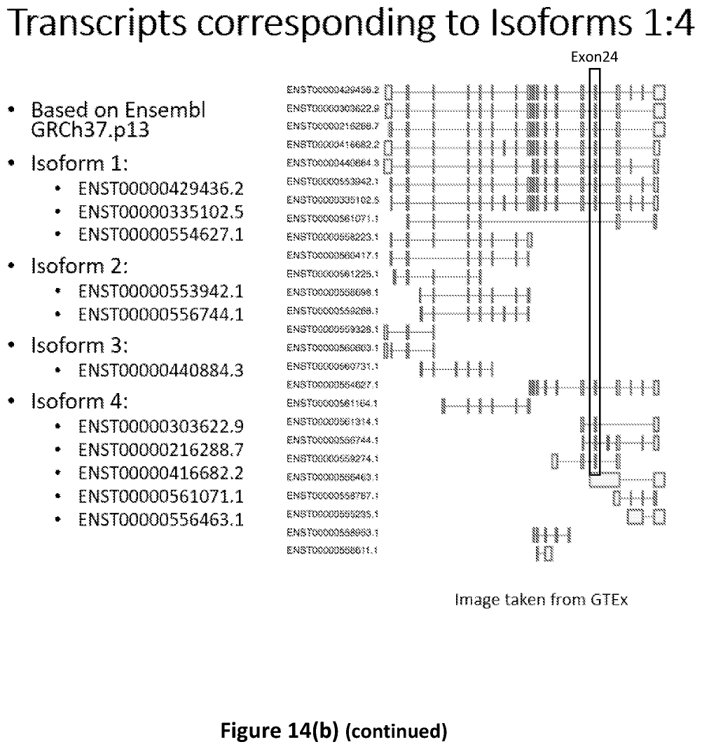

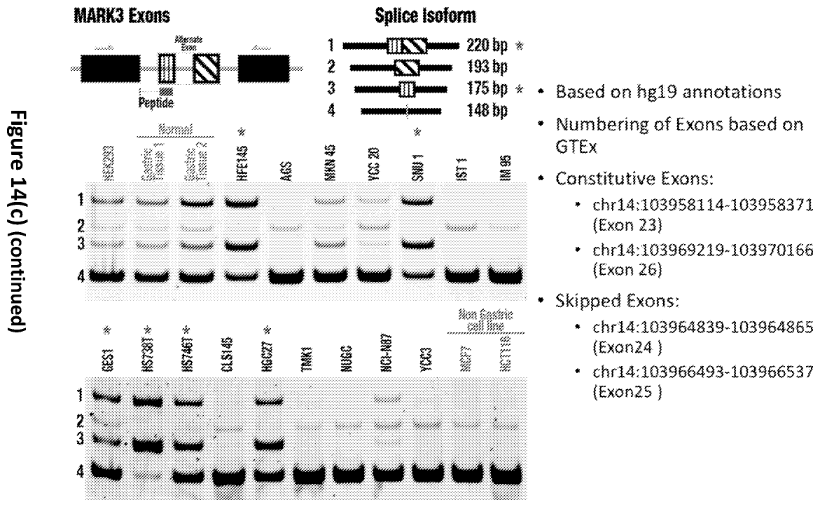

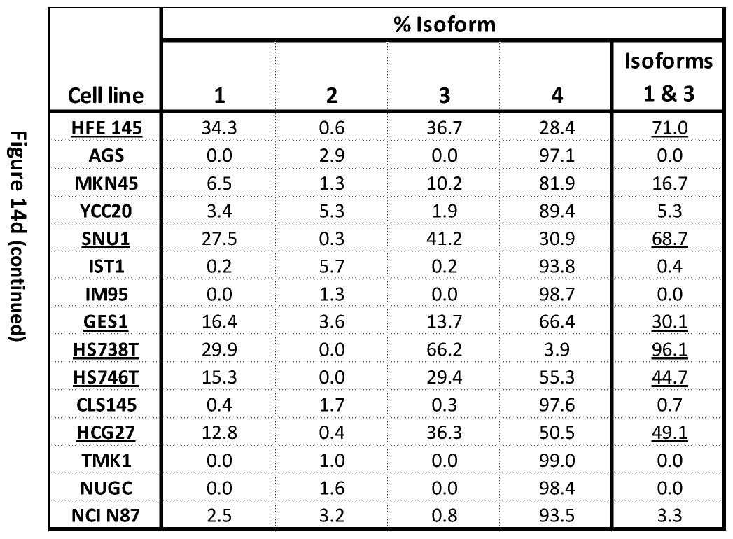

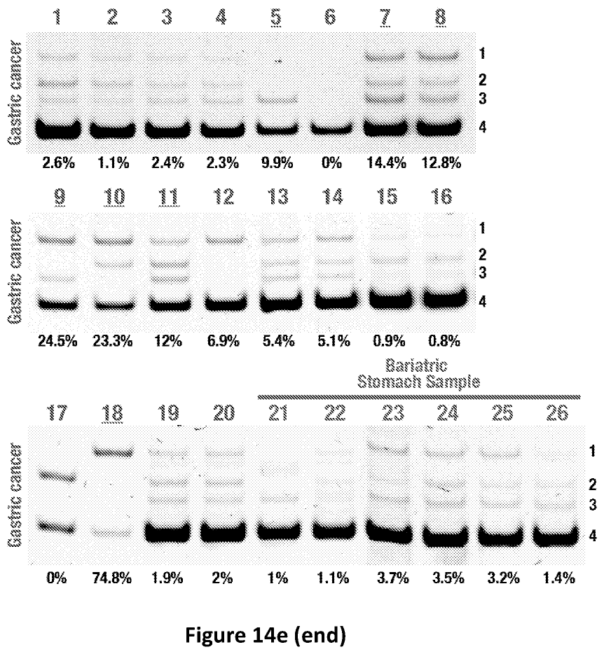

The tumour-associated alternative splicing events that corresponded to the 77 peptides were analysed to determine events which demonstrated a high frequency of being alternatively spliced and being shared amongst a GC patient subpopulation (FIG. 11(b), wherein the highest frequency is 185 occurrences). The occurrence of shared alternative splicing events varied from 3 to 12 out of the 19 GC patients. The MARK3 splice variant was found to occur in 4 out of the 19 GC patients (arrow in FIG. 11(b)). The single positive hit out of 7 samples (14%) obtained in the CyTOF screen corresponded to the frequency that was observed for the MARK3 splice variant in the discovery cohort (4/19 patients), confirming that it is a splice variant antigen shared by a GC patient subpopulation. In the four patients where aberrant splicing of MARK3 was observed, the PSI values of the normal samples were 0.05, 0.05, 0.04 and 0.06, whereas the PSI values of the tumour samples had were 0.67, 0.50, 0.26 and 0.28 (FIG. 14a). The median change in PSI was −0.335; this indicates that 33.5% of the transcripts in tumour samples contained the inclusion of an exon (Exon 24 in Table 1) that encodes part of the MARK3 peptide identified in the CyTOF screen.

In order to experimentally verify the presence of this MARK3 splice isoform, the Ensembl database was checked to identify splice variants of MARK3 (FIG. 14b). This shows that there are a number of MARK3 splice isoforms present that incorporates Exon 24 (FIG. 14(b) and FIG. 14(c)). Primers (MARK3F: TCCCATGAAGCCACACCATTG (SEQ ID NO: 3) and MARK3R: AGCGTAGGGATCGAGGCITTG (SEQ ID NO: 4)) were designed in the flanking region to identify which MARK3 splice isoform is expressed.

The presence of the MARK3 splice-variant in GC cell lines was confirmed using RT-PCR (FIG. 14(c)). The PCR products were separated by size on a TBE-PAGE gel and visualized. Six out of the 16 GC cell lines (cell lines marked by an asterisk) expressed predominantly splice isoforms of the MARK3 gene that carried the peptide previously identified in the CyTOF screen, further demonstrating that it is a signature antigen shared by a GC patient subpopulation. Quantification of the expression of MARK3 isoforms in GC cell lines was performed using densitometry of the intensity of the PCR products is shown in FIG. 14(d). GC cell lines that express increased levels of MARK3 isoforms 1 and 3 include HFE 145, SNU1, GES1, HS738T, HS746T and HGC-27 (MARK3 isoforms land 3 in these cell lines comprise 71.0%, 68.7%, 30.1%, 96.1%, 44.7% and 49.1% of all isoforms, respectively). Majority of the other gastric cell lines express 10% or less of MARK3 isoforms 1 and 3. Furthermore, MARK3 isoform 1 and 3 expression was minimal or not seen in non-GC cell lines.

TABLE 1

| Constitutive Exons | Sequence |
|---|---|
| chr14:103958114- | Cactattcctgatcagagaactccagttgcttcaacacacagtatcagtagtgcag |
| 103958371 (Exon 23) | ccaccccagatcgaatccgcttcccaagaggcactgccagtcgtagcactttcca |
| Exon23 contains part of | cggccagccccgggaacggcgaaccgcaacatataatggccctcctgcctctcc |
| RNMSFRFIK peptide | cagcctgtcccatgaagccacaccattgtcccagactcgaagccgaggctccact |
| | aatctctttagtaaattaacttcaaaactcacaaggag |
| | (SEQ ID NO: 5) |
| chr14:103969219- | Tcgcaatgtatctgctgagcaaaaagatgaaaacaaagaagcaaagcctcgatc |
| 103970166 (Exon 26) | cctacgcttcacctggagcatgaaaaccactagttcaatggatcccggggacatg |
| | atgcgggaaatccgcaaagtgttggacgccaataactgcgactatgagcagagg |
| | gagcgcttcttgctcttctgcgtccacggagatgggcacgcggagaacctcgtgc |
| | agtgggaaatggaagtgtgcaagctgccaagactgtctctgaacggggtccggtt |
| | taagcggatatcggggacatccatagccttcaaaaatattgcttccaaaattgccaa |
| | tgagctaaagctgtaacccagtgattatgatgtaaattaagtagcaattaaagtgtttt |
| | cctgaacactgatggaaatgtatagaataatatttaggcaataacgtctgcatcttct |
| | aaatcatgaaattaaagtctgaggacgagagcacgcctgggagcgaaagctggc |
| | cttttttctacgaatgcactacattaaagatgtgcaacctatgcgccccctgccctact |
| | tccgttaccctgagagtcggtgtgtggccccatctccatgtgcctcccgtctgggtg |
| | ggtgtgagagtggacggtatgtgtgtgaagtggtgtatatggaagcatctccctac |
| | actggcagccagtcattactagtacctctgcgggagatcatccggtgctaaaacatt |
| | acagttgccaaggaggaaaatactgaatgactgctaagaattaaccttaagaccag |
| | ttcatagttaatacaggtttacagttcatgcctgtggttttgtgtttgttgttttgtgttttttt |
| | agtgcaaaaggtttaaatttatagttgtgaacattgcttgtgtgtgtttttctaagtagat |
| | tcacaagataattaaaaattcactttttctcagtaa |
| | (SEQ ID NO: 6) |
| Skipped Exons | Sequence |
| chr14:103964839- | aaacatgtcattcaggtttatcaaaag |
| 103964865 | (SEQ ID NO: 7) |
| Exon24- contains part of | |
| RNMSFRFIK peptide | |
| chr14:103966493- | Gcttccaactgaatatgagaggaacgggagatatgagggctcaag |
| 103966537 (Exon25) | (SEQ ID NO: 8) |

### Example 5

Use of Shared Splice Variant Antigen for Detection (Diagnosis) Purposes

A shared spliced variant antigen, such as MARK3, can be used for detection (diagnostic) purposes. In the case of MARK3, RNA from FFPE samples was extracted and converted to cDNA using gene specific primers (MARK3R, SEQ ID NO: 4). MARK3 splice variant was detected using RT-PCR (MARK3F (SEQ ID NO: 3) and MARK3R (SEQ ID NO: 4)). DNA gel electrophoresis was subsequently used to identify which MARK3 isoforms were present and also quantitate the percentage of MARK3 isoform 1 and 3 out of all MARK3 isoforms in these sample (FIG. 14(e)). The samples comprise of FFPE samples of GC tumours or bariatric stomach tissue from obese patients (non-cancerous). Quantification of MARK3 isoforms was performed using densitometry of the intensity of the PCR products. Bariatric stomach FFPE samples contain low levels of these isoforms, less than 5%, whereas 7 out of 20 of the GC FFPE samples contain greater than 10% of these isoforms (underlined samples in FIG. 14(e)).

Other shared spliced variant antigens (such as NBPF9, PARD3, ZC3HAV1, YAF2, CAMKK1, LRR1, ZNF670, GRINA or MZF1) can similarly be used for detection (diagnostic) purposes using the methods as described above.

### Example 6

Expansion of Shared Antigen-Directed T Cells

T cells having a shared splice variant antigen, such as MARK3, can be expanded ex-vivo. In the case of MARK3, PBMC were obtained from healthy donors. An aliquot of these PBMCs was used to isolate monocytes (Human Monocyte Isolation Kit, STEMCELL Technologies) for subsequent differentiation to dendritic cells (ImmunoCult Dendritic Cell Culture kit, STEMCELL Technologies). These monocyte derived dendritic cells were treated with mitomycin C (30 μg/ml) to prevent outgrowth of these cells. These dendritic cells were then loaded with MARK3 peptide and used as antigen-presenting cells. CD8+ T cells were isolated (EasySep Human CD8+ T cell isolation kit, STEMCELL Technologies) from another aliquot of PBMCs and these cells were co-cultured with the antigen-presenting cells to stimulate the expansion of MARK3 specific T cells. To generate sufficient number of MARK3-specific T cells for functional characterisation or TCR identification, MARK3 peptide loaded monocyte derived dendritic cells or artificial antigen presenting cells may be used to further stimulate expansion of MARK3 specific T cells. Expansion of MARK3 specific T cells using the above method can be used for the treatment of patients.

T cells having other shared splice variant antigens (such as NBPF9, PARD3, ZC3HAV1, YAF2, CAMKK1, LRR1, ZNF670, GRINA or MZF1) can similarly be isolated and expanded using the above methods.

### Example 7

Functional Significance of CD8+T Lymphocytes Responsive to the Shared MARK3 Splice Variant Antigen in GC

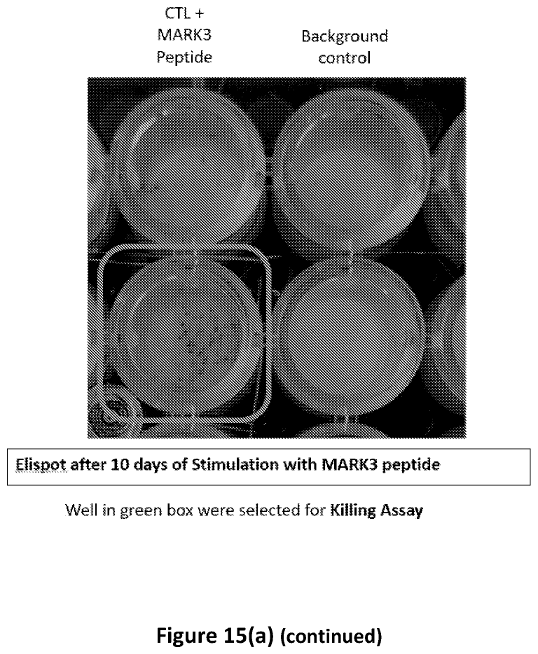

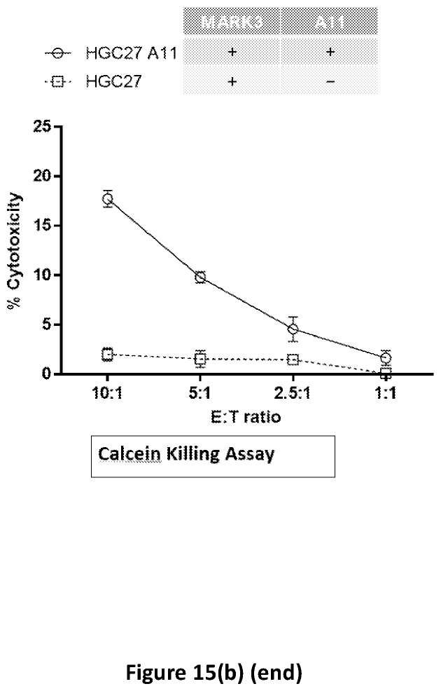

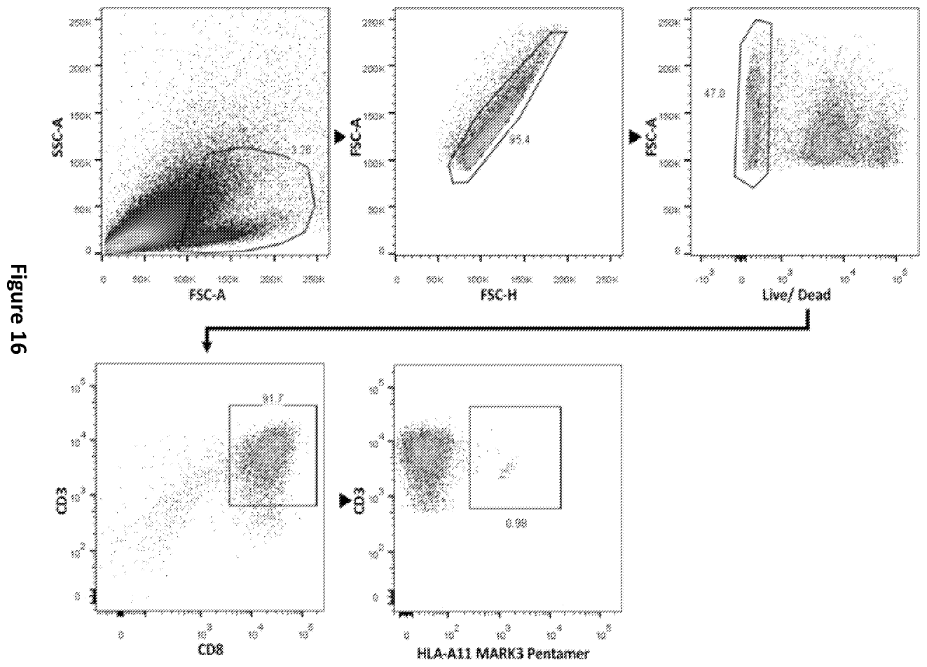

Characterization of the MARK3 antigen and CD8+T lymphocytes was carried out by first determining whether MARK3 specific CTLs could be expanded in healthy donor PBMCs (as shown in Example 6). FIG. 15(a) shows the results of an ELISPOT assay for IFN-γ in PBMCs from healthy donors with or without stimulation with MARK3 peptide. CTLs only secrete IFN-γ when they recognize their cognate antigen. From this Figure, IFN-γ-secreting CTLs were only observed when PBMCs were stimulated with MARK3 peptide. This shows that the MARK3 splice variant antigen is antigenic and can stimulate the expansion of MARK3-specific CTLs.

To show that tumour cells express the MARK3 antigen and that MARK3-specific CTLs can target these tumour cells, the MARK3-specific CTLs were used to test their ability to kill GC cell lines (HGC-27, identified in Example 4) that express the MARK3 splice variant antigen (49.1% of MARK3 isoforms comprise isoform 1 and 3, FIG. 14(d)). The original HGC-27 cell line (Riken cell bank: RCB0500) do not express HLA-A11 but a stable HCG-27 cell line expressing HLA-A11 was generated using lentiviral transduction. Only tumour cells carrying both the HLA-A11 allele and the MARK3-SV mRNA transcript were killed by MARK3-SVP stimulated CTLs. In contrast, tumour cells expressing the MARK3-SV mRNA transcript but not the HLA-A11 MHC allele were not killed by the MARK3-SVP stimulated CTLs (FIG. 15(b)).

### Example 8

Isolation of MARK3 Shared Antigen-Directed T Cells

To isolate MARK3-specific T cells, the CD8+ T cells that were co-cultured with MARK3 peptide loaded dendritic cells (as shown in Example 6) were first stained with MARK3 pentamer-PE (custom pentamer, PROIMMUNE), followed by anti-CD3-FITC and anti-CD8a-APC. BD. FIG. 16 shows the gating strategy of the cell sorting procedure for isolating MARK3-specific CD8+ T cells using the FACSAria III. Single cells were sorted into a PCR plate containing 1.5 μl lysis buffer (5 units RNase inhibitor, 0.2% Triton X-100, 0.5 mM dNTP mix, 0.1 μM TRAC primer: GACCAGCTTGACATCACAG (SEQ ID NO: 9), and 0.1 μM TRBC primer: CTCAGGCAGTATCTGGAGTCATTG (SEQ ID NO: 10)). cDNA was subsequently prepared by reverse transcription in a 2.5 μl reaction (SuperScript III Reverse Transcriptase, Thermo Fisher Scientific) using the lysates from the single cell sorted MARK3-specific T cells.

### Example 9

Amplification and Sequencing of MARK3-Specific TCR (TRA and TRB Chains)

TRA and TRB chain sequences were obtained by nested PCRs (PCR1 and PCR2). The TRAV and TRBV primers used for the two rounds of PCRs are described in Wang et al (2012) Sci. Transl Med (Table S1, External primers used for PCR1 and Internal primers used for PCR2). The TRAC and TRBC primer used for the two PCRs are:

TRAC PCR1:
(SEQ ID NO: 11)
TGCTGTTGTTGAAGGCGTTTG;
TRAC PCR2:
(SEQ ID NO: 12)
TGTTGCTCTTGAAGTCCATAG;
TRBC PCR1:
(SEQ ID NO: 13)
CCCACTGTGCACCTCCTTC;
and
TRBC PCR2:
(SEQ ID NO: 14)
TTCTGATGGCTCAAACACAG.

The first PCR was done by using cDNA prepared from single-cell sorted MARK3-specific T cells and combining external primers for TRAV and TRBV (0.1 μM each), TRAC PCR1 (0.4 μM) and TRBC PCR1 (0.4 μM). PCR1 was then used as the template for the second PCR in two separate PCR reactions to generate TRA and TRB PCR products. The internal primers for TRAV (0.1 μM each) and TRAC PCR2 (0.4 μM) were used to obtain TRA Sequences, whereas internal primers for TRBV (0.1 μM each) and TRBC PCR2 (0.4 μM) were used to obtain TRB sequences.

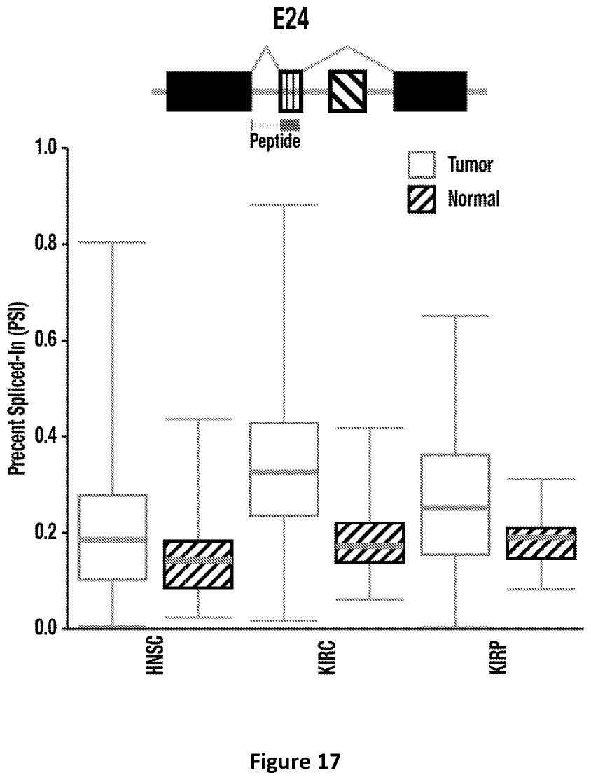

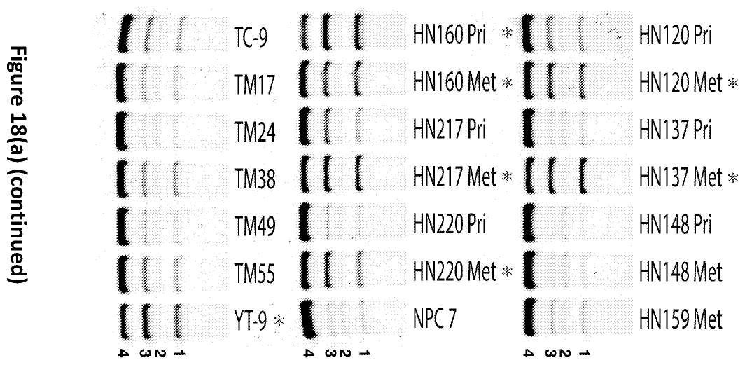

PCR products for TRA and TRB after the second PCR were analysed by gel electrophoresis to identify clones which had successful amplification of both TRA and TRB (FIG. 17). These TRA and TRB PCR products were then sequenced using Sanger sequencing (BigDye Terminator v3.1, Thermo Fisher Scientific) using TRAC PCR2 and TRBC PCR2 primer, respectively.

TRA and TRB sequences were analysed using the IMGT database to identify the V, J and CDR3 regions. From this analysis, in one clone a TRA sequence consisting of TRAV6*03, TRAJ9*01 and CDR3 comprising the amino acids “CAPYTGGFKTIF” (SEQ ID NO: 20), and a TRB sequence consisting of TRBV7-9*01, TRBJ1-2*01 and CDR3 comprising the amino acids “CASSSPRVGYGYTF” (SEQ ID NO: 28), was identified (see below).

MARK3 TRA Chain
TRAV6*01 AA sequence:
(SEQ ID NO: 15)
MESFLGGVLLILWLQVDWVKSQKIEQNSEALNIQEGKTATLTCNYTNYSPA
YLQWYRQDPGRGPVFLLLIRENEKEKRKERLKVTFDTTLKQSLFHITASQP
ADSATYL
TRAJ9*01 AA sequence:
(SEQ ID NO: 16)
GAGTRLFVKAN
CDR1 AA sequence
(SEQ ID NO: 17)
NYSPAY
CDR2 AA sequence
(SEQ ID NO: 18)
IRENEKE
CDR3 nucleotide sequence:
(SEQ ID NO: 19)
TGTGCTCCGTATACTGGAGGCTTCAAAACTATCTTT
CDR3 AA sequence:
(SEQ ID NO: 20)
CAPYTGGFKTIF
MARK3 TRA variable region AA sequence
(SEQ ID NO: 21)
MESFLGGVLLILWLQVDWVKSQKIEQNSEALNIQEGKTATLTCNYTNYSPA
YLQWYRQDPGRGPVFLLLIRENEKEKRKERLKVTFDTTLKQSLFHITASQP
ADSATYLCAPYTGGFKTIFGAGTRLFVKAN
MARK3 TRA AA sequence
(SEQ ID NO: 22)
MESFLGGVLLILWLQVDWVKSQKIEQNSEALNIQEGKTATLTCNYTNYSPA
YLQWYRQDPGRGPVFLLLIRENEKEKRKERLKVTFDTTLKQSLFHITASQP
ADSATYLCAPYTGGFKTIFGAGTRLFVKANIQNPDPAVYQLRDSKSSDKSV
CLFTDFDSQTNVSQSKDSDVYITDKTVLDMRSMDFKSNSAVAWSNKSDFAC
ANAFNNSIIPEDTFFPSPESSCDVKLVEKSFETDTNLNFQNLSVIGFRILL
LKVAGFNLLMTLRLWSS
MARK3 TRB Chain
TRBV7-9*01 AA sequence:
(SEQ ID NO: 23)
MGTSLLCWMALCLLGADHADTGVSQNPRHKITKRGQNVTFRCDPISEHNRL
YWYRQTLGQGPEFLTYFQNEAQLEKSRLLSDRFSAERPKGSFSTLEIQRTE
QGDSAMYL
TRAJ1-2*01 AA sequence:
(SEQ ID NO: 24)
GSGTRLTVV
CDR1 AA sequence
(SEQ ID NO: 25)
SEHNR
CDR2 AA sequence
(SEQ ID NO: 26)
FQNEA
CDR3 nucleotide sequence:
(SEQ ID NO: 27)
TGTGCCAGCAGCTCCCCCCGGGTTGGCTATGGCTACACCTTC
CDR3 AA sequence:
(SEQ ID NO: 28)
CASSSPRVGYGYTF
MARK3 TRB variable region AA sequence
(SEQ ID NO: 29)
MGTSLLCWMALCLLGADHADTGVSQNPRHKITKRGQNVTFRCDPISEHNRL
YWYRQTLGQGPEFLTYFQNEAQLEKSRLLSDRFSAERPKGSFSTLEIQRTE
QGDSAMYLCASSSPRVGYGYTFGSGTRLTVV
MARK3 TRB AA sequence
(SEQ ID NO: 30)
MGTSLLCWMALCLLGADHADTGVSQNPRHKITKRGQNVTFRCDPISEHNRL
YWYRQTLGQGPEFLTYFQNEAQLEKSRLLSDRFSAERPKGSFSTLEIQRTE
QGDSAMYLCASSSPRVGYGYTFGSGTRLTVVEDLNKVFPPEVAVFEPSEAE
ISHTQKATLVCLATGFFPDHVELSWWVNGKEVHSGVSTDPQPLKEQPALND
SRYCLSSRLRVSATFWQNPRNHFRCQVQFYGLSENDEWTQDRAKPVTQIVS
AEAWGRADCGFTSVSYQQGVLSATILYEILLGKATLYAVLVSALVLMAMVK
RKDSRG

### Example 10

Detecting the MARK3 Splice Variant-Specific T Lymphocytes for the Treatment of Cancer Patients within the Population Who Express the MARK3 Splice Variant

HLA-A11 tetramers loaded with the MARK3 peptide were used to determine whether GC patients had MARK3-specific T lymphocytes. The histogram in FIG. 12(a) shows that one out of 7 GC patients (patient SC020) had T lymphocytes that recognized the MARK3 SVA. These MARK3-specific T lymphocytes in patient SC020 can be further expanded for the treatment of gastric cancers which carry the characteristic MARK3 SVA. Expansion of MARK3-specific T lymphocytes that are present in other MARK3-splice variant expression-positive patients may be carried out as described in Example 6.

### Example 11

Identification of MARK3 Splice Event in HNSC

The presence of MARK3 SVA was verified in other cancer types after identification in GC. This was done by cross-referencing publicly available databases that analysed alternative splicing such as TCGA SpliceSeq: (https://bioinformatics.mdanderson.org/TCGASpliceSeq/singlegene.jsp).

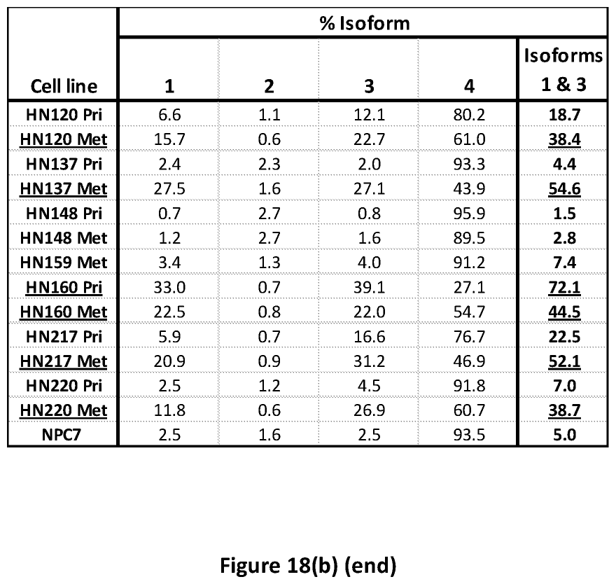

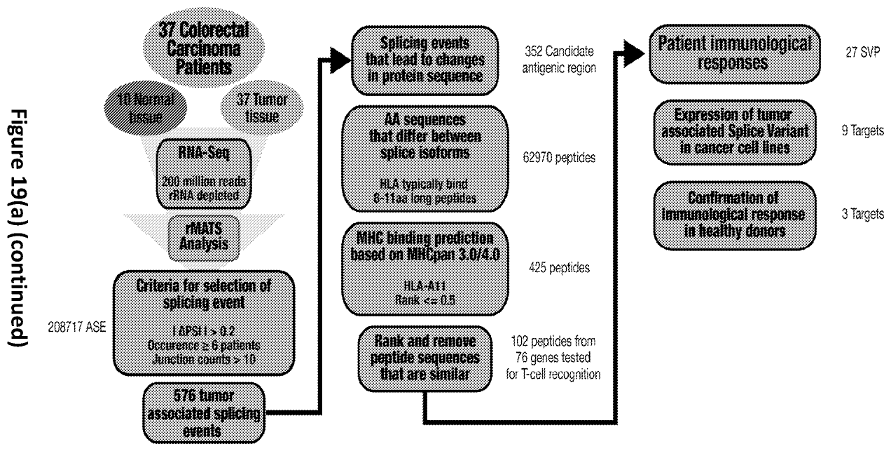

Through this analysis MARK3 was found to be also aberrantly spliced in HNSC and KIRC (FIG. 17). The presence of MARK3 splice-variant transcript expression in cell lines derived from HNSC patients was confirmed using RT-PCR (FIG. 18) using primers described in Example 4. FIG. 18(a) shows that 7 out of 21 HNSC cell lines (indicated by asterisks) expressed predominantly the alternatively spliced isoforms identified in the first GC patient cohort, demonstrating that the corresponding MARK3 antigenic peptide is also a potential shared antigen in HNSC. Quantification of the MARK3 isoforms is shown in FIG. 18b.

### Example 12

Identification of Alternative Spliced Variants and Prediction of HLA-A11 Binding Peptides in Colorectal Carcinoma

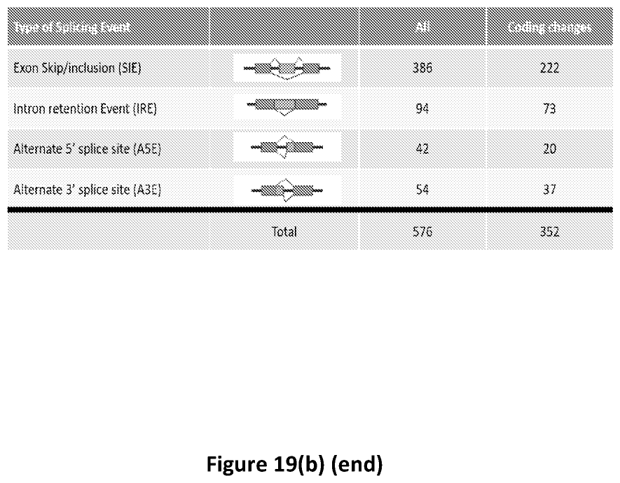

Colorectal carcinoma (CRC) tumour samples (37) and matched normal samples (10) were taken from non-malignant tissue adjacent to tumour and these samples constituted the discovery cohort. These samples along with their clinical information were obtained from Singapore Health Services tissue repositories. Deep RNA sequencing (100 million Paired-End reads) and mRNA splicing analysis using rMATS were performed on these samples to identify tumour-associated alternative splicing events. Selection criteria (at least 20% change in splicing (ΔPSI), occurrence in at least 6 patients, and junction counts for inclusion/skipping must be >10) were applied, which yielded a list of 576 tumour-associated alternative splice events of which 352 leads to changes in protein sequence (FIG. 19(a) and FIG. 19(b)).

These tumour-associated alternative splicing events were used for the identification of candidate antigenic regions which may be shared in a CRC patient subpopulations, or subgroups, by looking for HLA-binding peptides. NetMHCpan 3 and 4 were used for predicting 8-11 amino acid-long peptides that could bind to HLA-A11, an HLA allele that is present in approximately 50% of the CRC patient cohorts. Both versions of NetMHCpan were used to be more inclusive of the peptides that were used for screening. Shared peptide antigens (102) were selected based on a selection criterion (Rank <=0.5 in either NetMHCPan 3 or NetMHCPan 4) and then peptides that were similar were removed. In the subsequent HLA-A11 CyTOF screen, these 102 peptide antigens corresponded to 76 splice events. FIG. 19(c) summarizes the ΔPSI and the co-occurrence of the splicing alterations that gave rise to the 102 peptide sequences which define the CRC splice variant antigen sub-groups.

### Example 13

Validation of Shared HLA-A11 Splice Variant Antigens and Identification of Splice Variant-Specific CD8+ T Cells in CRC Patients

To determine if CRC patients have CD8+ T cells that target any of the 102 peptides that were identified in Example 12, a CyTOF screen was conducted on a new cohort of 8 CRC patients (validation cohort) using MHC tetramer staining of PBMC (FIG. 19(a)). These CRC patients are all positive for HLA-A11.

The 102 peptides were chemically synthesized by Mimotopes. These peptides were loaded onto biotinylated HLA-A11 by UV-mediated exchange. Streptavidin, labelled with three heavy metal barcodes, was bound to the peptide-loaded HLA-A11 to make the HLA-tetramers used for staining PBMCs.

To increase the sensitivity of detecting antigen-specific T cells, two different sets of heavy metal barcodes were used for each peptide. PBMCs from each patient were stained with these two sets of peptide-loaded HLA tetramers. Frequency co-concordance was used as an indication of specific staining for rare events.

In total we identified antigen specific CD8+ T cells that target 27 splice variant peptides (FIG. 19(a)). Eight of these SVAs are shown in FIG. 20(a) and could be identified in one or more CRC patients. These SVAs were derived from aberrant splicing of NBPF9, PARD3, ZC3HAV1, YAF2, CAMKK1, LRR1, ZNF670 and GRINA with the following peptide sequences: SSFYALEEK (SEQ ID NO: 31), SQLDFVKTRK (SEQ ID NO: 32), LTMAVKAEK (SEQ ID NO: 33), VIVSASRTK (SEQ ID NO: 34), VTSPSRRSK (SEQ ID NO: 35), SLPRFGYRK (SEQ ID NO: 36), SCVSPSSELK (SEQ ID NO: 37), and SIRQAFIRK (SEQ ID NO: 38). Two of these SVAs, NBPF9 and ZNF670, could be detected in two patients, further indicating that these SVAs are shared and immunogenic across patients. The occurrence, ΔPSI and type of splice event that gave rise to these SVAs are shown in FIG. 20(a). All four of these SVAs were detected in two different cohorts of CRC patients (discovery and validation cohort).

The coordinates of the exons or introns that are aberrantly spliced and tumour-associated isoforms are shown in FIG. 20(b). For the splice event that gave rise to the NBPF9 SVA, it is an intron retention event that results in the retention of an intron (chr1:144826287:144826932:+). This results in transcripts that contains the intron (chr1:144826235:144827105:+). For the splice event that gave rise to the PARD3 SVA, it is an alternative usage of 5′ splice site that results in transcripts that contain the exons (chr10:34625127:34625171:− and chr10:34626206:34626354:−). For the splice event that gave rise to the ZC3HAV1 SVA, it is also an alternative usage of 5′ splice site that results in transcripts that contain the exons (chr7:138763298:138763399:−, and chr7:138763850:138764989:−). For the splice event that gave rise to the YAF2 SVA, it is an alternative usage of 3′ splice site that results in transcripts that contain the exons (chr12:42604350:42604421:−, and chr12:42631401:42631526:−). For the splice event that gave rise to the CAMKK1 SVA, it is an exon skip/inclusion event that results in the skipping of an exon (chr17:3784921-3784942:−(SEQ ID NO: 39)). This results in transcripts that contain the exons (chr17:3785822-3785858:− and chr17:3783640-3783728:−). For the splice event that gave rise to the LRR1 SVA, it is an exon skip/inclusion event that results in the skipping of an exon (chr14:50074118-50074839:+(SEQ ID NO: 42)). This results in transcripts that contain the exons (chr14:50069088-50069186:+ and chr14:50080974-50081389:+). For the splice event that gave rise to the ZNF670 SVA, it is an exon skip/inclusion event that results in the skipping of an exon (chr1:247130997-247131094:−(SEQ ID NO: 45)). This results in transcripts that contains the exon (chr1:247151423-247151557:− and chr1:247108849-247109129:−). For the splice event that gave rise to the GRINA SVA, it is an intron retention event that results in the removal of an intron (chr8:145065973: 145066412:+). This results in transcripts that does not contain the intron (chr8:145065860-145065972:+@chr8:145066413-145066541:+). All of the coordinates described above are based on GRCh37/hg19 genome assembly.

### Example 14

Identification and Validation of Shared Candidate Antigens and their Cognate T Cells in Colorectal Cancer

FIG. 19a shows the workflow for identifying and validating shared candidate antigens and their cognate antigen specific T-cells in colorectal cancer. Shared candidate antigens from aberrant splicing that produced HLA-A11 binding peptides were identified as described in Example 12. An immunological screen using these HLA-A11 binding peptides was used to determine whether CRC patients had any immunological response to these candidate antigens (as shown in Example 13). From this immunological screen, it was found that CRC patients had antigen-specific T-cells against 27 splice variant peptides. The expression of the splice variant that gave rise to the splice variant peptides was confirmed for 9 of these targets by performing RT-PCR in colorectal cancer cell lines. It was also found that some cancer cell lines have increased expression of the tumour-associated splice variant compared to normal tissue for these 9 targets. Additionally, four of these targets show increased expression of the tumour-associated splice variant in tumour tissue samples compared to adjacent normal tissue samples from CRC patients. In-vitro experiments using CD8+ T cells from healthy donors were used to further test the immunogenicity of these targets and it was found that antigen-specific T cells could be generated for 3 of these targets. Accordingly, this approach allows rapid and simultaneous identification of shared candidate antigens as well as their cognate T cells, allowing the rapid development of T cell treatment option.

### Example 15

Identified Tumour-Associated Splice Variants are Present in Multiple Molecular Subtypes of CRC Patients

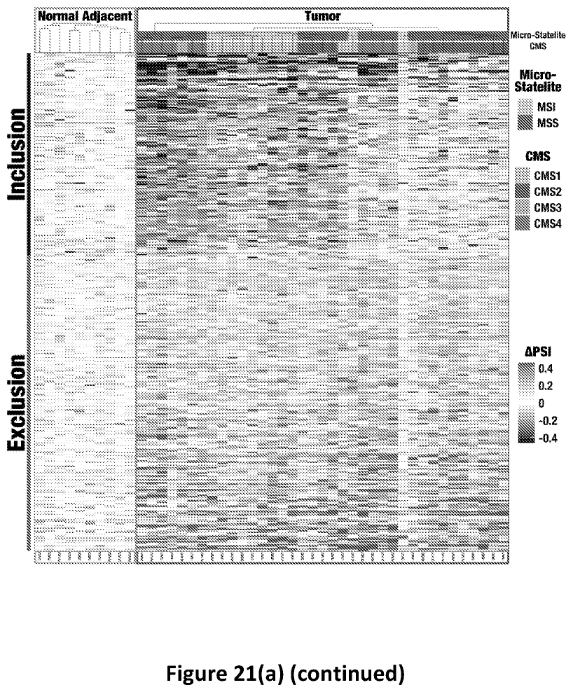

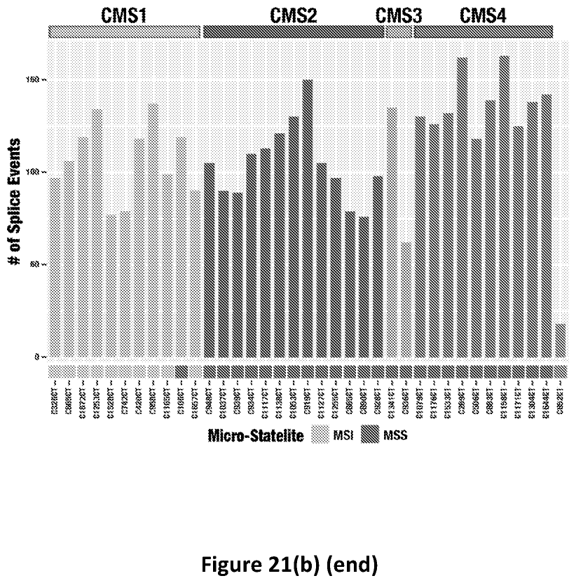

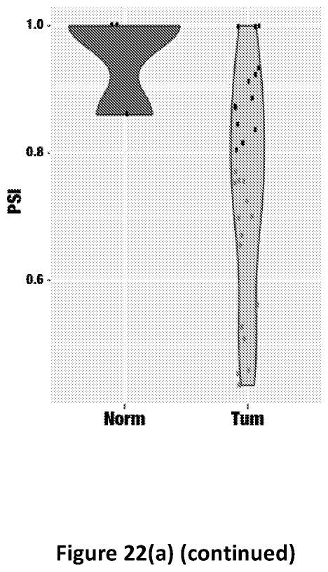

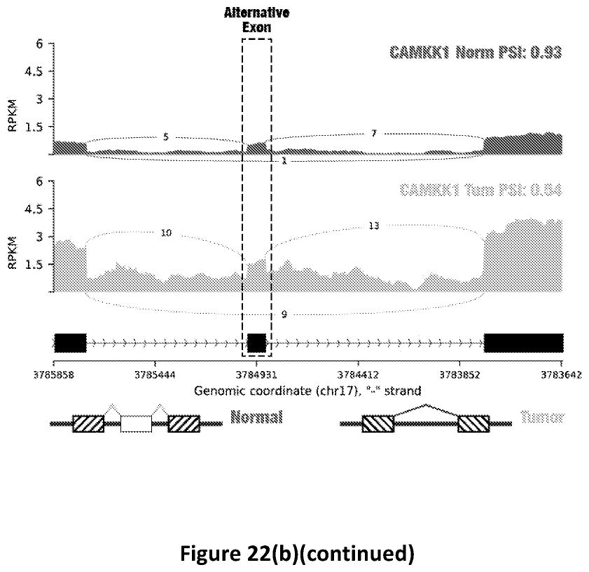

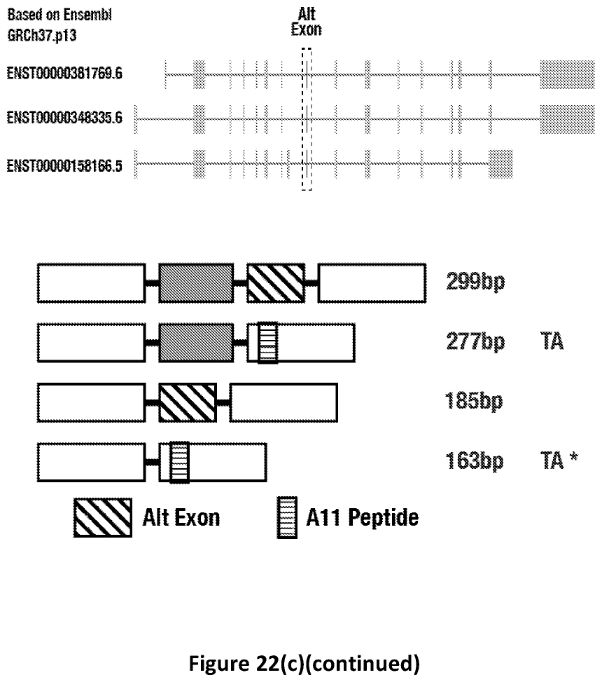

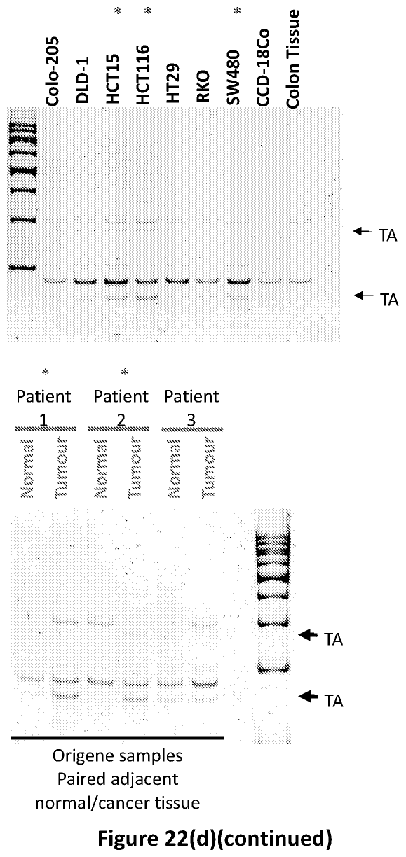

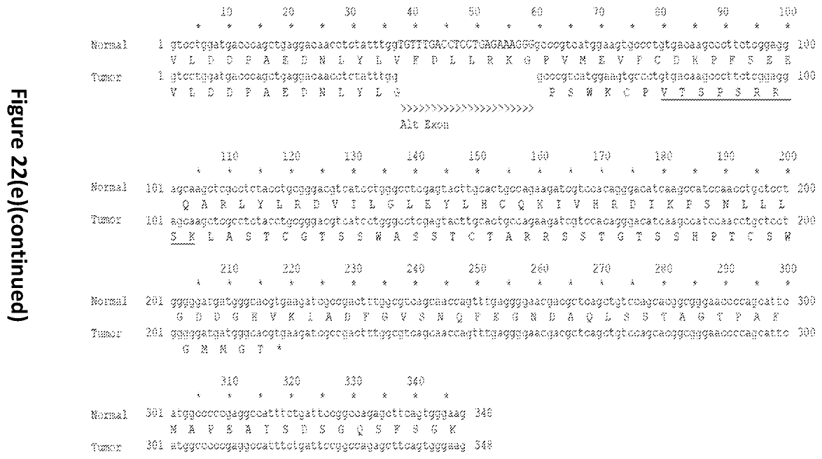

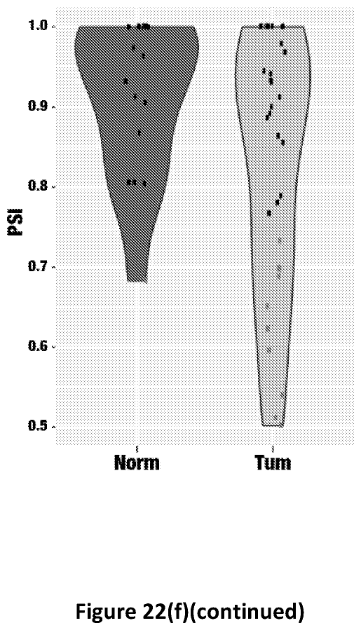

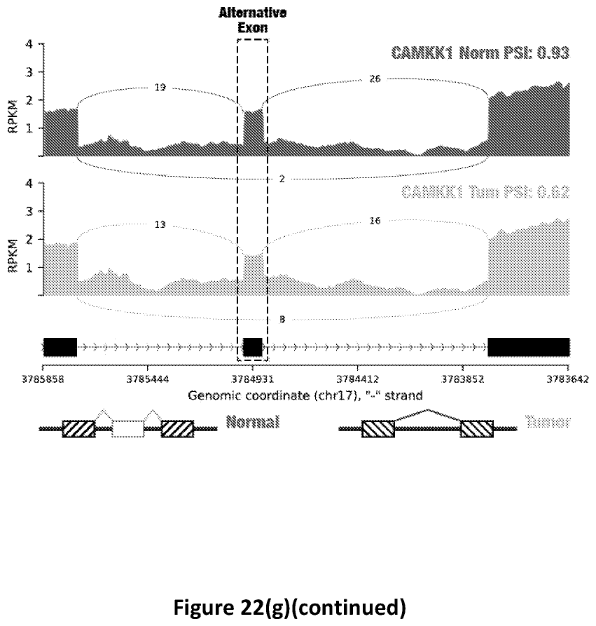

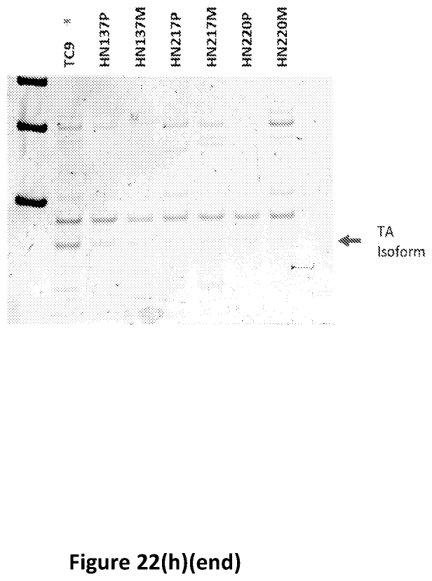

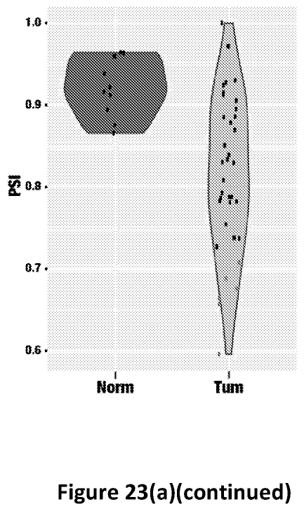

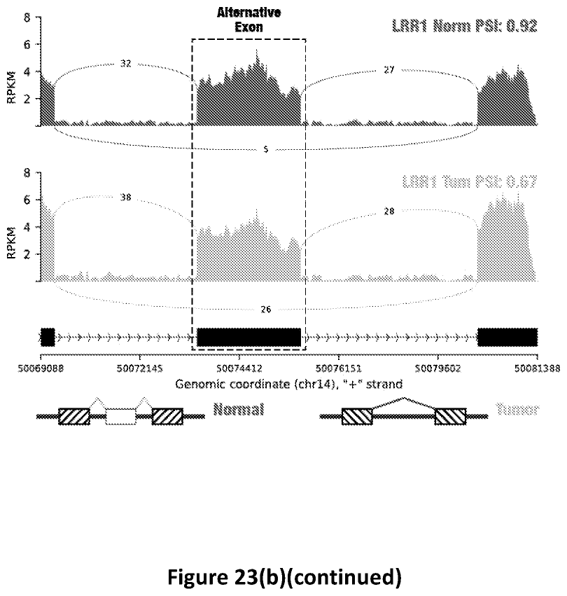

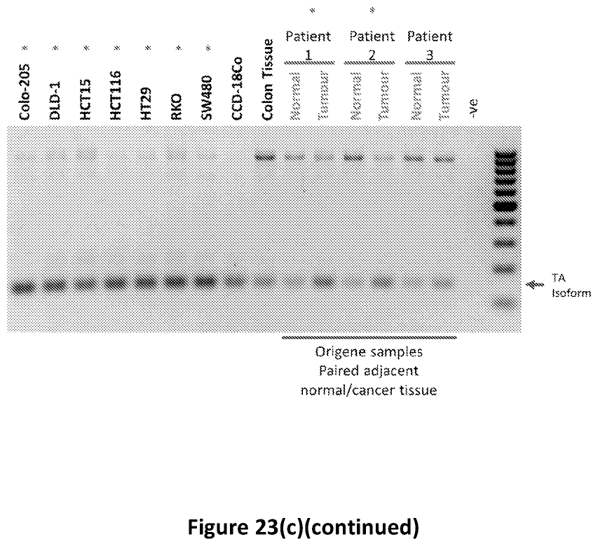

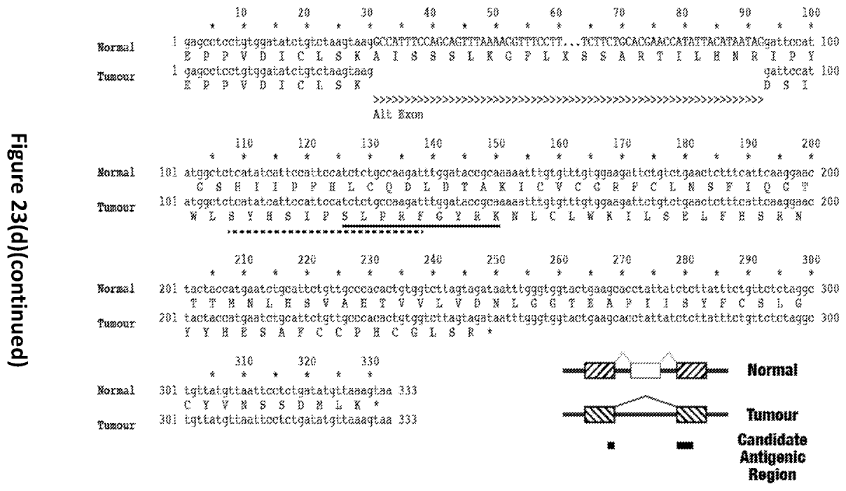

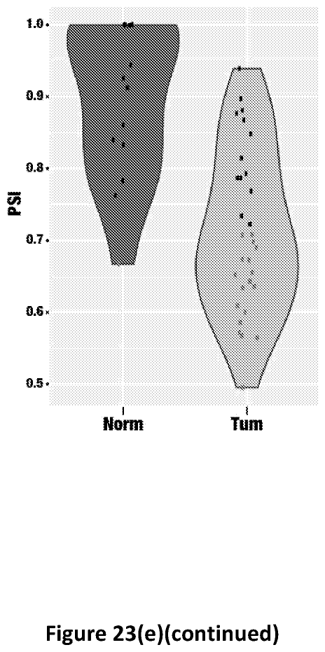

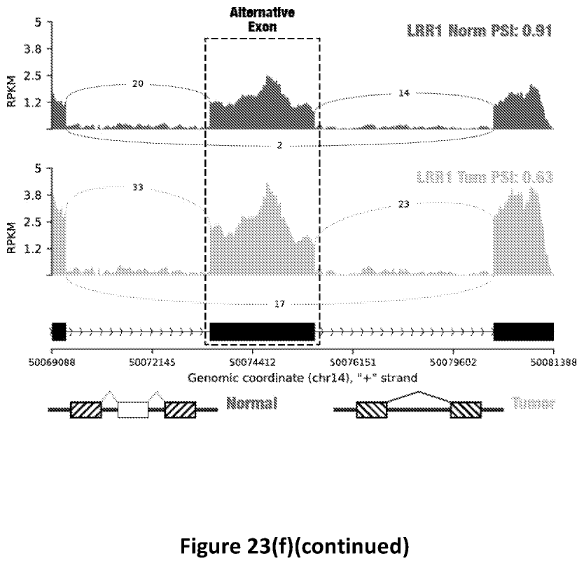

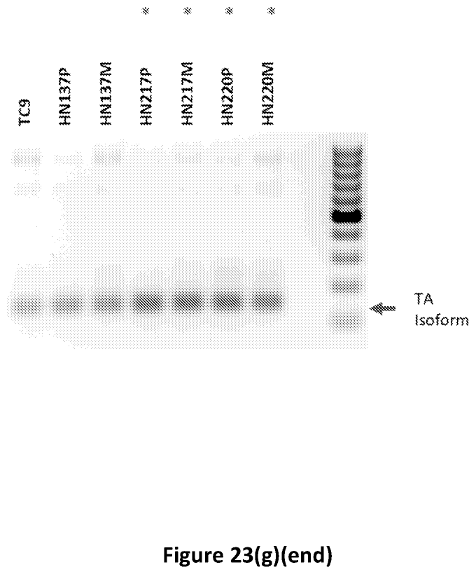

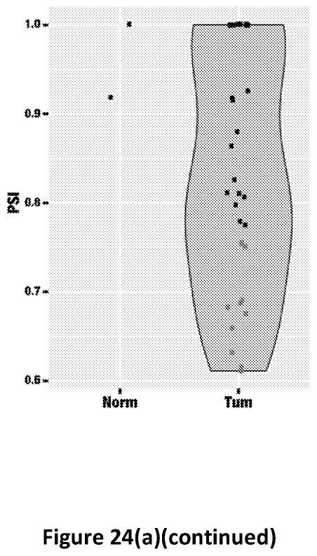

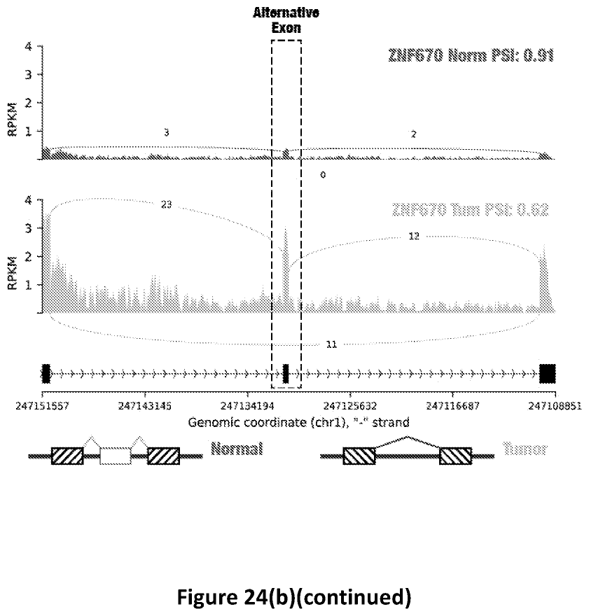

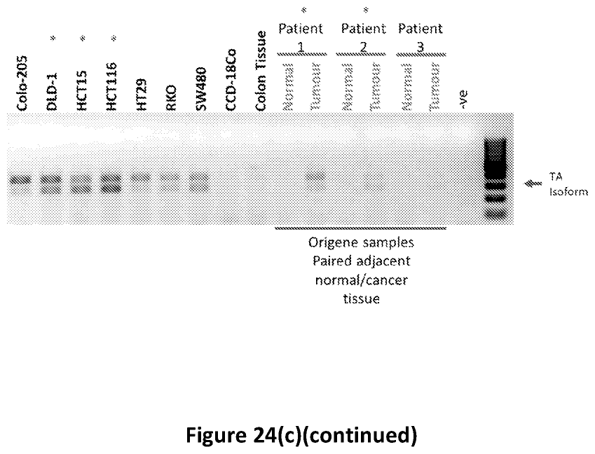

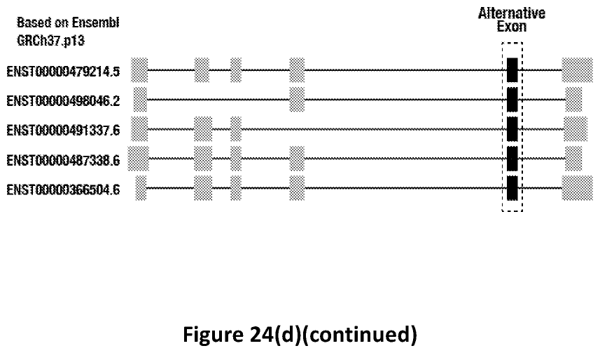

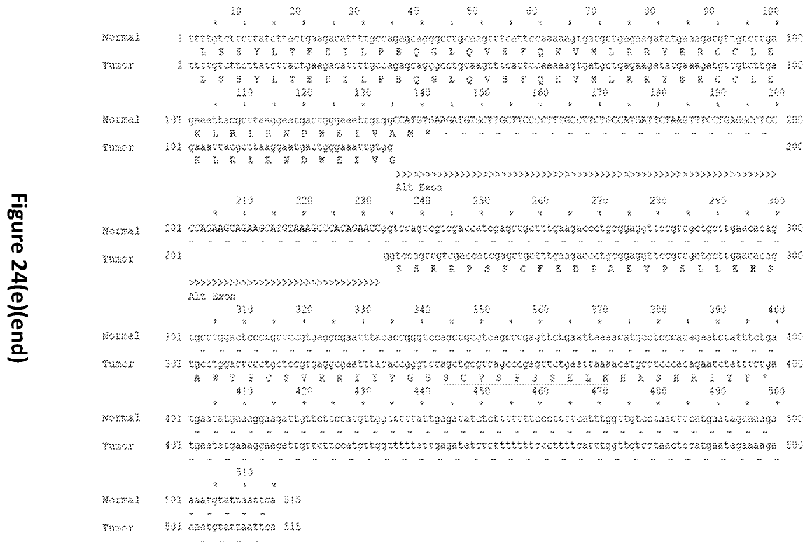

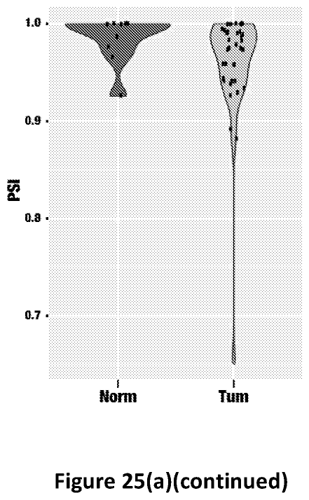

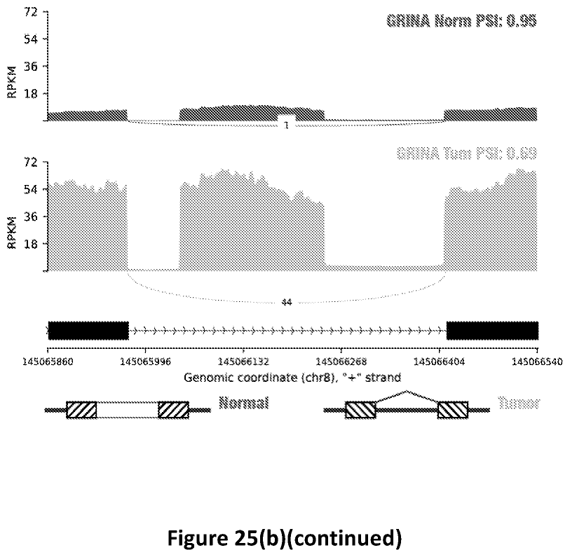

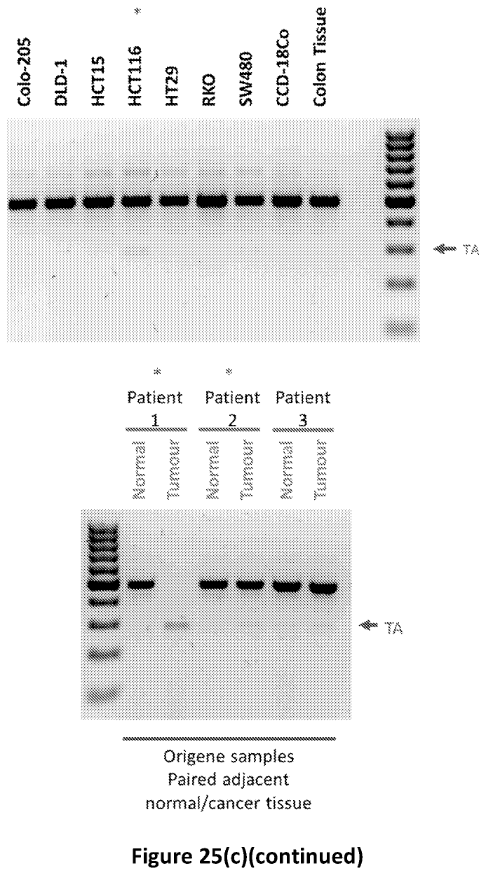

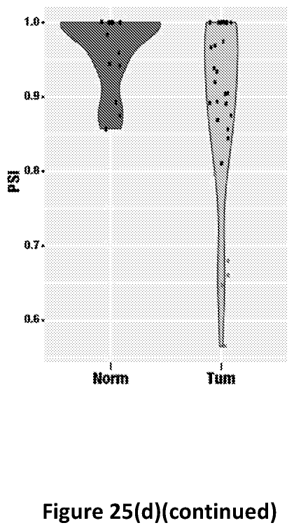

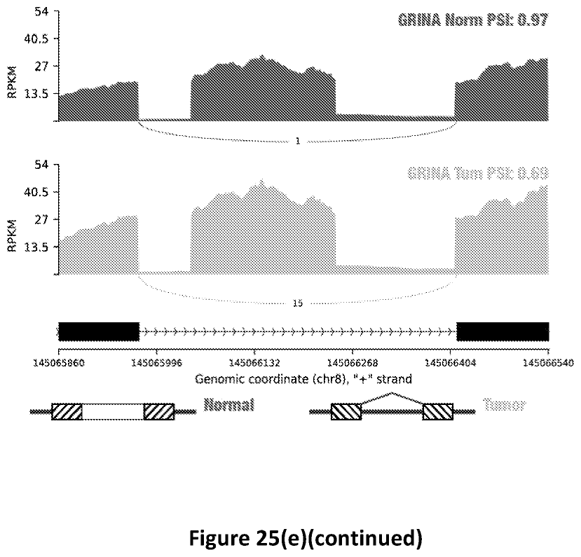

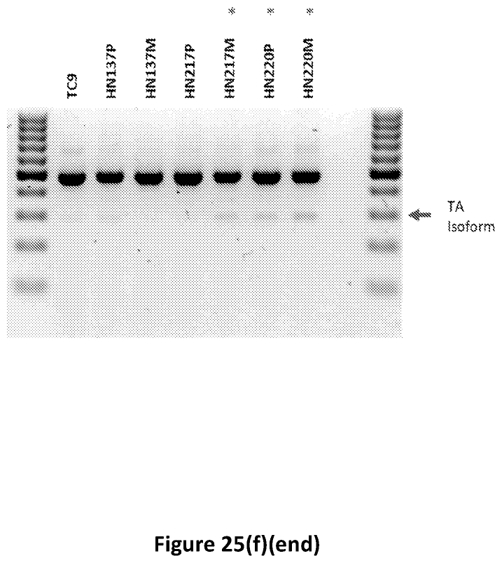

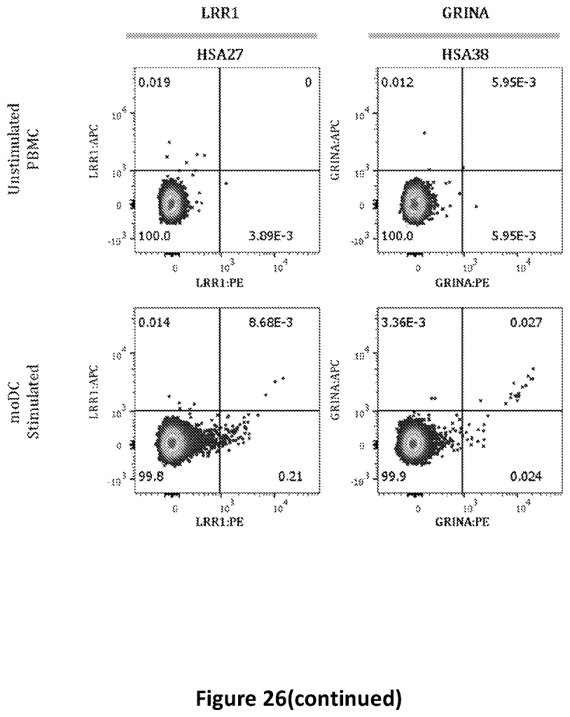

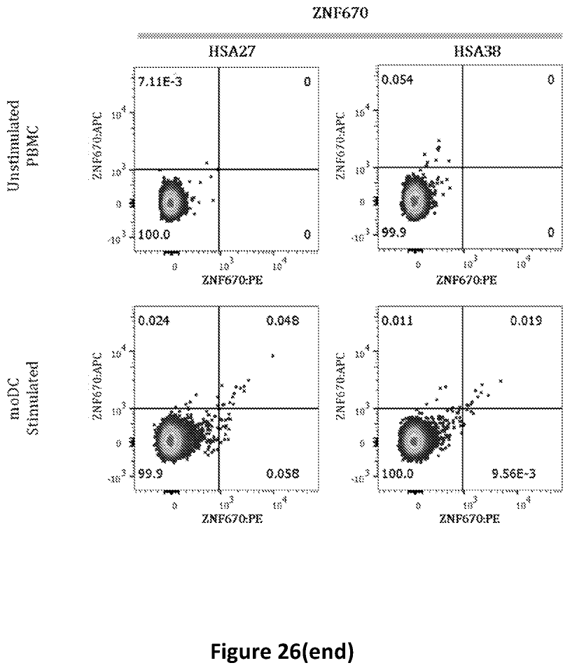

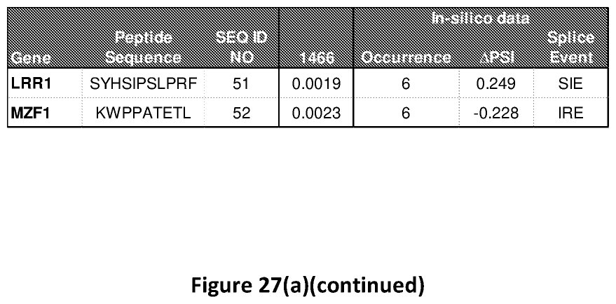

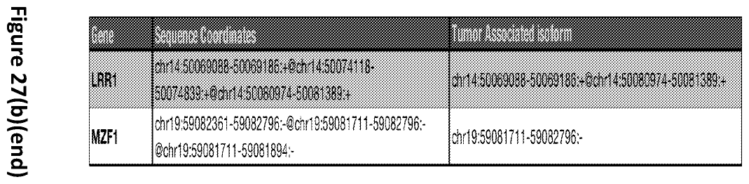

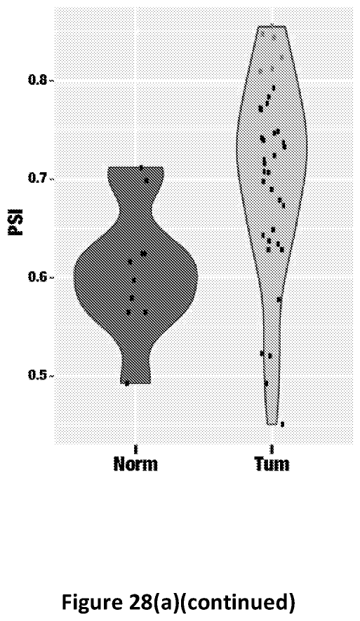

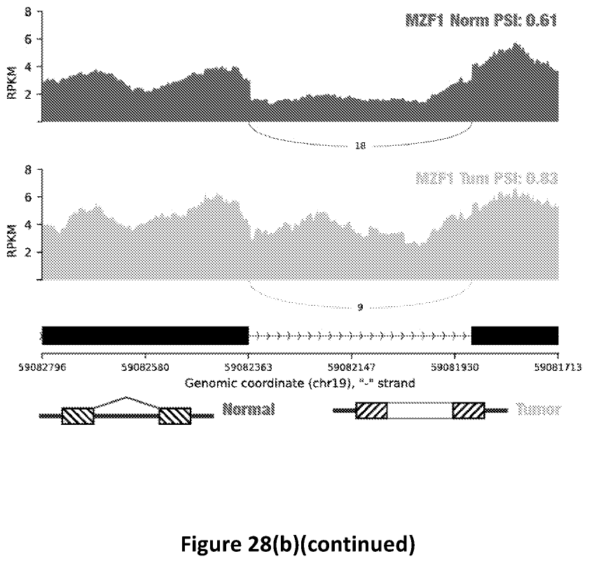

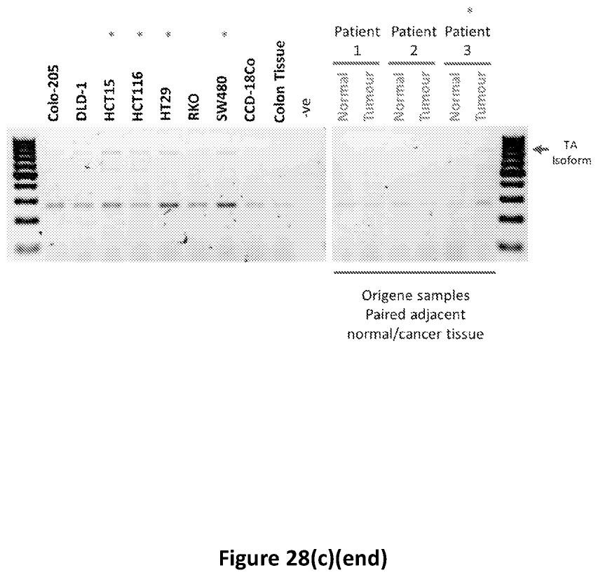

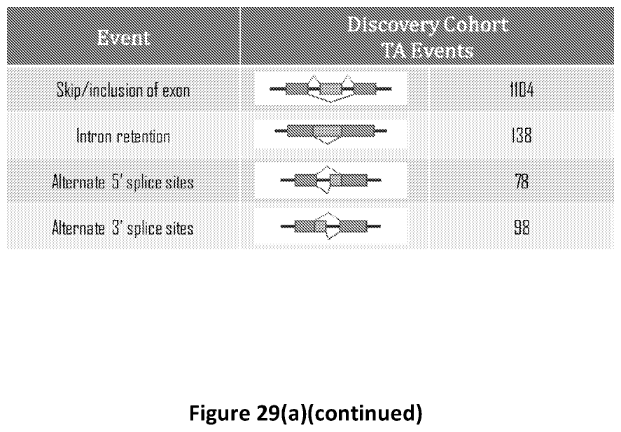

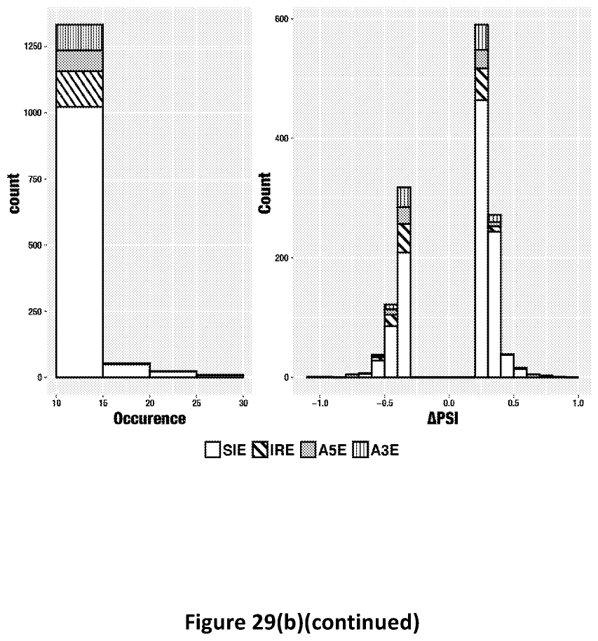

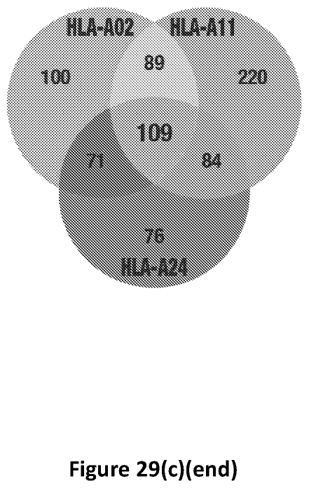

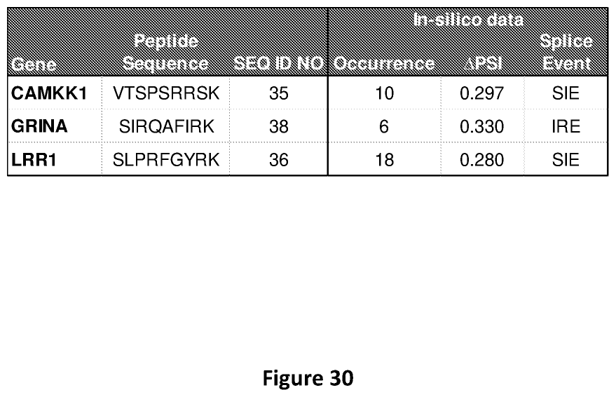

Tumour-associated splice variants identified as shown in Example 12 are present in multiple patients as shown in FIG. 21(a). These tumour-associated splice variants cause changes in protein sequence through either simple addition or omission of amino acids or by generating new protein sequences through changes in protein reading frame (FIG. 5). Neoantigens derived from somatic point mutations are found mainly in microsatellite instable (MSI) CRC patients (predominantly consensus molecular subtype (CMS) 1). In contrast, for the tumour-associated splice variants of the present invention (in addition to being present in multiple CRC patients), individual patients have similar numbers of tumour-associated splice variants present, regardless of either their microsatellite or CMS status (FIG. 21(b)).

### Example 16

Validation of CAMKK1 Splice Variant Antigen in CRC

A CAMKK1 splice variant peptide that binds to HLA-A11 (SEQ ID NO: 35) was identified as shown in Examples 12 and 13. Aberrant splicing of CAMKK1 was observed in 9 out of 37 CRC patients and median change in PSI was 0.473 between tumour and normal samples in the discovery cohort (FIG. 20(a)).

The PSI values for individual normal (Norm) and tumour (Turn) samples from CRC patients are shown in FIG. 22(a). Only samples which have sufficient junction counts are shown in this figure. FIG. 22(b) shows the sashimi plot for normal and tumour outlier samples (as was also described in Example 1); each sashimi plot shows the average read density of these samples. The sashimi plot for tumour samples shows that there is increased skipping of an exon. The tumour-associated splice event for CAMKK1 that was identified consists of skipping of an exon (chr17:3784921-3784942:−, SEQ ID NO: 39).

In order to experimentally verify the presence of the CAMKK1 splice isoform in CRC, RT-PCR was performed on cell lines and in tissue samples (matched tumour and adjacent normal samples) from three CRC patients (FIG. 22(c)). Primers (CAMKK1F: GAAGCTGGACCACGTGAATGTG (SEQ ID NO: 40) and CAMKK1R: AGTACTCGAGGCCCAGGATGAC (SEQ ID NO: 41)) were designed in the flanking region to identify which CAMKK1 splice isoform is expressed. These primers bind to sequences that flank the alternatively-spliced exon that was identified. Based on the GRCh37/hg19 genome assembly, there is another exon that is also alternatively spliced. Beside these two splice isoforms identified here, two additional splice isoforms could be created from the differential splicing of these two alternatively-spliced exons (FIG. 22(c)). The sizes of the PCR products for the tumour-associated splice variants are 277 bp and 163 bp.

It was confirmed that the 163 bp CAMKK1 tumour-associated splice variant was more highly expressed in a number of CRC cell lines (HCT15, HCT116 and SW480) compared to normal colon tissue (FIG. 22(d)). Using RNA that was isolated from matched adjacent normal and tumour tissue from three different CRC patients, cDNA was prepared and used for RT-PCR to detect CAMKK1 splice isoforms. Two of these CRC patients show increased expression of the CAMKK1 tumour-associated splice isoform (FIG. 22(d)). As was described in Example 5, this RT-PCR can be used for detection or diagnostic purposes.

The CAMKK1 tumour-associated splice variant that was identified involves the skipping of an exon with 22 nucleotides (SEQ ID NO: 39). Alternative splicing of this exon has not been observed before based on current gene annotation (Gencode version 34 or RefSeq) and represents a novel splice isoform. As shown in FIG. 22(c), skipping of this exon causes a change in reading frame for the downstream exon, which leads to formation of the HLA-A11 binding peptide that was identified in FIG. 20(a).

### Example 17

Validation of LRR1 Splice Variant Antigen in CRC

A LRR1 splice variant peptide that binds to HLA-A11 (SEQ ID NO: 36) was identified as described in Examples 12 and 13. Aberrant splicing of LRR1 was observed in 6 out of 37 CRC patients and median change in PSI was 0.249 between tumour and normal samples in the discovery cohort (FIG. 20(a)).

The PSI values for individual normal and tumour samples from CRC patients are shown in FIG. 23(a). Only samples which have sufficient junction counts are shown in this figure. FIG. 23(b) shows the sashimi plot for normal and tumour outlier samples (as was described in Example 1), each sashimi plot shows the average read density of these samples. The sashimi plot for tumour samples shows that there is increased skipping of an exon. The tumour-associated splice event for LRR1 that was identified consists of skipping of an exon (chr14:50074118-50074839:+, SEQ ID NO: 42).

In order to experimentally verify the presence of the LRR1 splice isoform in CRC, RT-PCR was performed on cell lines and in tissue samples (matched tumour and adjacent normal samples) from three CRC patients (FIG. 23(c)). Primers (LRR1F: TGAGGGGAAAGCCACTGTTC (SEQ ID NO: 43) and LRR1R: TTCAGACAGAATCTTCCACAAACAC (SEQ ID NO: 44)) were designed in the flanking region to identify which LRR1 splice isoform is expressed. These primers bind to sequences that flank the LRR1 alternatively-spliced exon that was identified and the tumour-associated splice variant is 148 bp.

The presence of the LRR1 tumour-associated splice variant was confirmed to be more highly expressed in a number of CRC cell lines (Colo-205, DLD-1, HCT15, HCT116, HT29, RKO and SW480) compared to normal colon tissue (FIG. 23(c)). Using RNA that was isolated from matched adjacent normal and tumour tissue from three different CRC patients, cDNA was prepared and used for RT-PCR to detect LRR1 splice isoforms. Two of these CRC patients show increased expression of the LRR1 tumour-associated splice isoform (FIG. 23(c)). Again, as was described in Example 5, this RT-PCR can be used for detection or diagnostic purposes.

### Example 18

Validation of ZNF670 Splice Variant Antigen in CRC

A ZNF670 splice variant peptide that binds to HLA-A11 (SEQ ID NO: 37) was identified as described in Examples 12 and 13. Aberrant splicing of ZNF670 was observed in 8 out of 37 CRC patients and median change in PSI was 0.362 between tumour and normal samples in the discovery cohort (FIG. 20(a)).

The PSI values for individual normal and tumour samples from CRC patients are shown in FIG. 24(a). Only samples which have sufficient junction counts are shown in this figure. FIG. 24(b) shows the sashimi plot for normal and tumour outlier samples (as was also described in Example 1), each sashimi plot shows the average read density of these samples. The sashimi plot for tumour samples show that there is increased skipping of an exon. The tumour-associated splice event for ZNF670 that was identified consists of skipping of an exon (chr1:247130997-247131094:−, SEQ ID NO: 45).

In order to experimentally verify the presence of the ZNF670 splice isoform in CRC, RT-PCR was performed on cell lines and in tissue samples (matched tumour and adjacent normal samples) from three CRC patients (FIG. 24(c)). Primers (ZNF670F: TTCATTCCAAAAAGTGATGCTGAG (SEQ ID NO: 46) and ZNF670R: CAACATGGAAGAACAATCTTCCTITC (SEQ ID NO: 47)) were designed in the flanking region to identify which ZNF670 splice isoform is expressed. These primers bind to sequences that flank the ZNF670 alternatively-spliced exon that was identified and the tumour-associated splice variant is 283 bp.

The presence of the ZNF670 tumour-associated splice variant was confirmed to be more highly expressed in a number of CRC cell lines (DLD-1, HCT15, and HCT116) compared to normal colon tissue (FIG. 24(c)). Using RNA that was isolated from matched adjacent normal and tumour tissue from three different CRC patients, cDNA was prepared and used for RT-PCR to detect ZNF670 splice isoforms. Two of these CRC patients show increased expression of the ZNF670 tumour-associated splice isoform (FIG. 24(c)). As was described in Example 5, this RT-PCR can be used for detection or diagnostic purposes.

The ZNF670 tumour-associated splice variant that was identified involves the skipping of an exon with 98 nucleotides (SEQ ID NO: 45). Alternative splicing of this exon has not been observed before based on current gene annotation (Gencode version 34 or RefSeq) and represents a novel splice isoform (FIG. 24(d)). As shown in FIG. 24(d), skipping of this exon causes a change in reading frame for the downstream exon, which leads to formation of the HLA-A11 binding peptide that was identified in FIG. 20(a).

### Example 19

Validation of GRINA Splice Variant Antigen in CRC

A GRINA splice variant peptide that binds to HLA-A11 (SEQ ID NO: 38) was identified as described in Examples 12 and 13. Aberrant splicing of GRINA was observed in 10 out of 37 CRC patients and median change in PSI was 0.248 between tumour and normal samples in the discovery cohort (FIG. 20(a)).

The PSI values for individual normal and tumour samples from CRC patients are shown in FIG. 25(a). Only samples that have sufficient junction counts are shown in this figure. FIG. 25(b) shows the sashimi plot for normal and tumour outlier samples (as was described in Example 1); each sashimi plot shows the average read density of these samples. The sashimi plot for tumour samples shows that there is increased excision of an intron. The tumour-associated splice event for GRINA that was identified consists of excision of an intron (chr8:145,065,973-145,066,412:+, SEQ ID NO: 48).

In order to experimentally verify the presence of the GRINA splice isoform in CRC, RT-PCR was performed on cell lines and in tissue samples (matched tumour and adjacent normal samples) from three CRC patients (FIG. 25(c)). Primers (GRINAF: GGTCCCCCATCCTACTATGACAAC (SEQ ID NO: 49) and GRINAR: GAATGGCGAAGATGAAGAGCAC (SEQ ID NO: 50)) were designed in the flanking region to identify which GRINA splice isoform is expressed. These primers bind to sequences that flank the GRINA aberrant splicing event that was identified, and the tumour-associated splice variant is 286 bp.

The presence of the GRINA tumour-associated splice variant was confirmed to be expressed in one CRC cell lines (HCT116) compared to normal colon tissue (FIG. 25(c)). Using RNA that was isolated from matched adjacent normal and tumour tissue from three different CRC patients, cDNA was prepared and used for RT-PCR to detect GRINA splice isoforms. Two of these CRC patients show increased expression of the GRINA tumour-associated splice isoform (FIG. 25(c)). Again, as was described in Example 5, this RT-PCR can be used for detection or diagnostic purposes.

### Example 20

Immunogenicity of Identified CRC HLA-A11 SVPs

Antigen specific T-cells for LRR1, GRINA, and ZNF670 were initially identified in the SVP/HLA-A11 tetramer CyTOF screen (as described in Example 13). The immunogenicity of these targets was further assessed by testing whether antigen specific T-cells could be expanded in the PBMC of healthy donors who were HLA-A11 positive. PBMC were obtained from healthy donors and an aliquot was used to isolate monocytes (CD14 positive selection kit, STEMCELL Technologies) for subsequent differentiation to dendritic cells. Briefly, differentiation of monocytes to dendritic cells was carried out by culturing the isolated CD14 cells with IL4 (10 ng/ml) and GM-CSF (800 IU/ml) for 3 days and maturating the dendritic cells with IL4 (10 ng/ml), GM-CSF (800 IU/ml), LPS (10 ng/ml), IFN-γ (100 IU/ml), and the LRR1, GRINA and ZNF670 HLA-A11 SVP (2.5 μM) overnight. These monocyte derived dendritic cells were then cultured with CD8+ T cells which were isolated from another aliquot of PBMCs from the same donor using EasySep CD8 T cell isolation kit, STEMCELL Technologies. After 10 days of co-culture, expansion of antigen-specific T cells was detected by staining with tetramers (labelled with PE and APC) that have been loaded with the LRR1, GRINA and ZNF670 HLA-A11 SVP. FIG. 26 shows the results of FACS analysis for antigen-specific T-cells for LRR1, GRINA and ZNF670; these antigen-specific T cells would be expected to be double positive for PE and APC. Antigen-specific T-cells for these SVPs are not observed in CD8+ T cells in unstimulated PBMCs from healthy donors, whereas SVP-specific T-cells can be observed when they have been co-cultured with monocyte-derived dendritic cells that have been loaded with the SVP. In summary, antigen-specific CD8 T cells for LRR1, GRINA and ZNF670 were able to be generated in healthy donors, showing that these SVPs are immunogenic.

### Example 21

Prediction of HLA-A24 Binding Peptides from Shared Alternative Spliced Variants and Identification of Splice Variant-Specific CD8+ T Cells in Colorectal Carcinoma Patients

Peptides derived from shared alternative splice variants that could bind to HLA-A24 were identified as was described in Example 12. From this analysis, 75 SVPs that could bind to HLA-A24 were identified. These SVPs were derived from 55 splice events that are shared amongst patient sub-groups.

To determine whether CRC patients have CD8+ T cells that target any of the 75 SVPs that bind to HLA-A24, a CyTOF screen was conducted on a new cohort of 10 HLA-A24 positive CRC patients (validation cohort) using MHC tetramer staining of PBMC (procedure for performing this screen is similar to that described in Example 13). In total, antigen-specific CD8+ T cells that target eight splice variant peptides were identified from this screen. FIG. 27(a) shows a summary of the peptide sequences, frequency of the antigen specific CD8+ T cells, median change in PSI, occurrence and coordinates of the exons for two of these SVA targets.

These SVAs were derived from aberrant splicing of LRR1 and MZF1 with the following peptide sequences: SYHSIPSLPRF (SEQ ID NO: 51) and KWPPATETL (SEQ ID NO: 52). Both of these SVAs were detected in two different cohorts of CRC patients (discovery and validation cohort).

The coordinates of the exons or introns that are aberrantly spliced and tumour-associated isoforms are shown in FIG. 27(b). For the splice event that gave rise to the LRR1 SVA, it is an exon skip/inclusion event that results in the skipping of an exon (chr14:50074118-50074839:+(SEQ ID NO: 42)). This results in transcripts that skip the exon (chr14:50069088-50069186:+@chr14:50080974-50081389:+). For the splice event that gave rise to the MZF1 SVA, it is an intron retention event that results in the retention of an intron (chr19:59,081,895-59,082,360:−(SEQ ID NO: 53)). This results in transcripts that contain the intron retention event (chr19:59081711-59082796:−). All of the coordinates described above are based on GRCh37/hg19 genome assembly.

### Example 22

Candidate Antigenic Regions can Produce Peptides that Bind to Different HLA-Alleles

The LRR1 candidate antigenic region is relatively large due to changes in frame caused by changes in splicing (as described in Example 1 and shown in FIG. 5). The LRR1 candidate antigenic region gives rise to two peptides (SEQ ID NO: 36 and SEQ ID NO: 51) that bind to HLA-A11 as well as HLA-A24 (FIG. 23(d)). Antigen-specific T cells for these two SVPs from the same splice event were detected in different CRC patients (FIG. 20 and FIG. 27). This further indicates that this SVA is shared and immunogenic across patients as well as different HLA types.

### Example 23

Validation of MZF1 Splice Variant Antigen in CRC

A MZF1 splice variant peptide that binds to HLA-A24 (SEQ ID NO: 52) was identified as described in Examples 12 and 13. Aberrant splicing of MZF1 was observed in 6 out of 37 CRC patients and median change in PSI was −0.228 between tumour and normal samples in the discovery cohort (FIG. 27(a)).

The PSI values for individual normal and tumour samples from CRC patients are shown in FIG. 28(a). Only samples which have sufficient junction counts are shown in this figure. FIG. 28(b) shows the sashimi plot for normal and tumour outlier samples (as described in Example 1); each sashimi plot shows the average read density of these samples. The sashimi plot for tumour samples shows that there is increased skipping of an exon. The tumour-associated splice event for MZF1 that was identified shows the retention of an intron (chr19: 59081895-59082360:−, SEQ ID NO: 53).

In order to experimentally verify the presence of the MZF1 splice isoform in CRC, RT-PCR was performed on cell lines and in tissue samples (matched tumour and adjacent normal samples) from three CRC patients (FIG. 28(c)). Primers (MZF1F: GCACTGCCCCCTGAGATCCAG (SEQ ID NO: 54) and MZF1R: CTITCACCTGCAGGCCCAGTG (SEQ ID NO: 55)) were designed in the flanking region to identify which MZF1 splice isoform is expressed. These primers bind to sequences that flank the MZF1 alternative splicing event and the tumour-associated splice variant is 737 bp.

The presence of the MZF1 tumour-associated splice variant was confirmed to be more highly expressed in a number of CRC cell lines (HCT15, HCT116, HT29, and SW480) compared to normal colon tissue (FIG. 28(c)). Using RNA that was isolated from matched adjacent normal and tumour tissue from three different CRC patients, cDNA was prepared and used for RT-PCR to detect MZF1 splice isoforms. One of these CRC patients shows increased expression of the MZF1 tumour-associated splice isoform (FIG. 28(c)). As was described in Example 5, this RT-PCR can be used for detection or diagnostic purposes.

### Example 24

Identification of Shared Candidate Antigens and their Cognate T Cells in Head and Neck Squamous Cell Carcinoma

Head and neck squamous cell carcinoma (HNSC) primary tumour samples (31) and matched normal samples (16) were taken from non-malignant tissue adjacent to tumour and these samples constituted the discovery cohort. Deep RNA sequencing (100 million Paired-End reads) and mRNA splicing analysis using rMATs (only sequencing reads that mapped to splice junctions were used for analysis) were performed on these samples to identify tumour-associated alternative splicing events. Selection criteria (at least 20% change in splicing (ΔPSI), occurrence in at least 5 patients and junction counts for inclusion/skipping must be >5) were applied, which yielded a list of 1418 splice events that resulted in protein coding changes.

These tumour-associated splice variants, which are shared in HNSC patient subpopulations or subgroups, were used for the identification of candidate antigenic regions (FIG. 29(a) and FIG. 29(b)). Candidate antigenic regions from these tumour-associated splice variants were then used to identify 8-11 amino acid-long peptides that could bind to common HLA alleles: HLA-A02; HLA-A11 and; HLA-A24 (FIG. 29(c)). NetMHCpan 3 and 4 were used for the identification of these 8-11 amino acid-long peptides (as described in Example 12). Some of these tumour-associated splice events contain one or more peptides that bind to different HLA alleles. This is similarly observed for the LRR1 SVA that was identified in the CRC SVP/Tetramer CyTOF screens (as described in Example 22), highlighting the utility of identifying candidate antigenic regions.

### Example 25

Shared Antigens Identified in Colorectal Carcinoma are Also Present in Head and Neck Squamous Cell Carcinoma

Cross-referencing the tumour-associated splice variants present in HNSC showed that CAMKK1, LRR1 and GRINA SVPs (SEQ ID NO: 35, SEQ ID NO: 36, SEQ ID NO: 51 and SEQ ID NO: 38), initially identified in the CRC SVP/Tetramer CyTOF screen (as described in Example 13 and Example 21), are also found in HNSC. The occurrence and change in PSI for these SVAs in HNSC patients are shown in FIG. 30.

The PSI values for individual normal and tumour samples from HNSC patients and sashimi plots for CAMKK1 in HNSC patients are shown in FIG. 22(f) and FIG. 22(g). Tumour-associated splice variants for CAMKK1 were also able to be detected in cell lines that had been derived from HNSC patients (FIG. 22(h)). The PSI values for individual normal and tumour samples from HNSC patients and sashimi plots for LRR1 in HNSC patients are shown in FIG. 23(e) and FIG. 23(f). Tumour-associated splice variants for LRR1 were also able to be detected in cell lines that had been derived from HNSC patients (FIG. 23(g)). The PSI values for individual normal and tumour samples from HNSC patients and sashimi plots for GRINA in HNSC patients are shown in FIG. 25(d) and FIG. 25(e). Tumour-associated splice variants for GRINA were also able to be detected in cells lines that had been derived from HNSC patients (FIG. 25(f)). All three of these SVAs were detected in two different cancer types.

### Example 26

Characterizing and/or Treating a Splice Variant Antigen (SVA) Positive Cancer in a Patient.

Patients having recurrent/refractory or metastatic cancer may express a splice variant antigen such as MARK3. For example, a sample of cancerous tissue may be tested for the expression of MARK3. This can be done by RT-PCR as described in Example 5. The patient can also be tested for the expression of HLA (e.g. HLA-A11).

Treatment of patients may be carried out by expansion of splice variant antigen-specific T lymphocytes (such as MARK3-specific T lymphocytes) from the patient, as described in Example 6, and administering these expanded T lymphocytes back into the patient.

Prior to administering these T lymphocytes, the patient may be treated with cyclophosphamide and fludarabine.

Patients having recurrent/refractory or metastatic cancer that express other splice variant antigens (such as NBPF9, PARD3, ZC3HAV1, YAF2, CAMKK1, LRR1, ZNF670, GRINA or MZF1) may be similarly characterized and/or treated.

### REFERENCES

Damato B E, Dukes J, Goodall H, Carvajal R D. Tebentafusp: T Cell Redirection for the Treatment of Metastatic Uveal Melanoma. Cancers (Basel). 2019 Jul. 11; 11 (7). pii: E971. doi: 10.3390/cancers11070971. Review. PubMed PMID: 31336704; PubMed Central PMCID: PMC6679206
Walseng E, Wälchli S, Fallang L E, Yang W, Vefferstad A, Areffard A, Olweus J. Soluble T-cell receptors produced in human cells for targeted delivery. PLoS One. 2015 Apr. 13; 10 (4):e0119559. doi: 10.1371/journal.pone.0119559. eCollection 2015. PubMed PMID: 25875651; PubMed Central PMCID: PMC4395278.
Wang G C, Dash P, McCullers J A, Doherty P C, Thomas P G. T cell receptor up diversity inversely correlates with pathogen-specific antibody levels in human cytomegalovirus infection. Sci Transl Med. 2012 Apr. 4; 4(128):128ra42. doi: 10.1126/scitranslmed.3003647. PubMed PMID: 22491952; PubMed Central PMCID: PMC3593633.
Zhang G, Wang L, Cui H, Wang X, Zhang G, Ma J, Han H, He W, Wang W, Zhao Y, Liu C, Sun M, Gao B. Anti-melanoma activity of T cells redirected with a TCR-like chimeric antigen receptor. Sci Rep. 2014 Jan. 6; 4:3571. doi: 10.1038/srep03571. PubMed PMID: 24389689; PubMed Central PMCID: PMC3880964.

1. A method of identifying one or more shared candidate antigens for characterising and/or treating a medical condition, the method including:
obtaining transcriptomic data for test samples from a first cohort of patients having the medical condition;
(ii) obtaining reference transcriptomic data for a set of reference samples;
(iii) determining, by a comparison of the transcriptomic data to the reference transcriptomic data, one or more splice variants that are more highly transcribed in each sample of a subset of the test samples as compared to the reference samples;
(iv) determining, for each said shared splice variant, one or more amino acid sequences that occur in an amino acid translation of the shared splice variant, but not in amino acid translations of corresponding splice variants of the same gene that are transcribed in the reference samples; and
(v) predicting HLA binding of the one or more shared amino acid sequences, or part thereof, to identify the one or more shared amino acid sequences as one or more shared candidate antigens,
wherein the method further includes determining one or more common HLA alleles that occur in more than a predetermined proportion of the first cohort of patients, wherein step (v) includes: generating a plurality of candidate peptides from the one or more amino acid sequences, and predicting binding of the plurality of shared candidate peptides to proteins encoded by the one or more common HLA alleles.

2. A method according to claim 1, wherein step (iv) includes determining non-overlapping nucleotide sequence between the shared splice variant and corresponding splice variants of the same gene, optionally wherein the method further includes determining for each said shared splice variant, prior to step (iv), whether there is a change in reading frame in the first shared splice variant relative to the one or more corresponding splice variants of the same gene.

3. (canceled)

4. A method according to claim 1, wherein the subset comprising the shared splice variant comprises more than a threshold number or more than a threshold percentage of the test samples.

5. (canceled)

6. A method according to claim 1, wherein the set of reference samples includes matched normal samples from the first cohort of patients, and/or wherein the set of reference samples includes samples from a cohort of subjects who do not have the medical condition.

7. (canceled)

8. A method according to claim 1, wherein the medical condition is cancer, optionally wherein the medical condition is gastric cancer, head and neck cancer, colorectal cancer or hepatocellular cancer, further optionally wherein the medical condition is common to a group of patients.

9.-13. (canceled)

14. A method according to claim 1, wherein the method comprises verifying or testing HLA binding of the one or more shared amino acid sequences to identify the one or more shared amino acid sequences as a shared candidate antigen.

15. The method according to claim 1, wherein the method further comprises identifying a shared antigen-T lymphocyte pair, the method comprising:
vi) providing one or more respective labelled biomolecules comprising a label and a peptide comprising the shared candidate antigen;
vii) contacting the one or more labelled biomolecules with one or more samples containing peripheral blood from patients having the medical condition; and
viii) identifying, from the one or more samples, T lymphocytes that are bound to said labelled biomolecules, so as to identify a shared antigen-T lymphocyte pair,
optionally wherein the labelled biomolecules comprise HLA multimers.

16. (canceled)

17. A method according to claim 15, wherein labelled biomolecules containing respective shared candidate antigens are labelled with different respective barcodes, optionally wherein the barcodes are heavy metal barcodes.

18. (canceled)

19. A method according to claim 15, wherein the respective patients having the medical condition are part of a second cohort of patients that does not overlap with the first cohort of patients.

20.-21. (canceled)

22. A method according to claim 1, the method further comprising identifying T lymphocytes that bind specifically to one or more shared candidate antigens, the method comprising:
providing one or more respective labelled biomolecules comprising a label and a respective shared candidate antigen;
(ii) contacting the one or more labelled biomolecules with one or more samples containing peripheral blood from respective patients having the medical condition; and
(iii) identifying, from the one or more samples, T lymphocytes that are bound to said labelled biomolecules.

23. A method according to claim 22, wherein identification of T lymphocytes that are bound to said labelled biomolecules characterises the respective patients as having a medical condition that is associated with the expression of the one or more shared antigens.

24. A method according to claim 22, wherein the method comprises testing the biological function of the T lymphocytes, optionally wherein the method comprises characterising the T lymphocytes to determine whether they are cytotoxic and/or testing whether the shared antigens are immunogenic.

25.-30. (canceled)

31. A method of treating a medical condition in a subject, the method comprising:
(a) isolating a population of T lymphocytes that binds specifically to one or more shared antigens identified according to claim 1 in a subject suffering from the medical condition, and expanding the population of T lymphocytes ex vivo; and
(b) administering the expanded population of T lymphocytes to the subject to treat the medical condition in the subject.

32. An immunomodulatory composition comprising one or more shared antigen identified according to claim 1 and a pharmaceutically acceptable carrier.

33. A composition according to claim 32, wherein the shared antigen is a peptide having at least 80% sequence identity to any one of SEQ ID NOs: 1, 31-38, 51 or 52, or is a nucleic acid encoding a peptide having at least 80% sequence identity to any one of SEQ ID NOs: 1 31-38, 51 or 52.

34. (canceled)

35. A shared antigen-T lymphocyte pair identified according to claim 15, wherein the shared antigen is a MARK3, NBPF9, PARD3, ZC3HAV1, YAF2, CAMKK1, LRR1, ZNG670, GRINA or MZF1 splice variant, the HLA subtype is HLA-A11 or HLA-A24, and the T lymphocyte binds to the shared antigen.

36.-39. (canceled)

40. A T-cell receptor (TCR) that binds to a shared antigen according to claim 1, wherein the shared antigen is bound to a HLA molecule.

41.-50. (canceled)


SEQUENCE LISTING (67 sequences)

SEQ ID NO: 1 - protein, 9 aa, Artificial Sequence
  MARK3 splice variant
RNMSFRFIK

SEQ ID NO: 2 - protein, 10 aa, Artificial Sequence
  EBV peptide sequence
SSCSSCPLSK

SEQ ID NO: 3 - DNA, 21 bp, Artificial Sequence
  MARK3 forward primer
tcccatgaagccacaccattg

SEQ ID NO: 4 - DNA, 21 bp, Artificial Sequence
  MARK3 reverse primer
agcgtagggatcgaggctttg

SEQ ID NO: 5 - DNA, 258 bp, Artificial Sequence
  Exon 23
cactattcctgatcagagaactccagttgcttcaacacacagtatcagtagtgcagccaccccagatcgaatccgcttcccaagaggcactgccagtcgtagcactttccacggccagccccgggaacggcgaaccgcaacatataatggccctcctgcctctcccagcctgtcccatgaagccacaccattgtcccagactcgaagccgaggctccactaatctctttagtaaattaacttcaaaactcacaaggag

SEQ ID NO: 6 - DNA, 948 bp, Artificial Sequence
  Exon 26
tcgcaatgtatctgctgagcaaaaagatgaaaacaaagaagcaaagcctcgatccctacgcttcacctggagcatgaaaaccactagttcaatggatcccggggacatgatgcgggaaatccgcaaagtgttggacgccaataactgcgactatgagcagagggagcgcttcttgctcttctgcgtccacggagatgggcacgcggagaacctcgtgcagtgggaaatggaagtgtgcaagctgccaagactgtctctgaacggggtccggtttaagcggatatcggggacatccatagccttcaaaaatattgcttccaaaattgccaatgagctaaagctgtaacccagtgattatgatgtaaattaagtagcaattaaagtgttttcctgaacactgatggaaatgtatagaataatatttaggcaataacgtctgcatcttctaaatcatgaaattaaagtctgaggacgagagcacgcctgggagcgaaagctggccttttttctacgaatgcactacattaaagatgtgcaacctatgcgccccctgccctacttccgttaccctgagagtcggtgtgtggccccatctccatgtgcctcccgtctgggtgggtgtgagagtggacggtatgtgtgtgaagtggtgtatatggaagcatctccctacactggcagccagtcattactagtacctctgcgggagatcatccggtgctaaaacattacagttgccaaggaggaaaatactgaatgactgctaagaattaaccttaagaccagttcatagttaatacaggtttacagttcatgcctgtggttttgtgtttgttgttttgtgtttttttagtgcaaaaggtttaaatttatagttgtgaacattgcttgtgtgtgtttttctaagtagattcacaagataattaaaaattcactttttctcagtaa

SEQ ID NO: 7 - DNA, 27 bp, Artificial Sequence
  Exon 24
aaacatgtcattcaggtttatcaaaag

SEQ ID NO: 8 - DNA, 45 bp, Artificial Sequence
  Exon 25
gcttccaactgaatatgagaggaacgggagatatgagggctcaag

SEQ ID NO: 9 - DNA, 19 bp, Artificial Sequence
  TRAC primer
gaccagcttgacatcacag

SEQ ID NO: 10 - DNA, 24 bp, Artificial Sequence
  TRAC primer
ctcaggcagtatctggagtcattg

SEQ ID NO: 11 - DNA, 21 bp, Artificial Sequence
  TRAC PCR1
tgctgttgttgaaggcgtttg

SEQ ID NO: 12 - DNA, 21 bp, Artificial Sequence
  TRAC PCR2
tgttgctcttgaagtccatag

SEQ ID NO: 13 - DNA, 19 bp, Artificial Sequence
  TRBC PCR1
cccactgtgcacctccttc

SEQ ID NO: 14 - DNA, 20 bp, Artificial Sequence
  TRBC PCR2
ttctgatggctcaaacacag

SEQ ID NO: 15 - protein, 109 aa, Artificial Sequence
  TRAV6*01 AA sequence
MESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYL

SEQ ID NO: 16 - protein, 11 aa, Artificial Sequence
  TRAJ9*01 AA sequence
GAGTRLFVKAN

SEQ ID NO: 17 - protein, 6 aa, Artificial Sequence
  CDR1 AA sequence
NYSPAY

SEQ ID NO: 18 - protein, 7 aa, Artificial Sequence
  CDR2 AA sequence
IRENEKE

SEQ ID NO: 19 - DNA, 36 bp, Artificial Sequence
  CDR3 nucleotide sequence
tgtgctccgtatactggaggcttcaaaactatcttt

SEQ ID NO: 20 - protein, 12 aa, Artificial Sequence
  CDR3 AA sequence
CAPYTGGFKTIF

SEQ ID NO: 21 - protein, 132 aa, Artificial Sequence
  MARK3 TRA variable region AA sequence
MESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCAPYTGGFKTIFGAGTRLFVKAN

SEQ ID NO: 22 - protein, 272 aa, Artificial Sequence
  MARK3 TRA AA sequence
MESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCAPYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 23 - protein, 110 aa, Artificial Sequence
  TRBV7-9*01 AA sequence
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYL

SEQ ID NO: 24 - protein, 9 aa, Artificial Sequence
  TRAJ1-2*01 AA sequence
GSGTRLTVV

SEQ ID NO: 25 - protein, 5 aa, Artificial Sequence
  CDR1 AA sequence
SEHNR

SEQ ID NO: 26 - protein, 5 aa, Artificial Sequence
  CDR2 AA sequence
FQNEA

SEQ ID NO: 27 - DNA, 42 bp, Artificial Sequence
  CDR3 nucleotide sequence
tgtgccagcagctccccccgggttggctatggctacaccttc

SEQ ID NO: 28 - protein, 14 aa, Artificial Sequence
  CDR3 AA sequence
CASSSPRVGYGYTF

SEQ ID NO: 29 - protein, 133 aa, Artificial Sequence
  MARK3 TRB variable region AA sequence
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSPRVGYGYTFGSGTRLTVV

SEQ ID NO: 30 - protein, 312 aa, Artificial Sequence
  MARK3 TRB AA sequence
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSPRVGYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDSRG

SEQ ID NO: 31 - protein, 9 aa, Artificial Sequence
  NBPF9 splice variant antigen
SSFYALEEK

SEQ ID NO: 32 - protein, 10 aa, Artificial Sequence
  PARD3 splice variant antigen
SQLDFVKTRK

SEQ ID NO: 33 - protein, 9 aa, Artificial Sequence
  ZC3HAV splice variant antigen
LTMAVKAEK

SEQ ID NO: 34 - protein, 9 aa, Artificial Sequence
  YAF2 splice variant antigen
VIVSASRTK

SEQ ID NO: 35 - protein, 9 aa, Artificial Sequence
  CAMKK1 splice variant antigen
VTSPSRRSK

SEQ ID NO: 36 - protein, 9 aa, Artificial Sequence
  LRR1 splice variant antigen
SLPRFGYRK

SEQ ID NO: 37 - protein, 10 aa, Artificial Sequence
  ZNF670 splice variant antigen
SCVSPSSELK

SEQ ID NO: 38 - protein, 9 aa, Artificial Sequence
  GRINA splice variant antigen
SIRQAFIRK

SEQ ID NO: 39 - DNA, 22 bp, Artificial Sequence
  CAMKK1 skipped exon
tgtttgacctcctgagaaaggg

SEQ ID NO: 40 - DNA, 22 bp, Artificial Sequence
  CAMKK1F
gaagctggaccacgtgaatgtg

SEQ ID NO: 41 - DNA, 22 bp, Artificial Sequence
  CAMKK1R
agtactcgaggcccaggatgac

SEQ ID NO: 42 - DNA, 722 bp, Artificial Sequence
  LRR1 skipped exon
gccatttccagcagtttaaaaggtttcctttcagctatgagactggctcatagaggctgtaatgttgatacaccagtttcaacgctcacaccagtgaagacttcagaatttgaaaactttaaaactaaaatggttatcacatccaaaaaagactatcctctaagtaagaattttccatattccttggaacatcttcagacttcttactgtgggcttgtccgagttgatatgcgtatgctttgcttaaaaagccttaggaaattagacttgagtcacaaccatataaaaaagcttccagctacaattggagacctcatacaccttcaagaacttaacctgaatgacaatcacttggagtcatttagtgtagccttgtgtcattctacactccagaagtcacttcggagtttggacctcagcaagaacaaaatcaaggcactccctgtgcagttttgccagctccaggaacttaagaatttaaaacttgacgataatgaattgattcaatttccttgcaagataggacaactaataaaccttcgctttttgtcagcagctcgaaataagcttccatttttgcctagtgaatttagaaatttatcccttgaatacttggatctttttggaaatacttttgaacaaccaaaagtccttccagtaataaagctgcaagcaccattaactttattggaatcttctgcacgaaccatattacataatag

SEQ ID NO: 43 - DNA, 20 bp, Artificial Sequence
  LRR1F
tgaggggaaagccactgttc

SEQ ID NO: 44 - DNA, 25 bp, Artificial Sequence
  LRR1R
ttcagacagaatcttccacaaacac

SEQ ID NO: 45 - DNA, 98 bp, Artificial Sequence
  ZNF670 skipped exon
ccatgtgaagatgtgcttgcttcccctttgccttctgccatgattctaagtttcctgaggcctccccagaagcagaagcatgtaaagcccacagaacc

SEQ ID NO: 46 - DNA, 24 bp, Artificial Sequence
  ZNF670F
ttcattccaaaaagtgatgctgag

SEQ ID NO: 47 - DNA, 26 bp, Artificial Sequence
  ZNF670R
caacatggaagaacaatcttcctttc

SEQ ID NO: 48 - DNA, 440 bp, Artificial Sequence
  GRINA1 skipped exon
gtgggtaggggcatctccaaggcggtgggggctgtggctcccagcggatgactctgagcggctccttccccaggtgttcctagtgctgaccttgcagctgtcggtgaccctgtccacggtgtctgtgttcacttttgttgcggaggtgaagggctttgtccgggagaatgtctggacctactatgtctcctatgctgtcttcttcatctctctcatcgtcctcagctgttgtggggacttccggcgaaagcacccctggaaccttgttgcactggtaacccccaaacctgagcctctgtgcctgggtccggccatgcagtcccacacgcccactcttccgggcctggtcaccgtggttctccttgtgctcatgaggcaggggccggcaggggtggcatgggggcacacacccaaggtcagcctgtgtctcccaactgcag

SEQ ID NO: 49 - DNA, 24 bp, Artificial Sequence
  GRINAF
ggtcccccatcctactatgacaac

SEQ ID NO: 50 - DNA, 22 bp, Artificial Sequence
  GRINAR
gaatggcgaagatgaagagcac

SEQ ID NO: 51 - protein, 11 aa, Artificial Sequence
  LRR1 splice variant antigen
SYHSIPSLPRF

SEQ ID NO: 52 - protein, 9 aa, Artificial Sequence
  MZF1 spliced variant antigen
KWPPATETL

SEQ ID NO: 53 - DNA, 466 bp, Artificial Sequence
  MZF1 splice variant antigen
gtgagtgagtgtccacgagtggggttcaggactggagcatcatccagctcggcctcaccctgggcagcaccacgctttgccagacacacgttctcccttgtggtacagaagtggccacctgcaacagagaccctgtgcaaggctgcccagggcagagggattccagggccagactccccagcccattcctgcatcacctgcagtcacgtggacataggagctgcaggctaggggaatatggggaaggtactggaaggccacgatgtcagagcaggggagggactgcaggtggtcccttcattctttgctcagggcctaggggaggtaggtccttggaggacctagtcagtaggtatttacaaggcaggcccctgggatcctacagtgggatgggcacccacaaaccccaggttgcggccagccttactctctggtaggacttctgatggtgggggcacctccccag

SEQ ID NO: 54 - DNA, 21 bp, Artificial Sequence
  MZF1F
gcactgccccctgagatccag

SEQ ID NO: 55 - DNA, 21 bp, Artificial Sequence
  MZF1R
ctttcacctgcaggcccagtg

SEQ ID NO: 56 - DNA, 348 bp, Artificial Sequence
  Normal associated DNA sequence of CAMKK1
gtcctggatgacccagctgaggacaacctctatttggtgtttgacctcctgagaaaggggcccgtcatggaagtgccctgtgacaagcccttctcggaggagcaagctcgcctctacctgcgggacgtcatcctgggcctcgagtacttgcactgccagaagatcgtccacagggacatcaagccatccaacctgctcctgggggatgatgggcacgtgaagatcgccgactttggcgtcagcaaccagtttgaggggaacgacgctcagctgtccagcacggcgggaaccccagcattcatggcccccgaggccatttctgattccggccagagcttcagtgggaag

SEQ ID NO: 57 - protein, 116 aa, Artificial Sequence
  Normal protein sequence of CAMKK1
VLDDPAEDNLYLVFDLLRKGPVMEVPCDKPFSEEQARLYLRDVILGLEYLHCQKIVHRDIKPSNLLLGDDGHVKIADFGVSNQFEGNDAQLSSTAGTPAFMAPEAISDSGQSFSGK

SEQ ID NO: 58 - DNA, 326 bp, Artificial Sequence
  Tumour associated DNA sequence of CAMKK1
gtcctggatgacccagctgaggacaacctctatttgggcccgtcatggaagtgccctgtgacaagcccttctcggaggagcaagctcgcctctacctgcgggacgtcatcctgggcctcgagtacttgcactgccagaagatcgtccacagggacatcaagccatccaacctgctcctgggggatgatgggcacgtgaagatcgccgactttggcgtcagcaaccagtttgaggggaacgacgctcagctgtccagcacggcgggaaccccagcattcatggcccccgaggccatttctgattccggccagagcttcagtgggaag

SEQ ID NO: 59 - protein, 65 aa, Artificial Sequence
  Tumour associated protein sequence of CAMKK1
VLDDPAEDNLYLGPSWKCPVTSPSRRSKLASTCGTSSWASSTCTARRSSTGTSSHPTCSWGMMGT

SEQ ID NO: 60 - DNA, 993 bp, Artificial Sequence
  Normal associated DNA sequence of LRR1
gagcctcctgtggatatctgtctaagtaaggccatttccagcagtttaaaaggtttcctttcagctatgagactggctcatagaggctgtaatgttgatacaccagtttcaacgctcacaccagtgaagacttcagaatttgaaaactttaaaactaaaatggttatcacatccaaaaaagactatcctctaagtaagaattttccatattccttggaacatcttcagacttcttactgtgggcttgtccgagttgatatgcgtatgctttgcttaaaaagccttaggaaattagacttgagtcacaaccatataaaaaagcttccagctacaattggagacctcatacaccttcaagaacttaacctgaatgacaatcacttggagtcatttagtgtagccttgtgtcattctacactccagaagtcacttcggagtttggacctcagcaagaacaaaatcaaggcactccctgtgcagttttgccagctccaggaacttaagaatttaaaacttgacgataatgaattgattcaatttccttgcaagataggacaactaataaaccttcgctttttgtcagcagctcgaaataagcttccatttttgcctagtgaatttagaaatttatcccttgaatacttggatctttttggaaatacttttgaacaaccaaaagtccttccagtaataaagctgcaagcaccattaactttattggaatcttctgcacgaaccatattacataataggattccatatggctctcatatcattccattccatctctgccaagatttggataccgcaaaaatttgtgtttgtggaagattctgtctgaactctttcattcaaggaactactaccatgaatctgcattctgttgcccacactgtggtcttagtagataatttgggtggtactgaagcacctattatctcttatttctgttctctaggctgttatgttaattcctctgatatgttaaagtaa

SEQ ID NO: 61 - protein, 330 aa, Artificial Sequence
  Normal protein sequence of LRR1
EPPVDICLSKAISSSLKGFLSAMRLAHRGCNVDTPVSTLTPVKTSEFENFKTKMVITSKKDYPLSKNFPYSLEHLQTSYCGLVRVDMRMLCLKSLRKLDLSHNHIKKLPATIGDLIHLQELNLNDNHLESFSVALCHSTLQKSLRSLDLSKNKIKALPVQFCQLQELKNLKLDDNELIQFPCKIGQLINLRFLSAARNKLPFLPSEFRNLSLEYLDLFGNTFEQPKVLPVIKLQAPLTLLESSARTILHNRIPYGSHIIPFHLCQDLDTAKICVCGRFCLNSFIQGTTTMNLHSVAHTVVLVDNLGGTEAPIISYFCSLGCYVNSSDMLK

SEQ ID NO: 62 - DNA, 271 bp, Artificial Sequence
  Tumour associated DNA sequence of LRR1
gagcctcctgtggatatctgtctaagtaaggattccatatggctctcatatcattccattccatctctgccaagatttggataccgcaaaaatttgtgtttgtggaagattctgtctgaactctttcattcaaggaactactaccatgaatctgcattctgttgcccacactgtggtcttagtagataatttgggtggtactgaagcacctattatctcttatttctgttctctaggctgttatgttaattcctctgatatgttaaagtaa

SEQ ID NO: 63 - protein, 62 aa, Artificial Sequence
  Tumour associated protein sequence of LRR1
EPPVDICLSKDSIWLSYHSIPSLPRFGYRKNLCLWKILSELFHSRNYYHESAFCCPHCGLSR

SEQ ID NO: 64 - DNA, 515 bp, Artificial Sequence
  Normal associated DNA sequence of ZNF670
ttttgtcttcttatcttactgaagacattttgccagagcagggcctgcaagtttcattccaaaaagtgatgctgagaagatatgaaagatgttgtcttgagaaattacgcttaaggaatgactgggaaattgtggccatgtgaagatgtgcttgcttcccctttgccttctgccatgattctaagtttcctgaggcctccccagaagcagaagcatgtaaagcccacagaaccggtccagtcgtcgaccatcgagctgctttgaagaccctgcggaggttccgtcgctgcttgaacacagtgcctggactccctgctccgtgaggcgaatttacaccgggtccagctgcgtcagcccgagttctgaattaaaacatgcctcccacagaatctatttctgatgaatatgaaaggaagattgttcttccatgttggtttttattgagatatctctttttttcccttttcatttggttgtcctaactccatgaatagaaaagaaaatgtattaattca

SEQ ID NO: 65 - protein, 46 aa, Artificial Sequence
  Normal protein sequence of ZNF670
LSSYLTEDILPEQGLQVSFQKVMLRRYERCCLEKLRLRNDWEIVAM

SEQ ID NO: 66 - DNA, 417 bp, Artificial Sequence
  Tumour associated DNA sequence of ZNF670
ttttgtcttcttatcttactgaagacattttgccagagcagggcctgcaagtttcattccaaaaagtgatgctgagaagatatgaaagatgttgtcttgagaaattacgcttaaggaatgactgggaaattgtggggtccagtcgtcgaccatcgagctgctttgaagaccctgcggaggttccgtcgctgcttgaacacagtgcctggactccctgctccgtgaggcgaatttacaccgggtccagctgcgtcagcccgagttctgaattaaaacatgcctcccacagaatctatttctgatgaatatgaaaggaagattgttcttccatgttggtttttattgagatatctctttttttcccttttcatttggttgtcctaactccatgaatagaaaagaaaatgtattaattca

SEQ ID NO: 67 - protein, 99 aa, Artificial Sequence
  Tumour associated protein sequence of ZNF670
LSSYLTEDILPEQGLQVSFQKVMLRRYERCCLEKLRLRNDWEIVGSSRRPSSCFEDPAEVPSLLEHSAWTPCSVRRIYTGSSCVSPSSELKHASHRIYF